(19) United States
(12) Patent Application Publication
(10) Pub. No.: US 2008/0145313 A1
(43) Pub. Date: Jun. 19, 2008

(54) Compositions and Methods for the Treatment and Prevention of Neoplastic Disorders

(75) Inventors: James D. Watson, Auckland (NZ); Elizabeth S. Visser, Auckland (NZ); Jun Hiyama, Auckland (NZ); llkka J. Havukkala, Auckland (NZ); Glen Reid, Auckland (NZ); Annette Lasham, Auckland (NZ)

Correspondence Address: SPECKMAN LAW GROUP PLLC, 1201 THIRD AVENUE, SUITE 330, SEATTLE, WA, 98101, US

(73) Assignee: Genesis Research & Development Corporation Limited, Auckland (NZ)

(21) Appl. No.: 11/847,733

(22) Filed: Aug. 30, 2007

Related U.S. Application Data
(60) Provisional application No. 60/824,047, filed on Aug. 30, 2006.
(60) Provisional application No. 60/841,359, filed on Aug. 30, 2006.
(60) Provisional application No. 60/842,336, filed on Aug. 30, 2006.
(60) Provisional application No. 60/841,733, filed on Aug. 31, 2006.
(60) Provisional application No. 60/910,160, filed on Apr. 4, 2007.
(60) Provisional application No. 60/910,424, filed on Apr. 5, 2007.
(60) Provisional application No. 60/910,436, filed on Apr. 5, 2007.

(51) Int. Cl. A61K 49/00; C07H 21/00; C07H 21/02; A61P 35/00; A61K 31/7052; G01N 33/48

(52) U.S. Cl. 424/9.2; 536/22.1; 536/23.1; 514/44; 436/94

(57) ABSTRACT

Compositions for the treatment and/or prevention of cancer in a mammal by means of RNA interference are provided, together with methods for the use of such compositions. The compositions comprise a small interfering nucleic acid molecule (siNA) that suppresses expression of a target gene that: (a) is involved in an energy metabolism pathway, such as the glycolysis pathway, within a tumor cell; (b) is involved in the biosynthesis of nucleotides, in glycogen metabolism or is an electron carrier that works with ATP to provide metabolic energy; or (c) is involved in transport of sugars, amino acids, and/or water-soluble vitamins, control of intercellular pH, or drug transport within a tumor cell.

### REFERENCE TO RELATED APPLICATIONS

This application claims priority under 35 U.S.C. §119(e) to U.S. Provisional Patent Applications No. 60/824,047, filed Aug. 30, 2006; No. 60/841,359, filed Aug. 30, 2006; No. 60/842,336, filed Aug. 30, 2006; No. 60/841,733, filed Aug. 31, 2006; No. 60/910,160 filed Apr. 4, 2007; No. 60/910,424 filed Apr. 5, 2007; and No. 60/910,436 filed Apr. 5, 2007.

### FIELD OF THE INVENTION

The present invention relates to the treatment of disorders such as cancer by means of RNA interference (RNAi). More specifically, the present invention relates to the targeted delivery of small nucleic acid molecules that are capable of mediating RNAi against genes that are active in glucose metabolism pathways, such as the glycolytic pathway, energy metabolism pathways, and/or in transport pathways, such as glucose transporters, equilibrative nucleoside transporters, amino acid transporters, vitamin transporters, and transporters controlling intracellular pH, as well as ion and drug pumps.

### BACKGROUND OF THE INVENTION

Cancer is a group of diseases characterized by the uncontrolled growth and spread of abnormal cells. Cancer cells evolve from normal cells and acquire traits that enable sustained proliferation, infinite replication, enhanced mobility, and invasiveness, as well as insensitivity to growth inhibition and programmed cell death cues. Such traits are often linked to the over-expression of genes that encode growth and survival promoting proteins.

Cancer continues to be one of the leading causes of death in the United States and throughout the world. More than half a million people die in the US every year from cancer. Current treatments for cancer include chemotherapy, radiation treatment and/or surgical removal of tumors. There are significant limitations to all these techniques. For example, once a tumor has metastasized, it is almost impossible to surgically remove all cancerous cells. Radiation therapy can damage normal tissue surrounding a tumor, and also may not be sufficient to kill all the cancer cells. Similarly, chemotherapy often causes damage to non-cancerous, as well as cancerous, cells, leading to negative side effects. In addition, treatment of cancer with chemotherapeutic drugs often results in the emergence of drug-resistant cancer cells that no longer respond to chemotherapy. When the drug target is a protein, such as thymidylate synthase, resistance can result from increased expression of the target protein or from a mutation in the protein so that it is no longer a functional drug target. There thus remains a need for improved compositions and methods for the treatment of cancer.

### Energy Metabolism in Cancer Cells

Normal cells employ two main pathways to generate energy in the form of adenosine triphosphate (ATP), namely oxidative phosphorylation in mitochondria and glycolysis in the cytoplasm. All normal mammalian cells use both these pathways but rely overwhelmingly on oxidative phosphorylation, switching to glycolysis only at times of oxygen deprivation (hypoxia). Glycolysis is the sequence of reactions that converts glucose into pyruvate. In aerobic conditions, pyruvate enters the citric acid cycle (Kreb's cycle) in the mitochondria where it yields acetyl coenzyme A which, in turn, is completely oxidized to CO₂ and H₂O. Under anaerobic conditions, pyruvate is converted to lactate in the cytoplasm. FIG. 1 shows the linear pathway of enzymes involved in glycolysis together with the sites of ATP utilization and generation.

In most normal mammalian cells, lactate production is suppressed and glycolysis is slowed by environmental oxygen, referred to as the “Pasteur effect”. In 1931 Warburg showed that, in contrast to normal cells, many tumors rely on glycolysis in the presence of oxygen, thereby producing lactate. This is known as the “Warburg effect”, or aerobic glycolysis. It is believed that highly malignant tumors, such as those derived from liver, kidney and brain, may obtain over 50% of their energy via glycolysis. Today, as in the 1930's, biologists question whether a shift in energy production from oxidative phosphorylation to glycolysis is a fundamental property of cancer cells, or a by-product of a normal cell's transformation into a cancer cell.

In short-term or mild hypoxia, normal cell viability is maintained by changes in the expression of genes for erythropoietin (EPO), the angiogenic vascular endothelial growth factor (VEGF), transferrin receptors and other proteins which collectively allow for the development of a more effective oxygen (and nutrient) supply, thereby allowing the cells to adjust to conditions of low oxygen. Another group of genes involved in the response to hypoxia, namely glycolytic enzymes and glucose transporters, controls glycolysis. However, prolonged and/or severe hypoxia generally leads to cell death due to the activation of apoptotic pathways including the wild-type p53 gene. If the effect of the transcription factor p53 is abolished due to a mutation, a cell becomes resistant to hypoxia-induced apoptosis. Many tumor cells are resistant to triggers that generally lead to cell death, including severe hypoxia.

In tumors, enhanced cell proliferation and a growing cell mass limits the supply of oxygen by existing blood vessels, leading to hypoxia. Hypoxia became a major field of cancer research when the first hypoxia-inducible transcription factor (HIF-1) was identified in 1991. HIF-1 regulates many genes, including all of the glycolytic enzymes, and is overexpressed in many different types of tumors. HIF-1 can be induced not only by hypoxia, but also by defects in the Akt-1 pathway, even under aerobic conditions, and by various oncogenes. The transcription factor HIF-1 is a heterodimer of two subunits, referred to as HIF-1α and HIF-1β. This heterodimer binds to a short conserved DNA sequence found in the promoters of a number of genes. For example, HIF-1 binds a short conserved DNA sequence at the end of the hexokinase II (HK-II) promoter, thereby activating HK-II expression. HIF-1 belongs to the basic helix-loop-helix (bHLH) superfamily of transcription factors, and also contains an additional auxiliary dimerization site known as the PAS domain. The HLH domain and amino terminus of the PAS domain are required for dimerization, with the basic domain and the carboxy-terminus of PAS being required for DNA binding (Schmid et al., J. Cell. Mol. Med. 8:423-431, 2004).

HIF-1β (also known as ARNT) is a nuclear protein that is constitutively expressed independently of O₂ tension. In contrast, HIF-1α is a cytoplasmic protein responsive to O₂ levels. A number of splice variants of HIF-1α (have been identified (Mazure et al., Biochem. Pharmacol. 68:971-980, 2004). In well-oxygenated cells, HIF-1α is continuously degraded by the ubiquitin-proteasome system. This degradation process takes place when certain conserved prolyl residues of HIF-1α are hydroxylated, a modification requiring O₂-dependent enzyme activity. Only HIF-1α containing these modified prolyl sites binds to the von Hippel-Lindau protein, which is the recognition component of an E3 ubiquitin ligase that finally targets HIF-1α for proteasomal degradation. Under hypoxic conditions, the hydroxylation and subsequent degradation of HIF-1α is inhibited and HIF-1α subunits translocate to the nucleus where they heterodimerize with HIF-1β subunits. The resultant product is an active HIF-1 protein that binds to specific hypoxic response elements present in target gene promoters, ultimately activating transcription of these genes (see, for example Semenza et al., J. Biol. Chem., 271:32529-32537, 1996). US patent publication no. 20050074430 describes a recombinant virus engineered to contain a hypoxia/HIF-responsive element (HRE), specifically an HRE from EPO or VEGF.

The protein products of the mutated p53 gene also activate the HIF-1 promoter. In addition, glucose binds an e-box (a glucose-responsive DNA sequence) on the distal end of the HIF-1 promoter, enhancing HK-II production 2-3 fold in the absence of HIF-1.

In the first step in the glycolytic pathway, glucose enters the cell through specific transport proteins and is phosphorylated by ATP to form glucose-6-phosphate (G6P) in a reaction catalyzed by hexokinases. Four hexokinase isoenzymes have been isolated (known as I, II, III and IV). The expression of these isoenzymes is different between normal and cancer cells, with cancer cells generally having high concentrations of HK-II. HK-II has a much higher affinity for glucose than either glucokinase or HK-IV, the enzyme that phosphorylates glucose in normal cells. The effect of HK-II is modulated by G6P via negative feedback inhibition. However, in hypoxic conditions HK-II binds to the outer membrane of mitochondria via the VDAC receptor as shown in FIG. 2, blocking the feedback activity of G6P, and thus maintaining an increased glycolytic rate. It has been shown that 2-deoxy-D-glucose binds and depletes HK-II, and that 3-bromopyruvate strongly inhibits HK-II, both resulting in cancer cell apoptosis.

The wild type p53 protein binds to a specific motif on the HK-II gene promoter for transactivation, with a resulting suppression of cellular transformation. Mutant p53 RNA has been shown to form oligomeric complexes with wild-type p53 protein prior to DNA binding, resulting in loss of affinity of wild-type p53 protein for DNA. In addition to this inactivation effect by the mutant p53 RNA, it has been shown that the mutant p53 can promote growth of parental tumor cells (Mathupala et al., J. Biol. Chem. 272:22776-22780, 1997). Katabi et al. have reported that the HK-II promoter is tumor-specific and may be used for gene-targeted therapy because it is differentially expressed and regulated in human cancer cells (Hum. Gene Ther. 10:155-164, 1999). International Patent Publication WO 97/04104 describes the HK-II promoter and indicates that it may be usefully employed in the treatment of diseases including cancer and diabetes.

In a further step in the glycolytic pathway, G6P is converted to fructose-6-phosphate (F6P) which is then phosphorylated by ATP to give fructose 1,6-biphosphate, a reaction that is catalyzed by 6-phosphofructo-1-kinase (PFK-1), a tetrameric enzyme. Separate genes encode a muscle subunit (M) and a liver subunit (L). PFK from muscle (PFKM) is a homotetramer of M subunits and PFK from liver (PFKL) is a homotetramer of L-subunits. PFK from platelets can be composed of any tetrameric combination of M and L subunits.

The enzyme 6-phosphofructo-2-kinase (PFK-2; also known as fructose-2,6-biphosphatase) is a bifunctional enzyme that is a critical regulator of glycolysis. When PFK-2 is phosphorylated it is inactive. However, in its non-phosphorylated form, PFK-2 catalyzes the synthesis of fructose-2,6-biphosphate by phosphorylating fructose-6-phosphate. The production of fructose-2,6-biphosphate leads to upregulation of 6-phosphofructokinase-1 (PFK-1), which in turn leads to stimulation of glycolysis and inhibition of the gluconeogenesis pathway.

There are four PFK-2 isoenzymes in mammals, each encoded by a different gene (referred to as pjkjb1-4) that expresses several isoforms of each isoenzyme. Regulatory sequences have been identified in these genes that account for their long-term control by hormones and tissue-specific transcription factors. The pjkjb3 gene product is constitutively expressed in proliferating tissue, transformed cell lines and in various tumors, being induced by hypoxia. Transcriptional regulation of the pjkjb3 promoter by HIF-1 has been demonstrated (Obach et al., J. Biol. Chem. 279:53562-53570, 2004).

As discussed above, in the process of aerobic respiration in normal cells, pyruvate is converted to acetyl-coenzyme A, which then enters into the Krebs cycle, providing ATP to the cell. This reaction is catalyzed by the enzyme pyruvate dehydrogenase (PDH), whose activity is under the control of pyruvate dehydrogenase kinases (PDKs). However, under hypoxic conditions, pyruvate is converted to lactate in a reaction catalyzed by lactate dehydrogenase 5 (LDH-5). In cancer cells, pyruvate is transformed to lactate regardless of the presence of oxygen (aerobic glycolysis). This reaction is controlled by the transcription factor HIF-1, which targets the ldh-a gene to produce LDH-5. In contrast to cancer cells, fibroblasts in the tumor supporting stroma exhibit an intense PDH but reduced PDK expression, thereby favoring maximum PDH activity. This means that stroma may use lactic acid produced by tumor cells, preventing the creation of an intolerable intra-tumoral acidic environment at the same time (Koukourakis et al., Neoplasia 7:1-6, 2005).

The proximal promoter of the gene encoding LDH contains binding sites for carbohydrate-responsive elements (ChoREs) and HIF-1α. LDH, which are tetramers (from peptide chains) of two 35 kDa units, are often up-regulated in tumors. The four peptide chains of LDH consist of two types, each under separate genetic control. The M and H subunits (or peptide chains) are so called because of their predominance in the muscle and heart. Five different isoenzymes (LDH-1-LDH-5) with different biochemical and physiological properties catalyze the same biochemical reaction, but differ in molecular structure and are generally organ specific. The LDH-5 isoenzyme consists of four M-chains and is the product of the ldh-a gene, while LDH-1 consists of four H chains, LDH-2 consists of three H and one M chains, LDH-3 consists of two H and two M chains, and LDH-4 consists of one H and three M chains.

LDH-5 catalyzes the conversion of pyruvate to lactate under anaerobic oxidation, but this function of LDH gradually fades away as the number of H over M chains increases. The LDH-1 isoenzyme favors aerobic oxidation of pyruvate by pyruvate dehydrogenase. LDH-1 was found to be consistently expressed in all living cells, normal and malignant, epithelial and stromal, including endothelium and lymphocytes. In contrast, LDH-5 was found to be expressed preferentially in tumor cells, while normal tissues were either devoid of LDH-5 or expressed it only faintly, with the cellular population of the tumor-supporting stroma showing LDH-5 activity in a small percentage of cases. LDH-5-positive stromal cells were associated with HIF-1α overexpression (Koukourakis et al., Tumour Biol. 24:199-202, 2003).

Most of the glucose catabolized by mammalian tissues proceeds down the glycolytic pathway discussed above. However there are other, more minor, metabolic pathways that are taken by glucose, one of which is the pentose phosphate pathway. This pathway generates NADPH and ribose 5-phosphate in the cytosol. NADPH, in contrast to NADH, is used in reductive biosynthesis, with ribose 5-phosphate being used in the synthesis of RNA, DNA and nucleotide coenzymes. The pentose phosphate pathway starts with the dehydrogenation of glucose 6-phosphate (G6P) by glucose 6-phosphate dehydrogenase (G6PDH), also known as hexose 6-phosphate dehydrogenase (H6PDH), to form a lactone, which is hydrolyzed to give 6-phosphogluconate and then oxidatively decarboxylated to yield ribulose 5-phosphate. NADP⁺ is the electron acceptor in both of these oxidations. The last step is the isomerization of ribulose 5-phosphate (a ketose) to ribose 5-phosphate (an aldose). Ribose-5-phosphate, a five carbon sugar, and its derivatives are components of ATP, CoA, NAD⁺, FAD, RNA and DNA.

There is a fundamental difference between NADPH and NADH. NADH is oxidized by the respiratory chain to yield ATP, whereas NADPH serves as an electron donor in reductive biosynthesis. Imatinib mesylate (ST1571; also known as Gleevec™, Novartis Pharmaceutical Corp., East Hanover, N.J.) and Genistein (4′,5,7-trihydroxy-isoflavone) are both tyrosine kinase inhibitors, which act by targeting HK and G6PDH and inhibiting their use as a substrate for carbons.

Defects in mitochondrial DNA have been found in many cancers which render the mitochondria respiratory deficient. As a result, in these cells energy production shifts to the glycolytic pathway, with a higher production of reactive oxygen species (ROS). Many tumor cells exhibit increased expression of most of the glycolytic enzymes which is paralleled by a decreased expression of several mitochondrial enzymes. Tumors with high glycolytic rates appear to often have significantly reduced mitochondrial content. Respiratory-deficient mitochondria are resistant to hypoxia-induced apoptosis. Certain mitochondrial proteins are released into the cytosol (for example, cytochrome C, endonuclease G) which decrease the rate of apoptosis in tumor cells and appear to be involved in the development of anticancer drug resistance.

The oxidative-phosphorylation system of mitochondria consists of a series of five major membrane complexes: NADH-ubiquinone oxidoreductase (commonly known as complex I); succinate-ubiquinone oxidoreductase (complex II); ubiquinol-cytochrome c oxidoreductase (cytochrome bc1 complex or complex III); cytochrome c-O₂ oxidoreductase (complex IV); and F1F0-ATP synthase (complex V). Parts of the respiratory complexes I and III-V are encoded by mitochondrial DNA (mtDNA), with only complex II being exclusively encoded by nuclear DNA (nDNA). The mutation rate in the mitochondrial genome is two orders of magnitude higher than that in the nuclear genome. This is due to: (1) mtDNA being in close vicinity to the electron transport chain with its ability to generate harmful reactive oxygen species (ROS); (2) limited repair capabilities of mitochondria; and (3) missing protection of mtDNA by histones and the chromatin structure.

Because mtDNA contains no introns, most mitochondrial mutations occur in coding regions. Mitochondrial genomic aberrations have been reported for solid tumors of the breast, colon, stomach, liver, kidney, bladder, lung, head, neck and blood. The generation of ROS in mitochondria, which typically occurs under hypoxic ATP depletion, can be considered a major cause of aberrant mtDNA in most of these tumors. Although the mechanisms of interaction between mtDNA and nDNA are still largely unknown, it has been shown that parts of the mtDNA, including mutated elements, can be incorporated into the nDNA. One of the consequences of such perturbations in the respiratory enzymes is an accumulation of reduced nicotinamide adenine dinucleotide (NADH) in the mitochondria with a subsequent elevation of pyruvate and ultimately lactate in the cytosol.

### Energy Pathways in Cancer Cells

Living organisms require a continual input of free energy for three major purposes: the synthesis of macromolecules from simple precursors; the active transport of molecules and ions; and the performance of mechanical work, such as muscle contraction. In mammals, this energy is obtained by the oxidation of foodstuffs. Part of the free energy from this process is transformed into a highly accessible form before it is used for the three major purposes described above. The free energy donor in most energy-requiring processes is the nucleotide adenosine triphosphate (ATP). ATP is an energy-rich molecule because its triphosphate moiety contains two phosphoanhydride bonds. A large amount of energy is generated when ATP is hydrolyzed to adenosine diphosphate (ADP) and orthophosphate, or when ATP is hydrolyzed to adenosine monophosphate (AMP) and pyrophosphate.

Other nucleotides, or nucleotide derivatives, also play pivotal roles in energy metabolism, and in many different biochemical reactions. For example, the nucleotide guanosine 5′-triphosphate (GTP) powers many movements of macromolecules, such as the translocation of nascent peptide chains or ribosomes and the activation of signal-coupling proteins. Adenine nucleotides are structural components of the three major energy process co-enzymes nicotinamide adenine dinucleotide (NAD⁺), flavin adenine dinucleotide (FAD) and coenzyme A (CoA). A common feature of the biosynthesis of NAD⁺, FAD and CoA is the transfer of the AMP moiety to the phosphate group of a phosphorylated intermediate. The resulting pyrophosphate is then hydrolyzed to orthophosphate. Much of the thermodynamic driving force comes from the hydrolysis of the released pyrophosphate. ATP, NAD⁺, FAD⁺ and CoA are not the only key energy generating molecules. However, the inhibition of the formation of ATP, NADH, FADH or acetyl CoA will each, separately, result in cell death.

Nucleotides are the activated precursors of DNA and RNA, and nucleotide derivatives are activated intermediates in many biosyntheses. For example, UDP-glucose and CDP-diacylglycerol are precursors of glycogen and phosphoglycerides, respectively. S-adenosylmethionine is used to introduce a methyl group in the course of several synthetic pathways. Nucleotides also serve as metabolic regulators. For example, AMP is a ubiquitous mediator of the action of many hormones. Covalent modifications introduced by ATP alter the activities of many enzymes, as exemplified by the phosphorylation of glycogen synthase and the adenylation of glutamine synthetase.

ATP, ADP and AMP are interconvertible, with the interconversion being catalyzed by the enzyme adenylate kinase in the cytosol and the enzyme adenylate synthase in mitochondria. In humans, seven human adenylate kinase family members have been identified, with some of these family members having a number of isomers. Genetic ablation of adenylate kinase 1 (AK1) disturbs muscle energetic economy and decreases tolerance to metabolic stress, despite rearrangements in alternative high energy phosphoryl transfer pathways (Janssen et al., J. Biol. Chem. 278:12937-12945, 2003).

In aerobic organisms, while some ATP is generated by the glycolytic pathway as glucose is degraded to produce building blocks for biosynthetic reactions, the major source of ATP is the process called oxidative phosphorylation. Oxidative phosphorylation generates 26 of the 30 molecules of ATP that are formed when glucose is completely oxidized to CO₂ and H₂O.

The glycolytic pathway is the prelude to both the citric acid cycle and oxidative phosphorylation. The glycolytic pathway degrades glucose to pyruvate in the cytosol. Pyruvate then enters the mitochondria to fuel the citric acid cycle and oxidative phosphorylation. Oxidative phosphorylation is the enzymatic phosphorylation of ADP to form ATP, coupled to electron transport from a substrate to molecular oxygen.

More specifically, in aerobic organisms free energy is derived from the oxidation of fuel molecules and the transfer of electrons to the ultimate electron acceptor, oxygen (O₂). Electrons are not transferred from fuel molecules directly to O₂, rather they are transferred from fuel molecules to special carriers which are either a pyridine nucleotide, namely NAD⁺, or a flavin, namely FAD. This results in the intermediates NADH and FADH₂, which are reduced forms of pyridine nucleotides and flavins, respectively. These intermediates then transfer their high potential electrons to O₂ by means of an electron transport chain in mitochondria. The proton gradient formed as a result of this flow of electrons drives the synthesis of ATP from ADP and inorganic phosphate (Pi). This process is called oxidative phosphorylation.

In oxidative phosphorylation, the synthesis of ATP is coupled to the flow of electrons from NADH or FADH₂ to O₂ by a proton gradient across the inner mitochondrial membrane. Electron flow through three asymmetrically oriented transmembrane complexes results in the pumping of protons out of the mitochondrial matrix and the generation of a membrane potential. Electron transport is normally tightly coupled to phosphorylation. NADH and FADH₂ are oxidized only if ADP is simultaneously phosphorylated to ATP. This coupling, called respiratory control, can be disrupted by uncouplers such as 2,4-dinitrophenol (DNP), which dissipate the proton gradient by carrying protons across the inner mitochondrial membrane.

The electron carriers in the respiratory assembly of the inner mitochondrial membrane are flavins, iron-sulfur complexes, quinones, heme groups of cytochromes, and copper ions. Electrons from NADH are transferred to the flavin mononucleotide (FMN) prosthetic group of NADH-Q reductase, the first of three complexes. This reductase also contains Fe—S centers. NADH-Q reductase is a large enzyme of 880 kDa that consists of at least 34 polypeptide chains. The electrons emerge in Q11₂, the reduced form of ubiquinone (Q). This highly mobile hydrophobic carrier transfers its electrons to cytochrome reductase, a complex that contains cytochromes b and c and an Fe—S center. This second complex reduces cytochrome c, a water-soluble peripheral membrane protein. Cytochrome c, like Q, is a mobile carrier of electrons, which it then transfers to the complex cytochrome oxidase. This third complex contains cytochromes a and a3, and two copper ions. A heme ion and a copper ion in this oxidase transfer electrons to O₂, the ultimate acceptor, to form H₂O. Succinate-Q reductase, in contrast with the other complexes, does not pump protons. NADH-Q reductase, succinate-Q reductase, cytochrome reductase, and cytochrome oxidase are also called Complex I, II, III, and IV, respectively.

The flow of electrons through each of these complexes leads to the pumping of protons from the matrix side to the cytosolic side of the inner mitochondrial membrane. A proton-motive force, consisting of a pH gradient (cytosolic side acidic) and a membrane potential (cytosolic side positive), is generated. The flow of protons back to the matrix side through ATP synthase drives ATP synthesis. The enzyme complex ATP synthase consists of a hydrophobic F₀ unit that conducts protons through the membrane and a hydrophilic F₁ unit that catalyzes ATP synthesis sequentially at three sites. Protons flowing through ATP synthase release tightly bound ATP.

ATP and ADP do not diffuse freely across the inner mitochondrial membrane. Rather, a specific transport protein, ATP-ADP translocase (also called the adenine nucleotide carrier), enables these charged molecules to traverse this permeability barrier. The flows of ATP and ADP are coupled: ADP enters the mitochondrial matrix only if ATP exits, and vice versa. This reaction is catalyzed by a translocase. The enzyme ATP-ADP translocase, a dimer of identical 30 kDa subunits, contains a single nucleotide binding site that alternately faces the matrix and cytosolic sides of the membrane. ATP and ADP (both devoid of Mg²⁺) are bound with nearly the same affinity. In the presence of a positive membrane potential, the rate of binding-site release from the matrix to the cytosolic side is more rapid for ATP than for ADP because ATP has one more negative charge. Hence, ATP is transported out of the matrix about 30 times more rapidly than is ADP, which leads to a higher phosphoryl potential on the outer side than on the matrix side. The translocase does not evert at an appreciable rate unless a nucleotide is bound. This assures that the entry of ADP into the matrix is precisely coupled to the exit of ATP. On the other hand, the membrane potential is decreased by the exchange of ATP for ADP, which results in a net transfer of one negative charge out of the matrix. ATP-ADP exchange is energetically expensive; about a quarter of the energy yield from electron transfer by the respiratory chain is consumed to regenerate the membrane potential that is tapped by this exchange process.

The translocase is highly abundant, constituting about 14% of the inner mitochondrial membrane protein. It is specifically inhibited by very low concentrations of atractyloside, a plant glycoside, or of bongkrekic acid, an antibiotic from a mold. Atractyloside binds to the translocase when its nucleotide site faces outward, whereas bongkrekic acid binds when this site faces the matrix. Oxidative phosphorylation stops soon after either inhibitor is added, showing that the ATP-ADP translocase is essential for this process.

In the synthesis of purine nucleotides, the purine ring is assembled from a variety of precursors: glutamine, glycine, aspartate, methylenetetrahydrofolate, N¹⁰-formyl-tetrahydrofolate and CO₂. The committed step in the de novo synthesis of purine nucleotides is the formation of 5-phosphoribosylamine from 5-phosphoribosyl-1-pyrophosphate (PRPP) and glutamine, which is catalyzed by amido-phosphoribosyltransferase. PRPP is synthesized from ATP and ribose 5-phosphate in a reaction catalyzed by PRPP synthetase. The purine ring is assembled on ribose phosphate. The addition of glycine, followed by formylation, amination and ring closure, yields 5-aminoimidazole ribonucleotide. This intermediate contains the completed five-membered ring of the purine skeleton. The addition of CO₂, the nitrogen atom of aspartate and a formyl group, followed by ring closure, yields inosine monophosphate (IMP), a purine ribonucleotide. IMP dehydrogenase 2 (IMPDH2) is the rate-limiting enzyme of de novo GTP biosynthesis and catalyzes the NAD-dependent oxidation of inosine-5′-monophosphate into xanthine-5′-monophosphate, which is then converted into guanosine-5′-monophosphate.

AMP and guanosine 5′-monophosphate (GMP) are formed from IMP. Purine ribonucleotides can also be synthesized by a salvage pathway in which a preformed base reacts directly with PRPP.

In the synthesis of pyrimidine nucleotides, the pyrimidine ring is assembled first and then linked to ribose phosphate to form a pyrimidine nucleotide, in contrast with the sequence in the de novo synthesis of purine nucleotides. PRPP is again the donor of the ribose phosphate moiety. In the first step, carbamoyl phosphate is formed in an ATP-dependent reaction catalyzed by the enzyme carbamoyl phosphate synthetase. The synthesis of the pyrimidine ring starts with the formation of carbamoylaspartate from carbamoyl phosphate and aspartate, a reaction catalyzed by aspartate transcarbamoylase. Dehydration, and cyclization, which is catalyzed by dihydroorotase, is followed by oxidation which yields orotate, which in turn reacts with PRPP to give orotidylate. Decarboxylation of this pyrimidine nucleotide yields uridine 5′-monophosphate (UMP). CTP synthetase (CTPS) catalyzes the amination of UTP, the last step in CTP biosynthesis, to give cytidine 5′-triphosphate (CTP). The first three enzymes involved in this process, carbamoyl phosphate synthetase, aspartate transcarbamoylase and dihydroorotase, are covalently joined in a single 240 kDa polypeptide chain called CAD.

The bifunctional enzyme uridine monophosphate synthase (uridine 5′-phosphate synthase; UMPS) catalyzes the last two steps in de novo pyrimidine biosynthesis. UMPS contains two sequential catalytic activities for the synthesis of orotidine 5′-phosphate (OMP) from orotate (orotate phosphoribosyltransferase or orotidine-5′-phosphate:pyrophosphate phosphoribosyltransferase (OPRT)) and the decarboxylation of OMP to form UMP (OMP decarboxylase or orotidine-5′-phosphate carboxy-lyase) (Traut, Arch. Biochem Biophys. 268:108-115, 1989; Suttle et al., Proc. Natl. Acad. Sci. USA 185:1754-1758. 1988).

Deoxyribonucleotides, the precursors of DNA, are formed by the reduction of ribonucleoside diphosphates in the de novo synthesis pathway. These conversions are catalyzed by ribonucleotide reductase. This enzyme consists of two subunits, B₁ (RRM1, a 172 kDa dimer) and B₂ (RRM2, an 87 kDa dimer). The reduction of ribonucleotides to deoxyribonucleotides is precisely controlled by allosteric interactions of these two subunits. Electrons are transferred from NADPH to sulfhydryl groups at the active sites of this enzyme by thioredoxin or glutaredoxin. A tyrosyl free radical that is generated by an iron center in the reductase participates in catalyzing the exchange of H for OH at C-2 of the sugar unit. dTMP is formed by methylation of dUMP, which is catalyzed by thymidylate synthetase. Inhibition of thymidylate synthetase stops DNA production, arresting the cell cycle and eventually leading to “thymineless” cell death. The methylation of dUMP is the reaction that is targeted by the cancer drug 5-fluorouracil. 5-Fluorouracil is an analog of dUMP and irreversibly inhibits thymidylate synthetase after acting as a normal substrate. High levels of thymidylate synthetase have been correlated with poor prognosis in patients with colorectal, breast, cervical, bladder, kidney and non-small cell lung cancers. Thymidylate synthetase also exhibits oncogene-like activity, suggesting a link between thymidylate synthetase-regulated DNA synthesis and the induction of a neoplastic phenotype (Rahman et al., Cancer Cell 5:301-302, 2004). Schmitz et al. (Cancer Research 64:1431-1435, 2004) have demonstrated that siRNA molecules can be used to inhibit thymidylate synthetase expression, and suggested that such molecules may have therapeutic potential either alone or as chemosensitizers in combination with thymidylate synthetase inhibitor compounds.

Deoxyribonucleotides can also be synthesized via phosphorylation of deoxyribonucleosides by deoxyribonucleoside kinases in the salvage pathway (Reichard, Annu. Rev. Biochem. 57:349-374, 1998). There are four distinct human deoxyribonucleoside kinases. Deoxycytidine kinase (dCK), deoxyguanosine kinase (dGUOK), and thymidine kinase 2 (TK2) are constitutively expressed throughout the cell cycle and are closely related members of the enzyme family, while thymidine kinase 1 (TK1) is not sequence-related to the other enzymes and is strictly S-phase-regulated (Amer and Eriksson, Pharmacol Ther. 67:155-186, 1995). Deoxycytidine kinase (dCK) catalyzes the rate-limiting step of the deoxynucleoside salvage pathway in mammalian cells and plays a key role in the activation of several pharmacologically important nucleoside analogs. dCK also phosphorylates clinically important anticancer and antiviral nucleoside analogs in addition to deoxycytidine, deoxyadenosine, and deoxyguanosine (Johansson et al., Proc. Natl. Acad. Sci. USA 94:11941-11945, 1997). The nucleoside analogs phosphorylated by dCK include 1-β-D-arabinofuranosylcytosine (araC), 9-β-D-arabinofuranosyladenine, and 2-chloro-2′-deoxyadenosine (CdA), which are commonly used in the treatment of hematological malignancies (Tallman and Hakimian, Blood 86:2463-2474, 1995), and 2′,2′-difluorodeoxycytidine (dFdC), which is active against several solid malignant tumors (Hui and Reitz, Am. J. Health-Syst. Pharm. 54:162-170, 1997).

DGUOK and TK2 are mitochondrial deoxyribonucleoside kinases. DGUOK phosphorylates purine deoxyribonucleosides in the mitochondrial matrix and TK2 phosphorylates deoxythymidine, deoxycytidine, and deoxyuridine. In addition, DGUOK and TK2 phosphorylate several nucleoside analogs in vitro (Wang et al., J. Biol. Chem. 268:22847-22852, 1993; Munch-Petersen et al., J. Biol. Chem. 266:9032-9038, 1991).

Thymidine kinase 1 (TK1) phosphorylates only deoxythymidine and deoxyuridine, and is strictly cell-cycle-regulated. TK1 activity is low or absent in resting cells, starts to occur in late G₁ cells, increases in S phase (coinciding with the increase in DNA synthesis), and disappears during mitosis (Welin et al., Proc. Natl. Acad. Sci. USA 101: 17970-17975, 2004).

The one-carbon and electron donor in the conversion of dUMP to dTMP is tetrahydrofolate, which is converted into dihydrofolate. Tetrahydrofolate is subsequently regenerated by the reduction of dihydrofolate by NADPH. Dihydrofolate reductase, which catalyzes the conversion of tetrahydrofolate to dihydrofolate, is inhibited by dihydrofolate analogs, such as the cancer drugs aminopterin and methotrexate. Mammalian tumor cells can become resistant to methotrexate. Methotrexate interferes with DNA synthesis and repair, and cellular replication, resulting in fewer tetrahydrofolate derivatives in the cell so that purine synthesis is blocked.

In humans, purines are degraded to urate. Gout, a disease that affects joints and leads to arthritis, is associated with excessive production of urate. Allopurinol, a suicide inhibitor of xanthine oxidase, is used to treat gout. This analog of hypoxanthine blocks the formation of urate from hypoxanthine and xanthine. Lesch-Nyhan syndrome, a genetic disease characterized by self-mutilation, mental deficiency and gout, is caused by the absence of hypoxanthine-guanine phosphoribosyl transferase. This enzyme is essential for the synthesis of purine nucleotides by the salvage pathway.

The first step in the synthesis of nicotinamide adenine dinucleotide (NAD⁺) is the formation of nicotinate ribonucleotide from nicotinate and PRPP. Nicotinate (also called niacin) is derived from tryptophan. Humans can synthesize the required amount of nicotinate if the supply of tryptophan in the diet is adequate. However, an exogenous supply of nicotinate is required if the dietary intake of tryptophan is low. A dietary deficiency of tryptophan and nicotinate can lead to Pellagra, a disease characterized by dermatitis, diarrhea and dementia.

Following the formation of nicotinate ribonucleotide, an AMP moiety is transferred from ATP to nicotinate ribonucleotide to form desamido-NAD⁺. The final step is the transfer of the amide group of glutamine to the nicotinate carboxyl group to form NAD⁺. NADP⁺ is then derived from NAD⁺ by phosphorylation of the 2′-hydroxyl group of the adenine ribose moiety. This transfer of a phosphoryl group from ATP is catalyzed by NAD⁺ kinase.

Flavin adenine dinucleotide (FAD) is synthesized from riboflavin and two molecules of ATP. First riboflavin is phosphorylated by ATP to give riboflavin 5-phosphate (also called flavin mononucleotide). FAD is then formed by the transfer of an AMP moiety from a second molecule of ATP to riboflavin 5′-phosphate.

The AMP moiety of coenzyme A also comes from ATP. A common feature of the biosyntheses of NAD⁺, FAD and CoA is the transfer of the AMP moiety of ATP to the phosphate group of a phosphorylated intermediate. The pyrophosphate formed in these condensations is then hydrolyzed to orthophosphate. As in many other biosyntheses, much of the thermodynamic driving force comes from the hydrolysis of the released pyrophosphate.

In mammalian cells, fatty acids represent a major form of stored energy. Excess dietary carbohydrate is converted to fatty acid and stored in adipose tissue as triglyceride, together with excess dietary fat. The enzyme responsible for the conversion of carbohydrates into fatty acids is fatty acid synthase (FASN). FASN functions as a homodimer consisting of seven distinct catalytic centers arranged around a central acyl carrier protein. Its main function is to catalyze the synthesis of palmitate from acetyl-CoA and malonyl-CoA, in the presence of NADPH, into long-chain saturated fatty acids.

ATP citrate-lyase (ACLY) is the primary enzyme responsible for the synthesis of cytosolic acetyl-CoA in many tissues and has a central role in de novo lipid synthesis. The enzyme is a 40 kDa tetramer of identical subunits and catalyzes the formation of acetyl-CoA and oxaloacetate from citrate and CoA with a concomitant hydrolysis of ATP to ADP and phosphate. The product, acetyl-CoA, serves several important biosynthetic pathways, including lipogenesis and cholesterogenesis. In nervous tissue, ATP citrate-lyase may be involved in the biosynthesis of acetylcholine. Two transcript variants encoding distinct isoforms have been identified for this gene.

### Role of Transporters in Cancer

Normal cells employ a number of transport systems in a range of important cellular functions. The transporters provide nutrients, remove unwanted materials, establish electrochemical gradients across membranes, and affect entry and extrusion of drugs into and from cells (Lee, Eur. J. Pharm. Sci. 11 (Suppl. 2):S41-50, 2000; FIG. 3). The main characteristics that distinguish cancer cells from normal cells are their unfettered growth, and an increased and altered metabolism (Fuchs and Bode, Semin. Cancer Biol. 15:254-266, 2005). To support this increase in growth and metabolism, tumor cells absorb large quantities of nutrients from their surroundings. In addition to glucose and other energy sources, tumor cells are also voracious in their consumption of amino acids, nucleosides and water-soluble vitamins.

The cellular import of these solutes is mediated by the solute carrier (SLC) superfamily, a group of over 300 proteins transporting a large variety of nutrients, including organic nutrients, inorganic ions, drugs and their metabolites (Hediger, Eur. J. Physiol. 447:465-468, 2004). A number of SLC proteins have been shown to play a role in cancer cell biology. SLC proteins are involved in provision of essential nutrients, mediation of the uptake of antimetabolite drugs and modulation of cellular physiology. As a result, a number of reports have shown that SLC transporters influence the chemoresistance and chemosensitivity of cells to anticancer drugs (see FIG. 3). For example, expression of the reduced folate carrier (RFC or SLC19A1) correlates with methotrexate resistance (Ganaphthy, Eur. J. Physiol. 447:641-646, 2004), and loss of uptake mediated by RFC is a well-known mechanism of MTX resistance. Likewise, the fact that the well-known ENT (SLC29) family of facilitative nucleoside transporters correlate with sensitivity to nucleoside analogue drugs, fits with their ability to transport both normal nucleosides and their analogues into cells. In fact, small molecule inhibitors of nucleoside transport have been tested as chemosensitizers but, although effective in vitro, clinical trials have been unsuccessful. These proteins, for which there are as yet no effective drug treatments, represent ideal targets for RNAi-based therapeutics.

In contrast to the examples above, some transporters correlate with drug sensitivity/resistance profiles in more unexpected ways; for example, expression of a mitochondrial amino acid transporter and a number of ion pumps are associated with resistance to platinum drugs (Huang, Cancer Res. 64:4294-4301, 2004).

Amino acid transporters also modulate chemoresistance/sensitivity in cancer cells. ASCT2, a glutamine transporter, is expressed in all tumor cells and is necessary for the growth of cell lines in culture, implicating a role for this transporter in the immortalization process (Fuchs and Bode, Sem. Cancer Biol. 15:254-266, 2005). Another amino acid transporter, xCT (SLC7A11), modulates chemoresistance by controlling the uptake of cysteine and thereby intracellular levels of reduced glutathione, an important component of phase II metabolism (Huang, Cancer Res. 64:4294-4301, 2004). It has been demonstrated that siRNA-mediated knockdown of this transporter affected drug potencies similarly to small molecule transport inhibitors (Huang, Cancer Res. 65:7446-7454, 2005).

In addition to numerous solute carriers, tumor cells also express ATP-dependent pumps, several of which limit the accumulation of anticancer drugs inside cells—so-called classical multidrug resistance (MDR; see FIG. 3). This phenomenon is characterized by the emergence of cells resistant to multiple structurally and functionally unrelated drugs following exposure to a single agent, and is associated with the upregulation of drug pumps in the cytoplasmic membrane. In one of the first uses of RNAi in cancer biology, MDR was successfully reversed using siRNAs targeting MDR1, the major determinant of clinical MDR (Wu, Cancer Res. 63:1515-1519, 2003).

### RNA Interference

The treatment methods disclosed herein utilize the process of RNA interference (RNAi). RNAi is a post-transcriptional RNA silencing phenomenon used by most eukaryotic organisms as a defense mechanism against viral attack and transposable factors. This RNA silencing process was first identified in plants, where it is referred to as post-transcriptional gene silencing (PTGS), and was subsequently observed in the nematode C. elegans by Fire et al. (Nature 391:806-811, 1998). RNAi involves the use of small interfering nucleic acid or RNA molecules (siRNAs) that selectively bind with complementary mRNA sequences, targeting them for degradation and thus inhibiting corresponding protein production. More recently it has been shown that siRNAs can induce de novo methylation and silencing of promoter sequences, known as transcriptional gene silencing (TGS).

More specifically, in an initiation step double-stranded RNA (dsRNA) is digested by the enzyme Dicer (a member of the RNase III family of dsRNA-specific ribonucleases) into small interfering RNAs (siRNAs) of 19-25 nucleotides in length. Each siRNA consists of two separate, annealed single strands of nucleotides. Each strand may have a 2-3 nucleotide 3′ overhang. In the effector step, siRNA duplexes bind to a nuclease complex to form an RNA-induced silencing complex (RISC). The RISC then targets the endogenous mRNA complementary to the siRNA within the complex, and cleaves the endogenous mRNA approximately twelve nucleotides from the 3′ terminus of the siRNA. Degradation of the endogenous mRNA is then completed by exonucleases. An amplification step may also exist within the RNAi pathway in some organisms, for example by copying of the input dsRNAs or by replication of the siRNAs themselves.

Transfection of long dsRNA molecules of greater than 30 nucleotides into most mammalian cells causes nonspecific suppression of gene expression, as opposed to the gene-specific suppression seen in non-mammalian organisms. This is believed to be due to activation of an antiviral defense mechanism that includes the production of interferon, and that leads to a global shut-down of protein production. However it has been shown that this pathway is not activated by dsRNAs less than 30 nucleotides in length, and that short dsRNAs of 21-23 nucleotides can be used to reduce specific gene expression in mammalian cells (Caplen et al., Proc. Natl. Acad. Sci. USA 17:9742-9747, 2001; Elbashir et al., Nature 6836:494-498, 2001). Brummelkamp et al. have demonstrated that siRNAs targeting oncogenes are effective in reducing tumors in mice (Cancer Cell 2:243-247, 2002).

RNAi has several advantages over other gene silencing techniques, such as the use of antisense oligonucleotides (ODN). RNAi techniques result in more specific inhibition of gene expression than ODN and are able to induce the same level of silencing as ODN at much lower concentrations of reagent. Also, siRNAs are more resistant to nuclease degradation than ODN. Bertrand et al. (Biochem. Biophys. Res. Commun. 296:1000-1004, 2002) have shown that, in mice, siRNA silencing is more effective than antisense suppression.

It has been shown that sequence specificity of siRNA is important, as single base pair mismatches between the siRNA and its target mRNA can dramatically reduce silencing. Accordingly, in order to be effective in suppressing expression of a gene of interest to a high degree, siRNAs must be designed so that they are specific to the target gene. In addition, in order to avoid unwanted side effects, a delivery system must be employed that specifically delivers the siRNA to the desired target. Delivery of siRNA to cells by means of exogenous delivery of preformed siRNAs or via promoter-based expression of siRNAs or shRNAs has been described. Genetic constructs for the delivery of siRNA molecules are described, for example, in U.S. Pat. No. 6,573,099. The delivery of short RNA fragments to cells in vivo in mammals can be problematic due to the rapid degradation of the RNA. Short hairpin RNAs (shRNA) are nucleic acid molecules that mimic the structure of the RNAi duplex and that can be produced in cells following delivery of expression vectors encoding the shRNA. The use of shRNA expression plasmids to reduce gene expression in vivo in rats has been described by Zhang et al. (J. Gene Med. 5:1039-1045, 2003).

International patent publication no. WO 2005/032486 describes an adenovirus vector that encodes an siRNA directed against the HIF-1α gene and its use to inhibit tumor growth in a mouse model. Yu et al. have described studies employing RNA interference targeted against HIF-1α mRNA to evaluate the role of HIF-1α in apoptosis in primary cultured human endothelial cells (Lab. Invest. 84:553-561, 2004).

### SUMMARY OF THE INVENTION

Briefly stated, the present invention provides compositions for the treatment and/or prevention of a cancer in a mammal by means of RNA interference, together with methods for the use of such compositions. The cellular targets for siNA employed in the compositions are: genes that encode enzymes involved in the metabolism of glucose, more specifically in the glycolysis pathway; genes that encode specific enzymes involved in the biosynthesis of nucleotides or in glycogen metabolism; genes that encode electron carriers that work with ATP to provide metabolic energy; and/or genes that encode proteins involved in the transport of amino acids, peptides, sugars, monocarboxylic acids, organic cations, phosphates, nucleosides and/or water-soluble vitamins.

The disclosed compositions comprise at least one small interfering nucleic acid molecule (siNA) that suppresses expression of a target gene within a target cell. In one embodiment, the disclosed compositions comprise: (a) a binding agent that specifically binds to a target internalizable cell surface molecule, or antigen, that is expressed on the surface of a target cell of interest, and (b) a nucleic acid binding component, normally a cationic polymer or cationic lipid, to condense and protect the siNA, and (c) at least one siNA that suppresses expression of a target gene within the target cell, whereby, after binding to the target cell surface molecule, the binding agent and siNA are internalized into the cell, and the siNA released. In one embodiment, the composition comprises a cationic lipid or cationic polymer covalently linked to a folic acid molecule to target the folate receptor on cancer cells.

In related embodiments, the disclosed compositions comprise: (a) a binding agent that specifically binds to a target internalizable cell surface molecule, or antigen, that is expressed on the surface of a target cell of interest; and (b) a genetic construct that is capable of expressing at least one siNA that suppresses expression of a target gene within the target cell, whereby, after binding to the target cell surface molecule, the binding agent and genetic construct are internalized into the cell, and the siNA is expressed by the genetic construct. In a further aspect, the compositions comprise a genetic construct that is capable of expressing a siNA that suppresses expression of a target gene within the target cell, wherein the genetic construct is packaged within a viral vector which, upon infection of the cell, releases its genetic material enabling expression of the genetic construct. Preferably the viral vector is an adenovirus-associated vector (AAV). In this aspect, viral capsid proteins may act as a binding agent.

In certain embodiments, compositions are provided that comprise a genetic construct that expresses a siNA, wherein expression of the siNA is under the control of a promoter or promoter region that is specific to the target cell, so that suppression of gene expression in non-target cells is reduced. For example, promoters that drive the expression of HIF-1 or HK-II, or promoter regions containing response elements that are involved in regulating transcription of HIF-1 or HK-II, may be employed to prevent expression of the siNA in non-tumor cells. Other promoters that may be effectively employed in the disclosed genetic constructs include those for c-erbB-2 (also known as HER-2/neu), prostate-specific antigen (PSA), osteocalin, clusterin, EIA (human adenovirus type 5 early region 1A) and survivin. Alternatively, the siNA may be under the control of an RNA polymerase III or a tissue-specific RNA polymerase II promoter.

In certain embodiments, the binding agent employed in the compositions is an antibody, or an antigen-binding fragment thereof. Other binding agents that may be effectively employed in the inventive compositions include cell-specific ligands, and peptides or small molecules that specifically bind to cell-specific receptors. Viral (capsid) proteins may also be employed as binding agents.

In one embodiment, the binding agent is linked to the siNA, genetic construct or viral vector by means of a streptavidin-biotin linker as described below. In another embodiment, the siNA, genetic construct or viral vector is complexed to a lipid carrier, such as a cationic or anionic lipid carrier, which in turn is linked to the binding agent. In a related embodiment, the siNA, genetic construct or viral vector is encapsulated within a liposome, and the binding agent, or the antigen-binding portion thereof, is present on the surface of the liposome.

In certain of the disclosed compositions, the target cell surface molecule is an internalizable molecule that is expressed on the surface of a tumor cell, wherein binding of a complex to the cell surface molecule leads to internalization of the complex within the tumor cell. In certain embodiments the target cell surface molecule is selected from the group consisting of the receptors for: transferrin; endothelin I; and VEGF, including the VEGF165b isomer.

The compositions disclosed herein are effective in reducing expression of at least one gene that is involved in the development and/or progression of a cancer, Examples of such genes include: genes that are active in an energy metabolism pathway, biosynthesis of nucleotides or in glycogen metabolism; genes that encode electron carriers that work with ATP to provide metabolic energy; and genes that encode proteins involved in the transport of amino acids, peptides, sugars, monocarboxylic acids, organic cations, phosphates, nucleosides and/or water-soluble vitamins. In certain embodiments, the siNA employed in the compositions is capable of suppressing production of HIF-1 (preferably HIF-1α and/or HIF-β, or a splice variant thereof); HK-II; LDH-5; G6P dehydrogenase; Pfkfb3; PFKL; 5-phosphoribosyl-1-pyrophosphate synthetase (PRPS); amido-phosphoribosyltransferase (PPAT); carbamoyl phosphate synthetase 1 (CPS1); carbamoyl phosphate synthetase 2 (CAD); ribonucleotide reductase subunit 1 (RRM1); ribonucleotide reductase subunit 2 (RRM2); ribonucleotide reductase subunit 2B (RRM2B); thymidylate synthetase (TYMS); dihydrofolate reductase (DHFR); adenylate kinase (AK1); NAD synthase (NADSYN); flavin adenine dinucleotide synthase (FLAD1); NADH-Q reductase (NADHQ); cytochrome reductase (UQCRC2); cytochrome oxidase (COX5a); ATP synthase (ATP5B); fatty acid synthase (FASN); ATP citrate lyase (ACLY); ATP-ADP translocase 1 to 3 (ANT1, ANT2, ANT3); CTP synthase (CTPS); inosine monophosphate dehydrogenase 2 (IMPDH2); deoxycytidine kinase (DCK); thymidine kinase 1 (TK1); thymidine kinase 2 (TK2); deoxyguanosine kinse (DGUOK); uridine monophosphate synthetase (UMPS); or dihydropyrimidine dehydrogenase (DPYD).

Examples of siNAs that are capable of suppressing expression of HIF-1α, HIF-β, HK-II, LDH-5, G6P dehydrogenase, PFKL or Pfkfb3 include the siRNA sequences corresponding to the target sequences provided in SEQ ID NO: 1 to 15 and 17. Examples of siNAs that are capable of suppressing expression of PRPP synthetase, amido-phosphoribosyltransferase (PPAT), CPS1, CAD, ribonucleotide reductase subunits 1 and 2, ribonucleotide reductase subunit 2B (RRM2B), thymidylate synthetase, dihydrofolate reductase, adenylate kinase, NADSYN, FAD synthase, NADH-Q reductase, cytochrome reductase, cytochrome oxidase, ATP synthase, FASN, ACLY, or ATP-ADP translocases 1 to 3, CTPS, IMPDH2, deoxycytidine kinase (DCK), thymidine kinase 1 (TK1), thymidine kinase 2 (TK2), deoxyguanosine kinse (DGUOK), uridine monophosphate synthetase (UMPS) and dihydropyrimidine dehydrogenase (DPYD) include the siRNA sequences corresponding to the target sequences provided in SEQ ID NO: 18-47.

In an alternative embodiment, the siNA employed in the disclosed compositions is targeted against the promoters for HIF-1α, HIF-β, HK-II, LDH-5, G6P dehydrogenase, PFKL, Pfkfb3, PRPP synthetase, amido-phosphoribosyltransferase (PPAT), CPS1, CAD, ribonucleotide reductase subunits 1 and 2, ribonucleotide reductase subunit 2B (RRM2B), thymidylate synthetase, dihydrofolate reductase, adenylate kinase, NADSYN, FLAD1, NADH-Q reductase, cytochrome reductase, cytochrome oxidase, ATP synthase, FASN, ACLY, ANT1, ANT2, ANT3, CTPS, IMPDH2, deoxycytidine kinase (DCK), thymidine kinase 1 (TK1), thymidine kinase 2 (TK2), deoxyguanosine kinse (DGUOK), uridine monophosphate synthetase (UMPS) or dihydropyrimidine dehydrogenase (DPYD) whereby introduction of the genetic construct into a target cell, such as a tumor cell, will lead to transcriptional gene silencing of the HIF-1α, HIF-β, HK-II, LDH-5, G6P dehydrogenase, PFKL, Pfkfb3, PRPP synthetase, PPAT, CPS1, CAD, ribonucleotide reductase subunits 1 and 2, ribonucleotide reductase subunit 2B (RRM2B), thymidylate synthetase, dihydrofolate reductase, adenylate kinase, NADSYN, FLAD1, NADH-Q reductase, cytochrome reductase, cytochrome oxidase, ATP synthase, FASN, ACLY, ANT1, ANT2, ANT3, CTPS, IMPDH2, DCK, TK1, TK2, DGUOK, UMPS or DPYD genes in the target cell.

In certain embodiments, the compositions disclosed herein are effective in reducing expression of a gene that is active in transport of metabolites involved in the development and/or progression of a cancer. In one aspect, the siNA employed in such compositions are capable of suppressing production of one or more transporters in the SLC2A subfamily of glucose transporters. The glucose transporter genes are expressed by every living cell, mediating the facilitative uptake of glucose down its concentration gradient. The inwardly directed glucose gradient is maintained by the rapid metabolism of glucose once inside the cell, effectively limiting free intracellular glucose. The SLC2A subfamily of glucose transporters consists of 13 members (Uldry and Thorens, Eur. J. Physiol. 447:480-489, 2004), which fall into three classes: Class I contains the glucose transporters GLUT1, GLUT2, GLUT 3 and GLUT4; Class II comprises the fructose transporter GLUT5 and the recently described GLUT7, GLUT9 and GLUT11; and Class III consists of GLUT6, GLUT 8, GLUT 10 and GLUT 12 plus HMIT (Joost, Am. J. Physiol. Endocrinol. Metab. 282:E974-E976, 2002).

GLUT1, together with GLUT3 and GLUT4 which are present in most tissues, has the highest affinity for glucose, and is believed to be responsible for basal glucose uptake (Macheda et al., J. Cell Physiol. 202:654-662, 2005). GLUT1 and GLUT3 are upregulated during transformation, and the level of expression in tumors correlates with decreased survival (Macheda et al., J. Cell Physiol. 202:654-662, 2005). In addition, GLUT3 and GLUT5 are expressed in tumor cells but not the normal cells from which the tumor derives (Macheda et al., J. Cell Physiol. 202:654-662, 2005). As a fructose transporter, GLUT5 is believed to provide the breast cancer cells in which it is overexpressed with an alternative energy source (Zamora-Leon et al., Proc. Natl. Acad. Sci. USA 93:1847-1852, 1996). Of the more recently identified glucose transporters, GLUT12 is widely expressed in embryonic tissues as well as many cancers, but not adult tissues (Rogers et al., Cancer Lett. 193:225-233, 2003). Evidence for the importance of these transporters in cancer comes from studies of their regulation: GLUT1 and GLUT3 are up-regulated in hypoxia; GLUT1 and 4 are negatively regulated by p53; and GLUT12 expression is under the control of estrogens (Macheda et al., J. Cell Physiol. 202:654-662, 2005). Studies with GLUT1-targeting antisense have shown that reducing GLUT1 expression reduces tumor cell growth.

Table 1 lists the SLC2A subfamily members that have been shown to be up-regulated in cancer cells (Macheda et al., J. Cell Physiol. 202:654-662, 2005).

TABLE 1
SLC2A subfamily members up-regulated in cancer cells

| | | DNA SEQ | | | |
|---|---|---|---|---|---|
| Gene | Protein | ID NO: | Substrates | Tissue distribution | Accession |
| SLC2A1 | GLUT1 | 48 | Glucose, | Erythrocytes, brain, | NM_006516 |
| | | | Galactose, Mannose, | blood brain barrier, | |
| | | | glucosamine | blood-tissue barrier | |
| SLC2A3 | GLUT3 | 49 | Glucose, | Brain (neurons), | NM_006931 |
| | | | Galactose, | testis | |
| | | | Mannose, | | |
| | | | Xylose | | |
| SLC2A5 | GLUT5 | 50 | Fructose | Small intestine, | NM_003039 |
| | | | | kidney | |
| SLC2A12 | GLUT12 | 51 | Glucose | Heart, prostate, | NM_145176 |
| | | | | skeletal muscle | |

In a further aspect, the siNA employed in the compositions disclosed herein are capable of suppressing production of one or more transporters in the SLC28 and SLC29A subfamilies of equilibrative nucleoside transporters. Examples of these transporters are listed in Table 2. All cells express proteins involved in uptake of nucleosides. These proteins are sodium-independent equilibrative nucleoside transporters (ENTs), encoded by the SLC29A gene subfamily (Baldwin et al., Eur. J. Physiol. 447:735-743, 2004), and the sodium-dependent concentrative nucleoside transporters (CNTs), encoded by the SLC28A gene subfamily (Gray et al., Eur. J. Physiol. 447:728-734, 2004). These equilibrative nucleoside transporter proteins provide cells with the raw materials for nucleic acid synthesis, as even in cells able to synthesize nucleosides de novo, salvage requires much less energy. The transporters in these two gene families also mediate the uptake of the nucleoside analogue drugs used in chemotherapy. The SLC29A subfamily members have a wider expression profile, whereas the SLC28A subfamily members are expressed at highest levels in epithelial tissues with lower levels at other sites (Gray et al., Eur. J. Physiol. 447:728-734, 2004). Of the four ENTs, the ubiquitously expressed genes encoding ENT1 and ENT2 are the best characterized. These two transporters mediate uptake of both purine and pyrimidine nucleosides containing either ribose or deoxyribose sugar moieties, and in addition, ENT2 also transports nucleobases (Baldwin et al., Eur. J. Physiol. 447:735-743, 2004). As mentioned above, these transporters are able to mediate uptake of analogue drugs (Damaraju, Oncogene 22:7524-7536, 2003), and have shown that loss of expression of SLC29A1 correlates with resistance to nucleoside analogue drugs.

TABLE 2
Nucleoside transporters in cancer cells

| | | DNA | | | |
|---|---|---|---|---|---|
| | | SEQ ID | | | |
| Gene | Protein | NO: | Substrates | Tissue distribution | Accession |
| SLC29A1 | ENT1 | 52 | Purine and | Ubiquitous, plasma | NM_004955 |
| | | | pyrimidine | membrane | |
| | | | nucleosides | (basolateral in | |
| | | | | polarised renal | |
| | | | | epithelial cells) and | |
| | | | | perinuclear | |
| | | | | membranes | |
| SLC29A2 | ENT2 | 53 | Purine and | Ubiquitous, plasma | NM_001532 |
| | | | pyrimidine | membrane | |
| | | | nucleosides | (basolateral in | |
| | | | and nucleobases | polarised renal | |
| | | | | epithelial cells). | |
| | | | | Particularly abundant | |
| | | | | in skeletal muscle | |
| SLC29A3 | ENT3 | 54 | Purine and | Widely distributed, | NM_018344 |
| | | | pyrimidine | possibly intracellular | |
| | | | nucleosides | | |
| | | | and some | | |
| | | | nucleobases | | |
| SLC29A4 | ENT4 | 55 | Adenosine | Widely distributed | NM_153247 |
| SLC28A1 | CNT1 | 56 | Pyrimidines, | Liver, kidney, small | NM_004213 |
| | | | adenosine | intestine | |
| SLC28A2 | CNT2 | 57 | Purines, | Widely distributed | NM_004212 |
| | | | uridine | | |
| SLC28A3 | CNT3 | 58 | Purines and | Widely distributed | NM_022127 |
| | | | pyrimidines | | |

In a further aspect, the siNA employed in the compositions disclosed herein are capable of suppressing production of one or more transporters in the SLC1A, SLC3A, SLC7A, SLC38A and SLC43A subfamilies of amino acid transporters, listed in Table 3. Tumor cells use amino acids for nucleotide, glutathione, amino sugar and protein synthesis, as well as energy production, but consume far more than required to support their growth; leading to negative nitrogen balance in the host (Fuchs and Bode, Sem. Cancer Biol. 15:254-266, 2005). Tumor cells enhance and alter channeling of amino acids to certain pathways, often in concert with the switch to aerobic glycolysis, and are especially dependent on glutamine and arginine for growth and survival (Fuchs and Bode, Sem. Cancer Biol. 15:254-266, 2005). Dedicated amino acid transporters are categorized into several families based on functional attributes. For example, the SLC1A5 (encoding ASCT2), and SLC7A5 (encoding LAT1), obligate exchangers with broad substrate specificity for a number of amino acids, that are upregulated in cancer. Of the high-affinity ASCT2 substrates, glutamine is the most important for cancer cells, whereas LAT1 mediates the import of essential amino acids (Fuchs and Bode, Sem. Cancer Biol. 15:254-266, 2005). LAT1 and ASCT2 both exhibit oncofetal expression patterns: they are overexpressed in cancers but not normal tissue. Inhibition of ASCT2-mediated glutamine uptake, either by inhibition with other ASCT2 substrates or by using an antisense construct, has been shown to slow tumor cell growth (Fuchs et al., Am. J. Physiol. Gastrointest. Liver Physiol. 286:G467-G478, 2004). The SNATs and ATB^{0,+}, from the SLC6A and SLC38A gene families, contribute to cancer cell metabolism by providing exchange partners for glutamine uptake, or by mediating arginine uptake (Gupta, Biophys Biochem Acta 1741:215-223, 2005). A further transporter xCT (encoded by SLC7A11) has also been shown recently to modulate chemosensitivity, by controlling cystine uptake in exchange for glutamate (Huang et al., Cancer Res. 65:7446-7454, 2005). Both LAT1 and xCT form homodimers with 4F2hc, encoded by SLC3A2 (Palacin and Kanai, Eur. J. Physiol. 447:490-494, 2004). This protein is up-regulated on a wide range of tumors and correlates with metastatic potential. In addition to its role in amino acid uptake, contributes to cellular transformation through its signaling function (Palacin and Kanai, Eur. J. Physiol. 447:490-494, 2004).

TABLE 3
Amino acid transporters genes in cancer cells

| | | DNA | | | |
|---|---|---|---|---|---|
| | | SEQ ID | | Tissue | |
| Gene | Protein | NO: | Substrates | distribution | Accession |
| SLC1A4 | ASCT1 | 59 | L-Ala, L-Ser, | Widespread | NM_003038 |
| | | | L-Cys | | |
| SLC1A5 | ASCT2 | 60 | L-Ala, L-Ser, | Lung, skeletal | NM_005628 |
| | | | L-Thr, L-Cys, | muscle, large | |
| | | | L-Gln | intestine, kidney, | |
| | | | | testis, adipose | |
| | | | | tissue | |
| SLC3A2 | 4F2hc, CD98 | 61 | Amino acid | Ubiquitous. In | NM_002394 |
| | | | transport | epithelial cells, | |
| | | | systems L, | basolateral | |
| | | | y + L, xc- and | plasma | |
| | | | asc with light | membrane. | |
| | | | subunits | | |
| | | | SLC7A5-8 | | |
| | | | and | | |
| | | | SLC7A10-11 | | |
| SLC6A14 | ATB^{0,+} | 62 | Arginine | Lung, trachea, | NM_007231 |
| | | | | salivary gland, | |
| | | | | mammary gland, | |
| | | | | stomach, | |
| | | | | pituitary. | |
| SLC7A5 | LAT1 | 63 | Large neutral | Brain, ovary, | NM_003486 |
| | [assoc. | | L-amino | testis, placenta, | |
| | with | | acids, T3, T4, | blood-brain | |
| | 4F2hc] | | L-dopa, BCH | barrier, fetal liver, | |
| | | | (system L) | activated | |
| | | | | lymphocytes, | |
| | | | | tumor cells; lower | |
| | | | | expression in | |
| | | | | many other | |
| | | | | tissues/plasma | |
| | | | | membrane | |
| SLC7A11 | xCT | 64 | Cysteine | Macrophages, | NM_014331 |
| | [assoc. | | (anionic), L- | brain, retinal | |
| | with | | glutamate | pigment cells, | |
| | 4F2hc] | | (system xc-) | liver, kidney/ | |
| | | | | basolateral in | |
| | | | | epithelial cells | |
| SLC43A1 | LAT3 | 65 | Glutamine, | Pancreas and liver | NM_003627 |
| | | | asparagine, | | |
| | | | histidine, | | |
| | | | serine | | |
| SLC38A4 | SNAT3 | 66 | Glutamine, | Brain, liver, | NM_018018 |
| | | | histidine | kidney | |
| SLC38A5 | SNAT5 | 67 | Glutamine, | Stomach, brain, | NM_033518 |
| | | | asparagine, | liver, lung, small | |
| | | | histidine, | intestine, spleen, | |
| | | | serine | colon, kidney | |

In a further aspect, the siNA employed in the compositions disclosed herein are capable of suppressing production of one or more transporters in the SLC5A, SLC19A and SLC23A subfamilies of vitamin transporters (listed in Table 4 below). Vitamins are essential co-factors that cannot be synthesized de novo in cells, and must be absorbed from dietary sources by enterocytes, and subsequently transported to cells. In the case of water-soluble vitamins, uptake is a carrier-mediated process. Biotin, folate, vitamin C (ascorbic acid) and members of the vitamin B complex (thiamine, riboflavin, niacin, pryridoxine) all require transporters to enter cells (Said, Annu. Rev. Physiol. 66:419-446, 2004). Folate transport is carried out by the ubiquitously expressed RFC (encoded by SLC19A1). RFC expression is regulated in some tissues by folate levels (Ganaphthy et al., Eur. J. Physiol. 447:641-646, 2004). RFC also transports methotrexate and other antifolates, and mutations conferring resistance to this class of therapeutic have been characterized (Ganaphthy et al., Eur. J. Physiol. 447:641-646, 2004). Two closely related transporters, ThTr1 and ThTr2, encoded by SLC19A subfamily members, mediate thiamine uptake and are expressed ubiquitously (Ganaphthy et al., Eur. J. Physiol. 447:641-646, 2004). Vitamin C is taken up SVCT1 and SVCT2, encoded by members of the SLC23 subfamily (Takanaga et al., Eur. J. Physiol. 447:677-682, 2004). These proteins provide cells with L-ascorbic acid, which cannot synthesized from glucose in humans due to the lack of the enzyme gulonolactone oxidase (Wilson, Annu. Rev. Nutr. 25:105-125, 2005). SVCT1 is primarily expressed in epithelial tissue and is responsible for the uptake of dietary vitamin C, whereas SVCT2 is expressed ubiquitously (Wilson, Annu. Rev. Nutr. 25:105-125, 2005). The essential nature of these compounds for multiple cellular processes makes inhibiting their uptake an attractive target in cancer cells.

TABLE 4
Vitamin transporter genes expressed in cancer cells

| | | DNA SEQ ID | | | |
|---|---|---|---|---|---|
| Gene | Protein | NO: | Substrates | Tissue distribution | Accession |
| SLC5A6 | SMVT1 | 68 | Biotin, lipoate | Brain, heart, kidney, | NM_021095 |
| | | | and panthothenate | lung and placenta. | |
| | | | | Plasma membranes. | |
| SLC19A1 | RFT, | 69 | N5-methyl- | Ubiquitous/Plasma | NM_003056 |
| | RFC | | tetra | membrane, also in | |
| | | | hydrofolate | mitochondrial | |
| | | | | membrane | |
| SLC19A2 | ThTr1 | 70 | Thiamine | Ubiquitous/Plasma | NM_006996 |
| | | | | membrane | |
| SLC19A3 | ThTr2 | 71 | Thiamine, | Ubiquitous/Plasma | NM_025243 |
| | | | biotin | membrane | |
| SLC23A1 | SVCT1 | 72 | L-Ascorbic | Epithelium of | NM_005847 |
| | | | acid | kidney, intestines, | |
| | | | | liver, ovary, prostate | |
| | | | | and thymus, and B- | |
| | | | | lymphocytes. | |
| SLC23A2 | SVCT2 | 73 | L-Ascorbic | Widespread | NM_005116 |
| | | | acid | including brain | |
| | | | | (neurons), retina, | |
| | | | | placenta, spleen, | |
| | | | | prostate, testis, | |
| | | | | ovary. | |

In a further aspect, the siNA employed in the compositions disclosed herein are capable of suppressing production of one or more mitochondrial transporters (listed in Table 5 below). Mitochondria contain a family of related carrier proteins that mediate transport of metabolites ranging from protons to ATP across the mitochondrial inner membrane. The citrate carrier (CIC/SLC25A1) mediates transport of tricarboxylates, especially citrate, from the mitochondria to the cytosol, and is required for fatty acid and steroid synthesis; the former is elevated in cancer cells (Palmieri, Pflug. Arch. 447:689-709, 2004). The dicarboxylate transporter (DIC/SLC25A10) transports dicarboxylates such as malate or succinate across the mitochondrial inner membrane. Although the primary role of this transporter is to direct Krebs cycle intermediates into the mitochondria, it is also involved in gluconeogenesis including the conversion of amino acids to energy substrates (Palmieri, Pflug. Arch. 447:689-709, 2004).

TABLE 5
Mitochondrial carriers involved in cancer cell metabolism

| | | DNA | | Tissue | |
|---|---|---|---|---|---|
| Gene | Protein | SEQ ID NO: | Substrates | distribution | Accession |
| SLC25A1 | CIC | 74 | Di- and | Liver, kidney, | NM_005984 |
| | | | tricarboxylates | pancreas (also in | |
| | | | | brain, lung, | |
| | | | | heart)/inner | |
| | | | | mitochondrial | |
| | | | | membrane | |
| SLC25A10 | DIC | 75 | Di- and | Liver, kidney, | NM_012140 |
| | | | tricarboxylates | heart, brain, | |
| | | | | lung, pancreas/ | |
| | | | | inner | |
| | | | | mitochondrial | |
| | | | | membrane | |

In a further aspect, the siNA employed in the compositions disclosed herein are capable of suppressing production of one or more transporters in the SLC9A and SLC16A subfamilies involved in controlling intracellular pH (see Table 6 below). In tumor cells, intracellular pH changes during transformation (Cardone et al., Nat. Rev. Cancer 5:786-795, 2005). Both in vitro and in vivo tumor cells have elevated pHi with a lowered extracellular interstitium pH (Cardone et al., Nat. Rev. Cancer 5:786-795, 2005). This reverse pH gradient across the cell membrane increases with tumor progression. Maintenance of this gradient is due to cellular extrusion of protons, which increases with tumor aggressiveness. The NHE1, MCT1 and HCO3—/Cl— exchangers have been shown to be involved in the maintenance of the gradient. Initial transformation steps activate NHE1 thereby increasing pHi, and in transformed cells, an increased affinity of the allosteric proton binding site hyperactivates NHE1, further increasing intracellular pH (pHi) and stimulating aerobic glycolysis. This produces lactate which is extruded by MCT1, further reducing pHi and reversing the cellular pH gradient across the membrane. NHE1 activity also has a fundamental role in controlling glycolysis: stable transfection of ion translocation deficient NHE1 into fibroblasts leads to reduced expression of 6 glycolytic enzymes. NHE1 is activated by low serum, extracelular pH (pHe) and hypoxia, as well as the interaction between hyaluronan and CD44. These factors, together with increased glycolysis, form a feedback loop for tumor progression (Cardone et al., Nat. Rev. Cancer 5:786-795, 2005). Acidification of the microenvironment also stimulates MMP activation. NHE1 small molecule inhibitors have been tested in phase II and III trials. Inhibition of NHE1 is attractive as NHE1 activity is high in tumors and low in normal tissues. Furthermore, AS-ODN targeting NHE1, either alone or in combination with MCT-1, decreased both the growth of A549 cells in vitro and their ability to cause tumors in mice (Zhang et al., Ai Zheng 21:719-723, 2002).

TABLE 6
Genes involved in the regulation of pH in cancer cells

| | | DNA | | | |
|---|---|---|---|---|---|
| Gene | Protein | SEQ ID NO: | Substrates | Tissue distribution | Accession |
| SLC9A1 | NHE1 | 76 | Sodium/proton | Ubiquitous | NM_003047 |
| | | | exchanger | (upregulated in | |
| | | | | cancer) | |
| SLC16A1 | MCT1 | 77 | Lactate and | Ubiquitous | NM_003051 |
| | | | other | (upregulated in | |
| | | | carboxylates | cancer) | |

In a further aspect, the siNA employed in the compositions disclosed herein are capable of suppressing production of one or more transport ATPases. These proteins are sub-classified as P, V, F or ABC ATPases (Pedersen, J. Bioenerg. Biomembr. 37:349-357, 2005). P-type ATPases are characterized by the formation of an aspartyl phosphate intermediate during the reaction cycle and include the well-known Na⁺/K⁺ ATPase, as well as pumps mediating transport of calcium and heavy metals; V-type ATPases are commonly found in the membranes of vacuoles and other organelles, into which they pump protons; F-type ATPases are named for their role in the F₀F₁ ATP synthase; and ABC transporters are named after their ATP-binding cassette, and are responsible for the transport of drugs, xenobiotics, lipids and lipid derivatives against their concentration gradient (Pedersen, J. Bioenerg. Biomembr. 37:349-357, 2005).

P-type ATPases include the plasma membrane Ca²⁺ ATPases PMCA1 (ATP2B1), PMCA2 (ATP2B2), PMCA3 (ATP2B3) and PMCA4 (ATP2B4) (see Table 7 below). Plasma membrane Ca²⁺ ATPases play an important role in regulation of intracellular free Ca²⁺ concentrations required for regulation of Ca²⁺-mediated signaling and other biological processes. Together with Na⁺/Ca²⁺ exchangers, PCMAs are the major plasma membrane transport system responsible for the long-term regulation of the resting intracellular Ca²⁺ concentration (Strehler and Zacharias, Physiol. Rev. 81:21-50, 2001). Studies have shown that aberrant PCMA expression is associated with cancer in several different tissues. Different isoforms of PMCAs are expressed in breast cancer cell lines (Lee et al., Cell Signal. 14:1015-1022, 2002), and PMCA1 is down-regulated in oral cancer (Saito et al., Oncol. Rep. 15:49-55, 2006). The ATP7A and ATP7B transporters mediate the transport of Cu²⁺ and other heavy metals. ATP7B is found predominantly in the liver, kidney and placenta (Komatsu et al., Cancer Res. 60:1312-1316, 2000). ATP7B confers cisplatin resistance when transfected into cells (Komatsu et al., Cancer Res. 60:1312-1316, 2000), and is a marker for cisplatin resistance in solid tumors (Katoh et al., Ann. Thorac. Cardiovasc. Surg. 11:143-145, 2005). ATP7A is expressed at very low (or undetectable) levels in most normal tissues, but is readily found in many common tumor types and is associated with poor prognosis in ovarian cancer patients undergoing platinum-based drug treatments (Samimi et al., Clin. Cancer Res. 9:5853-5859, 2003).

V-type H⁺ ATPase is an ATP-driven enzyme that transforms the energy of ATP hydrolysis to electrochemical potential differences of protons across diverse biological membranes via the primary active transport of H⁺. In turn, the transmembrane electrochemical potential of H⁺ is used to drive a variety of (i) secondary active transport systems via H⁺-dependent symporters and antiporters and (ii) channel-mediated transport systems (Beyenbach and Wieczorak, J. Exp. Biol. 209:577-589, 2006). An intact V-type H⁺ ATPase is required for the normal function of the Golgi complex, endoplasmic reticulum, vacuoles and endocytotic and exocytotic vesicles. Numerous physiological processes depend on the activity of V-ATPases, and V-ATPases are implicated as a contributing factor in multiple diseases, including osteoporosis, deafness, and cancer (Bowman and Bowman, J. Bioenerg. Biomembr. 37:431-435, 2005). Some human tumor cells are characterized by an increased V-type H⁺-ATPase expression and activity (De Milito and Fais, Future Oncol. 1:779-786, 2005) and V-ATPases are known to be involved in multidrug resistance (Sennoune et al., Cell Biochem. Biophys. 40:185-206, 2004).

ABC transporters are a large family comprising 49 members in humans. The first member of this family to be linked with cancer was MDR1 (ABCB1). The product of this gene, P-glycoprotein, uses the energy from ATP hydrolysis to extrude natural product cancer drugs from cells, and its overexpression leads to the phenomenon of multidrug resistance (Borst and Oude-Elferink, Ann. Rev. Biochem. 71:537-592, 2002). It is believed that one way this overexpression occurs is via transcriptional activation controlled by the Y-box binding protein, YBX1 (Bargou et al., Nat. Med. 3:447-450, 1997). Indeed, it has been shown that cells with reduced YBX1 show greater sensitivity to chemotherapeutic compounds (Ohga et al., Cancer Res. 56:4224-4228, 1996). YBX1 (p47) is targeted for specific endoproteolytic cleavage by the 20S proteasome that removes the C-terminal 105 amino-acid residues containing a cytoplasmic retention signal (CRS) (Sorokin et al., EMBO J. 24:1-11, 2005). The resulting N-terminal polypeptide (p37) accumulates in the nuclei of cells exposed to DNA-damaging drugs.

Subsequent to the identification of MDR1, many related genes were identified, including the multidrug-resistance associated (MRP or ABCC) subfamily. Of these, ABCC4 and ABCC5 mediate transport of, among other substrates, nucleoside analog drugs in monophosphate form (Borst and Oude-Elferink, Ann. Rev. Biochem. 71:537-592, 2002), and by contributing to the distribution of these compounds play a role in the relative resistance of cells to these drugs.

TABLE 7
Transport ATPases, ABC transporters and Y-box binding protein involved
in cancer cell physiology

| | | DNA | | Tissue | |
|---|---|---|---|---|---|
| Gene | Protein | SEQ ID NO: | Substrates | distribution | Accession |
| ATP2B1 | PMCA1 | 78 | Ca²⁺ | Ubiquitous in | NM_001001323 |
| | | | | adult tissue | |
| ATP2B2 | PMCA2 | 79 | Ca²⁺ | Nervous | NM_001683 |
| | | | | system, muscles | |
| ATP2B3 | PMCA3 | 80 | Ca²⁺ | Nervous | NM_021949 |
| | | | | system, muscles | |
| ATP2B4 | PMCA4 | 81 | Ca²⁺ | Ubiquitous in | NM_001684 |
| | | | | adult tissue | |
| ATP6V1A | | 82 | H⁺ | Ubiquitous | NM_001690 |
| ATP6V1B2 | | 83 | H⁺ | Ubiquitous | NM_001693 |
| ATP6V1C1 | | 84 | H⁺ | Ubiquitous | NM_001007254 |
| ATP6V1E1 | | 85 | H⁺ | Ubiquitous | NM_001696 |
| ATP6V1F | | 86 | H⁺ | Ubiquitous | NM_004231 |
| ATP7A | | 87 | Heavy | Ubiquitous (not | NM_004047 |
| | | | metals | liver) | |
| ATP7B | | 88 | Heavy | Liver, kidney, | NM_000053 |
| | | | metals | placenta | |
| ABCB1 | MDR1 | 89 | Drugs | Ubiquitous | NM_000927.3 |
| ABCC4 | MRP4 | 90 | Nucleotides | Ubiquitous | NM_005845 |
| ABCC5 | MRP5 | 91 | Nucleotides | Ubiquitous | NM_005688 |
| YBX1 | YBX1 | 92 | | Ubiquitous | NM_004559 |

In an alternative embodiment, the siNA employed in the compositions disclosed herein are targeted against the promoters for the SLC family members, whereby introduction of the genetic construct into a target cell, such as a tumor cell, will lead to transcriptional gene silencing of the genes in the target cell.

In a related aspect, methods are provided for the treatment and/or prevention of a cancer in a patient, comprising administering to the patient a composition disclosed herein. Cancers that may be treated using such methods include, but are not limited to, primary and metastatic tumors and carcinomas of the breast; colon; rectum; lung; oropharynx; hypopharynx; esophagus; stomach; pancreas; liver; gallbladder; bile ducts; small intestine; kidney; bladder; urothelium; cervix; uterus; ovaries; prostate; seminal vesicles; testes; endocrine glands, including thyroid, adrenal and pituitary; skin, including melanomas, sarcomas and Kaposi's sarcoma; tumors of the brain, nerves, eyes and meninges including astrocytomas, gliomas, glioblastomas, retinoblastomas, neuromas, neuroblastomas; and solid tumors arising from hematopoietic malignancies such as leukemias, head and neck cancer, myelomas and lymphomas.

The compositions disclosed herein may include more than one siNA directed against one or more specific cancers. The compositions may be employed alone or in conjunction with known therapeutic methods and compositions currently employed in the treatment of cancer. In a preferred embodiment, the compositions comprising an siNA targeted against a specific target gene that expresses a target protein are employed in conjunction with a known chemotherapeutic directed against the target protein. For example, cancer cells treated with the drugs 2-deoxy-D-glucose and 3-bromopyruvate (both of which target HK-II), and that become drug resistant will still be susceptible to killing using siNA directed against HK-II. The use of siNAs in combination with drugs that target the same cellular protein in a cancer cell may thus have benefits to a patient in terms of reducing the amounts of drugs required and in combating the emergence of drug-resistant cancer cells. In certain embodiments, the disclosed compositions comprise at least one siNA directed against a cancer in combination with at least one known chemotherapeutic agent. Within related embodiments, combination therapeutic regimens for the treatment of disease are provided, such therapeutic regimens comprising the simultaneous and/or sequential administration of one or more siRNA composition(s) in combination with at least one known chemotherapeutic modality.

For example, cancer cells treated with the drug 5-fluorouracil (which targets thymidylate synthase), and that become drug resistant will still be susceptible to killing using siNA directed against thymidylate synthase. The use of siNAs in combination with drugs that target the same cellular protein in a cancer cell may thus have benefits to a patient in terms of reducing the amounts of drugs required and in combating the emergence of drug-resistant cancer cells. Other examples of known cancer therapeutic drugs which may be beneficially employed with the disclosed compositions include, but are not limited to: Xeloda® (capecitabine), Paclitaxel™, and FUDR (fluorouridine), all of which target thymidylate synthase; Fludara® (fludarabine phosphate) and Gemzar® (gemcitabine), both of which target ribonucleotide reductase; methotrexate, which targets both thymidylate synthase and dihydrofolate reductase; and cytarabine, which is incorporated into DNA. In certain embodiments, the compositions disclosed herein comprise at least one siNA directed against a cancer in combination with at least one known chemotherapeutic agent.

In yet other embodiments, the present invention provides systems and methods for the rapid identification of potentially therapeutic small inhibitory RNA (siRNA) molecules, comparison of those siRNA molecules with existing molecules having known in vivo efficacy, and the targeted delivery of those efficacious siRNA into cells associated with a disease phenotype. Such siRNA molecules are capable of mediating RNAi against genes that are active in key pathways involved in disorders, such as, for example, cancers and inflammatory and allergic diseases.

These and other aspects of the present invention will become apparent upon reference to the following detailed description. All references disclosed herein are hereby incorporated by reference in their entirety as if each was incorporated individually.

### BRIEF DESCRIPTION OF THE FIGURES

FIG. 1 shows the linear pathway of enzymes involved in glycolysis together with the sites of ATP utilization and generation

FIG. 2 shows the effect of HK-II binding to the outer membrane of mitochondria via the VDAC receptor.

FIG. 3 depicts proteins involved in transport of molecules across cellular membranes. SLC transporters are represented by circles, whereas pumps are rhomboid in shape with ATP use signified; gene families are numbered without SLC for clarity. Substrates are in bold, co-substrates or exchange partners are in normal font. MCA: monocarboxylic acid; TCA: tricarboxylic acid.

FIG. 4 presents a diagrammatic representation of systems and methods for the identification of siRNA suitable for use in combination therapeutic regimens.

FIG. 5 shows an exemplary RNAi vector of the present invention.

FIG. 6 shows the inhibition of cell growth following transfection of A549 cells with a Stealth™ siRNA duplex targeting the human thymidylate synthetase gene (TYMS).

FIGS. 7A-F show growth inhibition by Stealth™ siRNAs targeting ribonucleotide reductase subunits 1 (RRM1; FIG. 7A-C) and 2 (RRM2; FIG. 7D-F) in A549 (FIGS. 7A and D), SK-MeI-5 (FIGS. 7B and E) and MCF-7 (FIGS. 7C and F) cancer cell lines following transfection with 10 nM of three independent siRNAs. Cells were transfected with specific siRNAs (closed symbols), control siRNA (open triangles) or were untransfected (open circles). Values are average fluorescence units from three replicate wells, representing the amount of DNA per well.

FIG. 8 shows the effect on growth inhibition of A549 cells by 10 nM RRM2B-1 (filled up-triangle) and RRM2B-3 (filled down-triangle) siRNA targeting ribonuclease reductase 2B, measured up to 120 hours post transfection, compared with the effect of a control siRNA (pen triangle) and growth of untransfected cells (open circle).

FIG. 9 shows the dose-dependent effect of RRM1 knockdown on A549 cells that were reverse transfected with increasing concentrations of siRNA. Growth inhibition by RRM1-targeted Stealth™ siRNAs (filled symbols) was measured 96 h post-transfection (filled symbols) and values were normalized to the control siRNA (ctrl-809; open squares) at 0.01 nM.

FIG. 10 shows the dose-dependent effect of RRM2 knockdown on A549 cells that were reverse transfected with increasing concentrations of siRNA. Growth inhibition by RRM2-targeted siRNAs (filled symbols) was measured 96 h post-transfection and values were normalized to the control siRNA (ctrl-809; open squares) at 0.01 nM.

FIG. 11 shows the long-term growth inhibition by RRM1 siRNA in A549 cells reverse transfected with 1 or 10 nM RRM1-3 (filled triangles) or control siRNA (81-c; open triangles). Growth inhibition was measured daily for ten days.

FIGS. 12A and B show the effect of siRNA on ribonucleotide reductase RRM1 mRNA and protein levels. In FIG. 12A, the effect on RRM1 mRNA levels was measured 24 hours after transfecting A549 cells with 1.0, 0.1 or 0.01 nM of RRM1-targeting siRNAs (RRM1-1 (light grey bar); RRM1-2 (dark grey bar); RRM1-3 (black bar)). Reduction in protein levels of A549 cells transfected with 10 nM RRM1-targeting (RRM1-2 and RRM1-3) and control (81-ctrl) siRNA at 48 and 72 hours after transfection measured by Western blotting is shown in FIG. 12B.

FIGS. 13A-D show the effect of the p53 status of wild type HCT-116 (FIGS. 13A and B) and p53 null HCT-116 (FIGS. 13C and D) cells on growth inhibition by RRM1 and RRM2-targeting siRNAs. Cells were reverse transfected with 1.0 nM (closed diamonds) or 10 nM (closed triangles) RRM1-3 (FIGS. 13A and C), RRM2-2 (FIGS. 13B and D) or 10 nM control siRNA (open circles). Growth was measured daily for up to 4 days post transfection.

FIGS. 14A and B show the induction of apoptosis following knockdown of RRM1, with or without treatment with cytarabine. The cell cycle distribution in A549 cells was analyzed following reverse transfection with control or pooled RRM1 siRNAs only (1 or 10 nM final siRNA concentration; FIG. 14A), and transfection with 10 nM pooled RRM1 siRNA together with 10 nM cytarabine (FIG. 14B) over 72 h post-transfection. Values are expressed as the percentage of the gated singlet population, alone and in combination with cytarabine.

FIG. 15 shows the effect of in vitro knockdown on xenograft tumor growth in vivo in CD-1 nude mice injected with A549 cells pre-transfected with 10 nM RRM1-2 (open circles) compared with the effect of control siRNA (open triangles) or untransfected cells (open squares).

FIGS. 16A and B represent the results of two separate experiments showing the effect on A549 xenografted tumors in CD-1 nude mice injected intratumorally with RRM1 siRNA (open circles), PBS (open squares) and a control siRNA (open triangles).

FIG. 16A shows the effect on tumor volume of 50 μg RRM1-targeting siRNA injected 3 times per week for 2 weeks, and FIG. 16B shows the effect on tumor volume of 25 μg siRNA injected 3 times per week for 3 weeks.

FIGS. 17A and B show the growth inhibition in SK-MeI-5 cells (FIG. 17A) and A549 cells (FIG. 17B) reverse transfected with 1.0, 10 or 40 nM of a pool of three siRNAs (closed symbols) targeting SLC1A5 (ASCT2), SLC2Ai (GLUT1), SLC7A5 (LAT1), SLC29A1 (ENT1) or SLC29A2 (ENT2), or a pool of one siRNA against each of the five targets (filled squares), or control siRNAs (control-1; control-2; open symbols). Growth inhibition was measured for 5 days.

FIGS. 18A and B show the reduction in mRNA levels in SK-MeI-5 cells (FIG. 18A) and A549 cells (FIG. 18B) measured 48 h after reverse transfection with 10 nM siRNA (SLC1A5 (ASCT2), SLC2A1 (GLUT1), SLC7A5 (LAT1), SLC29A1 (ENT1) or SLC29A2 (ENT2) or control siRNA (ctrl)).

FIG. 19 shows the growth inhibition by a pool of two YBX1 siRNAs in SK-MeI-5 cells reverse transfected with 0.1, 1.0, 10 or 40 nM YBX1 siRNA (filled squares) or control siRNA (open squares). Growth inhibition was measured after 120 h.

FIG. 20 shows the effect of siRNA on YBX1 protein levels 24 hours after transfecting A549 cells with 10 nM of YBX1-targeting siRNAs (labeled as 94 and 95). Reduction in protein levels was measured by Western blotting.

FIG. 21 shows the effect of siRNA-mediated inhibition of YBX1 gene expression in A549 cells grown in hypoxic versus normoxic conditions. Growth inhibition of A549 cells reverse transfected with 1.0 nM of YBX1-targeting siRNAs labeled 994, 856 and 931, grown for 120 h post-transfection under normoxic conditions (closed symbols) were compared with cells grown for 24 h under normoxic followed by 96 h under hypoxic conditions (open symbols). The percentage fluorescence obtained was normalized against a non-specific control (81ctrl).

FIGS. 22A to D show the effect of co-transfection with p53-targeting siRNA on YBX1 siRNA-mediated growth inhibition in A549, SK-MeI-5, wt HCT-116, p53 null HCT-116 cells. FIG. 22A shows the result of co-transfecting SK-MeI-5 cells with 1 nM p53 907 siRNA and 1 nM of the YBX1-specific siRNA (994; filled circle), compared with transfecting with the YBX1 siRNA alone (filled square) or a control siRNA (81 ctrl; open circle). FIG. 22B shows the result of co-transfecting A549 cells with 5 nM p53 907 siRNA and 5 nM of the YBX1-specific siRNA (open square) compared with transfecting with the YBX1 siRNA alone (filled square) or a control siRNA (open circle), and FIGS. 22C and D show the results of co-transfecting wt HCT-116 and p53 null HCT-116 cells, respectively, with 1 nM p53 907 siRNA and 1 nM of the YBX1-specific siRNA (994; filled circle), compared with transfecting with the YBX1 siRNA alone (filled square) or a control siRNA (open circle).

FIGS. 23A and B show potentiation of 5-FU toxicity in siRNA-transfected SK-Mel-5 cells (FIG. 23A) and A549 cells (FIG. 23B) transfected with 40 nM siRNA targeting SLC1A5, SLC7A5, SLC29A1, SLC29A2 and SLC2A1 (open symbols) and a pool of all five siRNAs (filled triangle), compared with a non-specific control siRNA (filled square).

FIG. 24 shows increased methotrexate toxicity in HCT-15 cells transfected with 40 nM siRNA targeting SLC1A5 (filled diamonds), SLC29A1 (filled triangles), and SLC2A1 (open triangles), compared with a pool of non-specific control siRNAs (filled squares) and mocked transfected cells (filled circles).

FIGS. 25A to C shows the effect of MDR1 knockdown on growth of drug resistance in HCT-15 cells, reverse transfected with 10 nM MDR1-specific siRNA or 10 nM control siRNA and continuously exposed to drug for 72 h. Cells transfected with the MDR1-targeting siRNA (FIG. 25; filled symbols) became more sensitive to the MDR1 substrates doxorubicin (FIG. 25A), paclitaxel (FIG. 25B), and etoposide (FIG. 25C), compared with control siRNA-transfected cells (FIG. 25A-C; open symbols).

FIGS. 26A to D show the effects of TYMS and DHFR knockdown (growth inhibition) on tumor cell growth and sensitivity to 5FU in HCT-116 and A549 cells transfected with 0.1, 1.0 and 10 nM siRNA after 96 h, in either normal or dialyzed fetal calf serum. FIGS. 26A and B shows the effects of knockdown on tumor cell growth and sensitivity to 5FU by TYMS and DHFR siRNA, respectively, in the presence of normal (open bars) or dialyzed fetal calf serum (filled bars) in HCT-116 cells, and FIGS. 26C and D shows the results obtained in A549 cells.

FIGS. 27A to F show the effect of TYMS knockdown on 5FU toxicity in HCT-116 cells transfected with 0.1, 1.0 and 10 nM of three TYMS-specific siRNAs (closed symbols), labeled TYMS-1 (FIGS. 27A and D), TYMS-2 (FIGS. 27B and E) and TYMS-3 (FIGS. 27C and F), or 1.0 and 10 nM control siRNA (809-ctrl; open symbols), and exposed to a serial dilution of 5FU (from 44 μM) continuously for 3 days, starting 24 h after transfection in medium containing either 10% normal FCS (FIGS. 27A to C) or 10% dialyzed FCS (FIGS. 27D to F).

FIGS. 28A to F show the effect of TYMS knockdown on 5FU toxicity in A549 cells transfected with 0.1, 1.0 and 10 nM of three TYMS-specific siRNAs (closed symbols), labeled TYMS-1 (FIGS. 28A and D), TYMS-2 (FIGS. 28B and E) and TYMS-3 (FIGS. 28C and F), or 1.0 and 10 nM control siRNA (81-ctrl; open symbols) and exposed to a serial dilution of 5FU (from at 44 μM) continuously for 3 days, starting 24 h after transfection in medium containing either 10% normal FCS (FIGS. 28A to C) or 10% dialyzed FCS (FIGS. 28D to F).

FIGS. 29A to F show the effect of DHFR knockdown on 5FU toxicity in HCT-116 cells transfected with 0.1, 1.0 and 10 nM of three DHFR-specific siRNAs (closed symbols), labeled DHFR-1 (FIGS. 29A and D), DHFR-2 (FIGS. 29B and E) and DHFR-3 (FIGS. 29C and F), or 1.0 and 10 nM control siRNA (809-ctrl; open symbols) and exposed to a serial dilution of 5FU (from 44 μM) continuously for 3 days, starting 24 h after transfection in medium containing either 10% normal FCS (FIGS. 29A to C) or 10% dialyzed FCS (FIGS. 29D to F).

FIGS. 30A to F show the effect of DHFR knockdown on 5FU toxicity in A549 cells transfected with 0.1, 1.0 and 10 nM of three DHFR-specific siRNAs (closed symbols), labeled DHFR-1 (FIGS. 30A and D), DHFR-2 (FIGS. 30B and E) and DHFR-3 (FIGS. 30C and F), or 1.0 and 10 nM control siRNA (81-ctrl; open symbols) and exposed to a serial dilution of 5FU (from 44 μM) continuously for 3 days, starting 24 h after transfection in medium containing either 10% normal FCS (FIGS. 30A to C) or 10% dialyzed FCS (FIGS. 30D to F).

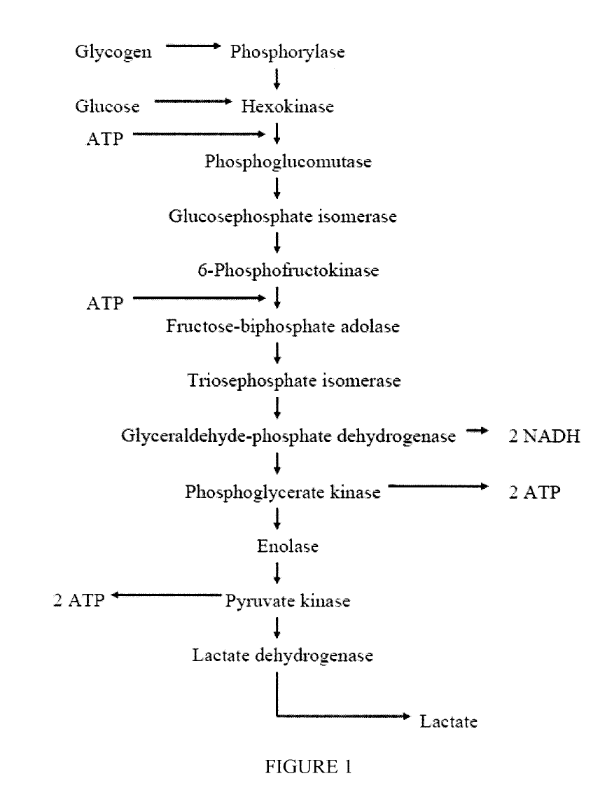

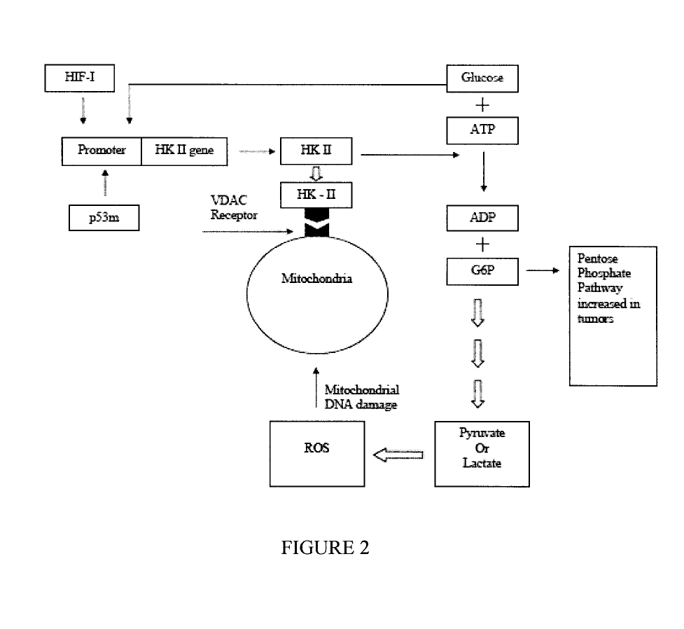

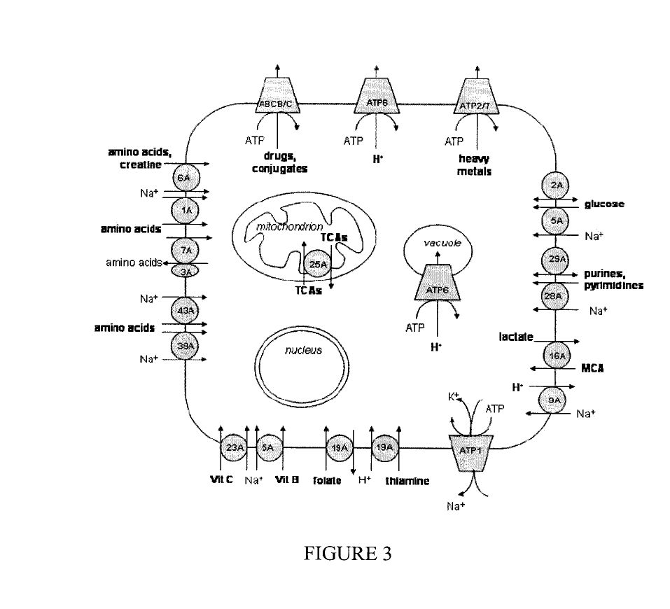

### DETAILED DESCRIPTION OF THE INVENTION

As noted above, the present invention is generally directed to compositions and methods for the treatment of a cancer in a patient. In certain embodiments, the cancer is selected from the group consisting of: primary and metastatic tumors and carcinomas of the breast; colon; rectum; lung; oropharynx; hypopharynx; esophagus; stomach; pancreas; liver; gallbladder; bile ducts; small intestine; kidney; bladder; urothelium; cervix; uterus; ovaries; prostate; seminal vesicles; testes; endocrine glands, including thyroid, adrenal and pituitary; skin, including melanomas, sarcomas and Kaposi's sarcoma; tumors of the brain, nerves, eyes and meninges including astrocytomas, gliomas, glioblastomas, retinoblastomas, neuromas, neuroblastomas, myelomas; head and neck cancers; and solid tumors arising from hematopoietic malignancies such as leukemias and lymphomas.

The compositions disclosed herein comprise at least one unmodified or modified small interfering nucleic acid molecule (siNA) directed against a target gene. In certain embodiments, compositions are provided that comprise a complex including: (a) at least one “naked” (unmodified) or modified small interfering nucleic acid molecule (siNA) directed against a target gene, or a genetic construct that expresses the siNA(s) under the control of a tissue-specific promoter; and (b) a binding agent, such as an antibody, that specifically binds to a target antigen or cell surface molecule which is present on the surface of a target cell of interest. The target antigen recognizes and internalizes certain specific biological molecules, such that, on binding of the siNA-antibody or genetic construct-antibody complex to the target antigen, the complex is internalized into the target cell by endocytosis, the siNA or genetic construct is released from the complex, and the siNA reduces expression of the target gene by means of RNA interference.

As used herein, the term “target gene” refers to a polynucleotide that comprises a region that encodes a polypeptide of interest, and/or a polynucleotide region that regulates replication, transcription, translation or other processes important to expression of the polypeptide of interest.

As used herein, the term “small interfering nucleic acid molecule”, or siNA, refers to any nucleic acid molecule that is capable of modulating the expression of a gene by RNA interference (RNAi), and thus encompasses: short interfering RNA (siRNA); short interfering DNA (siDNA); double-stranded RNA (dsRNA); double-stranded DNA (dsDNA); complementary RNA/DNA hybrids; nucleic acid molecules containing modified (semi-synthetic) base/nucleoside or nucleotide analogues (which may or may not be further modified by conjugation to non-nucleic acid molecules); custom modified primary or precursor microRNA (miRNA); short hairpin RNA (shRNA) molecules; and longer (up to one kb or more) dsRNA or hairpin RNA molecules, so long as these do not activate non-specific interference, for example via interferon. The hairpin region may be short (e.g. 6 nucleotides), long (undefined length), or may include an intron that is efficiently spliced in the targeted cells or tissues. Additionally, multiple tandem repeats in one orientation (for example, three or more short sense repeats) are included under the definition of siRNA, as these can elicit a potent RNAi like response in some systems.

The term siRNA will be used in this disclosure as a prototypical small interfering nucleic acid molecule.

Examples of siNAs that may be effectively employed in the disclosed compositions and methods include the siRNAs provided in SEQ ID NO: 94-4803, 4806-4880, 4889-5003, and 5306-5308 (DNA sequences), and SEQ ID NO: 5112-5298 and 5303-5305 (RNA sequences) which are targeted against the sequences provided in SEQ ID NO: 1-15, and 17-92 as detailed below in Example 3. One of skill in the art will appreciate that, when comparing an RNA sequence to a DNA sequence, an RNA sequence will contain ribonucleotides where the DNA sequence contains deoxyribonucleotides, and further that the RNA sequence will typically contain a uracil at positions where the DNA sequence contains thymidine. Those skilled in the art will also understand that siNAs are double-stranded and that a siNA listed herein will be comprised of the listed sequence together with its complement.

In some embodiments the siNA is generated from an introduced DNA molecule that contains promoter and terminator sequences responsible for transcribing the nucleic acid sequences that comprise the siNA. The introduced DNA may be in the form of a covalently-closed linear or circular plasmid or a PCR product, and these will preferably contain little or no DNA of prokaryotic origin. DNA constructs may also contain a nuclear localization sequence, such as that derived from the SV40 enhancer, to promote nuclear uptake and expression of the construct. In certain embodiments, the siNA is under the control of a promoter that is specific for cancer cells, such as the HIF-1 promoter, the HK-II promoter, or a promoter region containing at least one response element involved in regulating transcription of HK-II, as described, for example, in International Patent Publication no. WO97/04104. Other promoters that may be effectively employed in the genetic constructs include those for c-erbB-2 for the treatment of breast cancer; PSA, osteocalin or clusterin for androgen-dependent and androgen-independent prostate cancers; EIA for melanoma; and survivin for melanoma and other cancers.

For use in conjunction with known cancer therapies, the genetic constructs may employ promoters that are responsive to stimuli currently used in the treatment of cancer. For example, hsp promoters can be activated by photodynamic therapy (Luna et al., Cancer Res. 60:1637-1644, 2000); the mortalin promoter is induced by low doses of ionizing radiation (Sadekova et al., Int. J. Radiat. Biol. 72:653-660, 1997); and the hsp27 promoter is activated by 17b-estradiol and estrogen receptor agonists (Porter et al., J. Mol. Endocrinol. 26:31-42, 2001).

Alternatively, promoters may be of the type activated by RNA polymerase III or RNA polymerase II. Those of the former type include U6, tRNAval, H1, and versions of these promoters modified to achieve higher levels of transcription. Promoters activated by RNA polymerase II may be constitutive (such as the widely used CMV and EF1α promoters), or may be transcribed in a preferred manner in a single cell, cell type, tissue type, or biochemical event. These latter promoters may be chosen for high level or low-level expression.

When a hairpin or custom miRNA is used, a single specific promoter may be employed. When two custom microRNAs with complementary target regions are employed, or when dsRNAs are to be formed from two separate strands, combinations of constitutive and specific, or specific promoters may be employed. In circumstances where reduced, but not eliminated, expression levels are desired, this may be achieved using completely or incompletely homologous antisense siNAs, or using promoters of varying transcriptional activity. Alternatively, siNA may be targeted to regions of mRNA that are either highly affected, or less completely affected, by an siNA, or more than one siNA sequence directed to the target gene, genes or a pathway may be used to achieve stronger interference.

The siNA may be targeted to the 5′ untranslated region, the coding region, or the 3′ untranslated region of the target gene or message. Additionally, regions of the promoter of a target gene, or regions usually upstream of a gene may be targeted for RNAi assisted heterochromatin formation.

An siNA can be unmodified or may be chemically-modified as described, for example, in International Patent Publication nos. WO03/070970, WO03/074654 and WO03/064626; and US Published Patent Applications nos. US2004/0014956, US2004/0192626, US2005/0282188, US2005/0233329, US2005/0020525, US2005/0266422, US2004/0171029, US2004/0171028, US2004/0203024, US2005/0037370, US2004/0171030, US2004/0161777, US2004/0146902, US2005/0119470, US2004/0147470, US2004/0161844, US2004/0171031, US2004/0171032, US2004/0147022, US2004/0147023, US2004/0171033, US2005/0053976, and US2005/0042647, the disclosures of which are hereby incorporated by reference. Where the siNA is a synthetic duplex, this may have symmetrical 3′ overhangs of 2-3 nt, asymmetrical overhangs at one or other end, or may be blunt-ended. Chemical modifications, when present in the duplex, may include nucleic acid residues, or analogs of nucleic acid residues, such as nucleotides with 2′-O-Me- or 2′-fluorine-modified ribose sugars. Such duplexes may be fully (i.e. at every nucleotide) or partially (at selected nucleotide positions) modified, and this modification may be on either the sense strand, the antisense strand, or both strands. Thus, for example, some or all of the nucleotides of an siNA may comprise modified nucleic acid residues, or analogs of nucleic acid residues. The hybridization characteristics of the modified siNA may be similar to or improved compared to the corresponding unmodified siNA. Such modifications can also improve the efficacy and safety of in vivo therapy by changing the stability, lifetime and circulation of the siNAs in the human body.

The siNA may be from 19 to 30 nucleotides in length (for example, 19, 20, 21, 22, 23, 24, 25, 26, 27, 28, 29 or 30 nucleotides) nucleotides in length, and comprises an antisense strand that is complementary to at least a portion of a nucleotide sequence, such as a mRNA sequence corresponding to a target DNA sequence. In certain embodiments, the siNA is 19 to 23 or 25 to 30 nucleotides in length, such as, but not limited to 21, 25 or 27 nucleotides in length. The siNA may also contain a sense strand that comprises the portion of the nucleotide sequence of interest. The sense and antisense strands may be separate, distinct sequences, as in a dsRNA molecule, or may be linked as, for example, in a shRNA molecule.

Those skilled in the art will appreciate that minor changes in the sequence of the siNAs directed against target sequences disclosed herein can yield siNAs that hybridize strongly and specifically to the target nucleic acid. For example, siNAs directed against target sequences that are shifted by one to four nucleotides 5′ or 3′ of the sequences disclosed herein may be effective. It is useful to administer more than one such variant to a target area, or a combination of several different siNAs targeting different regions in and around the desired gene (e.g., exons, introns, promoter, or intergenic regions).

In certain embodiments, the siNA is targeted against a gene or nucleotide sequence that functions in an energy metabolism pathway that is involved in the development and/or progression of a cancer, such as the glycolysis pathway, a gene or nucleotide sequence that is involved in the biosynthesis of nucleotides, or in glycogen metabolism, gene or nucleotide sequence that encodes an electron carrier that works with ATP to provide metabolic energy, or a gene or nucleotide sequence encoding a protein that functions in a transport mechanism that is involved in the development and/or progression of a cancer, such as sugar transport, amino acid transport, water-soluble vitamin transport, transporters controlling intracellular pH, and equilibrative nucleoside transporters.

In specific embodiments, the siNA employed in the compositions and methods disclosed herein is targeted against one or more subsequences listed in Table 8.

TABLE 8

| | | SEQ | |
|---|---|---|---|
| Target mRNA or promoter | Species | ID NO | Gene ID |
| Hypoxia inducible factor 1α (HIF-1α) | Human | 1 | ENST00000337138 |
| Hypoxia inducible factor 1α (HIF-1α) | Mouse | 2 | ENSMUST00000021530 |
| Hypoxia inducible factor 1β (HIF-1β) | Human | 3 | ENST00000322733 |
| Hypoxia inducible factor 1β (HIF-1β) | Mouse | 4 | ENSMUST00000015666 |
| Hexokinase II (HK-II) | Human | 5-7 | ENST00000290573 |
| Hexokinase II (HK-II) | Mouse | 8 | ENSMUST00000000642 |
| At least one promoter element involved | Human | 16 | AF148512 |
| in regulation of HK-II promoter | | | |
| Lactate dehydrogenase 5 (LDH-5) | Human | 9 | ENST00000227157 |
| Lactate dehydrogenase 5 (LDH-5 | Mouse | 10 | ENSMUST00000048209 |
| Phosphofructokinase, liver (PFKL) | Human | 11 | X15573 |
| 6-phosphofructo-2-kinase/fructose-2,6- | Human | 12 | OTTHUMT00000046653 |
| biphosphatase 3 (Pfkfb3) | | | |
| 6-phosphofructo-2-kinase/fructose-2,6- | Mouse | 13, 17 | ENSMUST00000062659 |
| biphosphatase 3 (Pfkfb3) | | | |
| Glucose 6-phosphate dehydrogenase | Human | 14 | OTTHUMT00000061165 |
| (G6PDH) | | | |
| Glucose 6-phosphate dehydrogenase | Mouse | 15 | ENSMUST00000004327 |
| (G6PDH) | | | |
| Phosphate Ribosyl pyrophosphate | Human | 18 | NM_002764 |
| synthase (PRPS1) | | | |
| Amido-phosphoribosyltransferase | Human | 19 | NM_002703 |
| (PPAT) | | | |
| Carbamoyl phosphate synthetase 1 | Human | 20 | ENST00000264705 |
| (CPS1) | | | |
| Carbamoyl phosphate synthetase 2 | Human | 21 | ENST00000264705 |
| (CAD) | | | |
| Ribonucleotide reductase subunit 1 | Human | 22 | ENST00000300738 |
| (RRM1) | | | |
| Thymidylate synthetase (TYMS) | Human | 23 | ENST00000323274 |
| Thymidylate synthetase (TYMS) | Mouse | 24 | ENSMUST00000026846 |
| Dihydrofolate reductase (DHFR) | Human | 25 | ENST00000307796 |
| Adenylate kinase (ATP-AMP | Human | 26 | ENST00000223836 |
| transphosphorylase) AK-1 | | | |
| NAD synthase (nicotinamide adenine | Human | 27 | ENST00000319023 |
| dinucleotide synthase) (NADSYN) | | | |
| FAD synthase (flavin adenine | Human | 28 | ENST00000292180 |
| dinucleotide synthase) (FLAD1) | | | |
| NADH-Q reductase (NADH | Human | 29 | NM_002495 |
| dehydrogenase ubiquinone) | | | |
| Cytochrome reductase (CCR, UQCRC2) | Human | 30 | ENST00000268379 |
| Cytochrome oxidase (COX5a) | Human | 31 | ENSG00000178741 |
| ATP synthase (F0F1-APTase) (ATP5B) | Human | 32 | ENST00000262030 |
| ATP-ADP translocase 1 (SLC25A4; | Human | 33 | NM_001151 |
| ANT1) | | | |
| ATP-ADP translocase 2 (SLC25A5; | Human | 34 | NM_001152.1 |
| ANT2) | | | |
| ATP-ADP translocase 3 (SLC25A6; | Human | 35 | NM_001636.1 |
| ANT3) | | | |
| Fatty Acid Synthase (FASN) | Human | 36 | NM_004104 |
| ATP citrate lyase (ACLY) | Human | 37 | NM_001096 |
| Ribonucleotide reductase subunit 2 | Human | 38 | NM_001034 |
| (RRM2) | | | |
| Ribonucleotide reductase p53 inducible | Human | 39 | NM_015713 |
| (RRM2B) | | | |
| CTP synthase | Human | 40 | NM_001905 |
| Inosine monophosphate dehydrogenase | Human | 41 | NM_000884 |
| 2 (IMPDH2) | | | |
| Deoxycytudune kinase (DCK) | Human | 42 | NM_000788 |
| Thymidine kinase 1 (TK1) | Human | 43 | NM_003258 |
| Thymidine kinase 2 (TK2) | Human | 44 | NM_004614 |
| Deoxyguanosine kinase (DGUOK) | Human | 45 | NM_080916 |
| Uridine monophosphate synthase | Human | 46 | NM_000373 |
| (UMPS) | | | |
| Dihydropyrimidine dehydrogenase | Human | 47 | NM_000110 |
| (DPYD) | | | |
| SLC2A1 (GLUT1) | Human | 48 | NM_006516.1 |
| SLC2A3 (GLUT3) | Human | 49 | NM_006931 |
| SLC2A5 (GLUT5) | Human | 50 | NM_003039.1 |
| SLC2A12 (GLUT12) | Human | 51 | NM_145176.1 |
| SLC29A1 (ENT1) | Human | 52 | NM_004955.1 |
| SLC29A2 (ENT2) | Human | 53 | NM_001532.2 |
| SLC29A3 (ENT3) | Human | 54 | NM_018344 |
| SLC29A4 (ENT4) | Human | 55 | NM_153247 |
| SLC28A1 (CNT1) | Human | 56 | NM_004213 |
| SLC28A2 (CNT2) | Human | 57 | NM_004212 |
| SLC28A3 (CNT3) | Human | 58 | NM_022127 |
| SLC1A4 (ASCT1) | Human | 59 | NM_003038 |
| SLC1A5 (ACST2) | Human | 60 | NM_005628.1 |
| SLC3A2 (4F2hc) | Human | 61 | NM_002394.3 |
| SLC6A14 (ATB^{0,+}) | Human | 62 | NM_007231 |
| SLC7A5 (LAT1) | Human | 63 | NM_003486.4 |
| SLC7A11 (xCT) | Human | 64 | NM_014331.2 |
| SLC43A1 (LAT3) | Human | 65 | NM_003627 |
| SLC38A4 (SNAT3) | Human | 66 | NM_018018 |
| SLC38A5 (SNAT5) | Human | 67 | NM_033518 |
| SLC5A6 (SMVT1) | Human | 68 | NM_021095.1 |
| SLC19A1 (RFT, RFC) | Human | 69 | NM_003056 |
| SLC19A2 (ThTr1) | Human | 70 | NM_006996 |
| SLC19A3 (ThTr2) | Human | 71 | NM_025243 |
| SLC23A1 (SVCT1) | Human | 72 | NM_005847 |
| SLC23A2 (SVCT2) | Human | 73 | NM_005116.5 |
| SLC25A1 (CIC) | Human | 74 | NM_005984 |
| SLC25A10 (DIC) | Human | 75 | NM_012140 |
| SLC9A1 (NHE1) | Human | 76 | NM_003047.2 |
| SLC16A1 (MCT1) | Human | 77 | NM_003051.2 |
| PMCA1 (ATP2B1) | Human | 78 | NM_001001323 |
| PMCA2 (ATP2B2) | Human | 79 | NM_001683 |
| PMCA3 (ATP2B3) | Human | 80 | NM_021949 |
| PMCA4 (ATP2B4b) | Human | 81 | NM_001684 |
| ATP6V1A | Human | 82 | NM_001690 |
| ATP6V1B2 | Human | 83 | NM_001693 |
| ATP6V1C1 | Human | 84 | NM_001007254 |
| ATP6V1E1 | Human | 85 | NM_001696 |
| ATP6V1F | Human | 86 | NM_004231 |
| ATP7A (Menkes protein) | Human | 87 | NM_000052 |
| ATP7B (Wilson Protein) | Human | 88 | NM_000053 |
| ABCB1 (MDR1) | Human | 89 | NM_000927.3 |
| ABCC4 (MRP4) | Human | 90 | NM_005845 |
| ABCC5 (MRP5) | Human | 91 | NM_005688 |
| YBX1 | Human | 92 | NM_004559 |

Methods for selecting suitable regions in a mRNA target are disclosed in the art (see, for example, Vickers et al., J. Biol. Chem. 278:7108-7118, 2003; Elbashir et al., Nature 411:494-498, 2001; Elbashir et al., Genes Dev. 15:188-200, 2001). Preferably, selected target sequences are sensitive to down regulation by low concentrations of siRNA. Guidelines for the design of siNA include those provided in Ambion's Technical Bulletin #506 (available from Ambion Inc., Austin, Tex.), and are described below. The use of low concentrations of siRNA (for example, nanomolar or sub-nanomolar concentrations) and avoidance of sequences that occur in alternative spliced gene products assist in limiting off-target, non-sequence specific, effects. Assessing whether a gene has been down regulated, and the extent of down regulation, can be performed using, for example, real-time PCR, PCR, western blotting, flow cytometry or ELISA methods.

Methods for the preparation of genetic constructs, or expression vectors, comprising, or encoding, siNA targeted against nucleotide sequences of interest are detailed below.

As used herein, the term “binding agent”, refers to a molecule that specifically binds to a target antigen expressed on the surface of a target cells, and includes, but is not limited to, antibodies, including monoclonal antibodies and polyclonal antibodies; antigen-binding fragments thereof, such as F(ab) fragments, F(ab′)₂ fragments, variable domain fragments (Fv), small chain antibody variable domain fragments (scFv), and heavy chain variable domains (V_{HH}); small molecules; hormones; cytokines; ligands; peptides and viruses (either native or modified). Antibodies, and fragments thereof, may be derived from any species, including humans, or may be formed as chimeric proteins which employ sequences from more than one species. The term “binding agent” as used herein thus encompasses humanized antibodies and veneered antibodies.

A binding agent is said to “specifically bind,” to a target antigen if it reacts at a detectable level (within, for example, an ELISA assay) with the target antigen, and does not react detectably with unrelated antigens under similar conditions.

Antibodies, and fragments thereof, may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described, for example, by Kohler and Milstein, Eur. J. Immunol. 6:511-519, 1976, and improvements thereto, via transfection of antibody genes into suitable bacterial or mammalian cell hosts, in order to allow for the production of recombinant antibodies, or by protein synthesis.

In order to minimize any off-target effects, the binding agents employed in the disclosed compositions and methods are cell type-specific. Preferably the binding agent is specific for internalizable cell surface molecules or antigens found on tumor cells. Examples of such molecules include, but are not limited to, the receptors for transferrin, endothelin I and VEGF, including the VEGF-165b isomer. Examples of binding agents that may be usefully employed in the disclosed compositions include: antibodies against transferrin and endothelin available from Abcam Inc. (Cambridge, Mass.), and antibodies against VEGF, available from Delta Biolabs, LLC (Campbell, Calif.), and antigen-binding fragments thereof.

In one embodiment, the compositions disclosed herein comprise a binding agent, such as an antibody, connected to a genetic construct by means of a streptavidin-biotin linkage. As used herein, the term “streptavidin” encompasses both streptavidin and avidin, and derivatives or analogues thereof that are capable of high affinity, multivalent or univalent binding of biotin. Techniques for the preparation of conjugates containing streptavidin-biotin linkages are well known in the art and include, for example, those described in U.S. Pat. Nos. 6,287,792 and 6,217,869, the disclosures of which are hereby incorporated by reference. Biotin may be incorporated into the genetic construct using, for example Biotin-21-dUTP™ (BD Biosciences Clontech, Palo Alto, Calif.), which is a dTTP analog with biotin covalently attached to the pyrimidine ring through a 21-atom spacer arm. The biotin-labeled genetic construct is then linked to the streptavidin-antibody conjugate via biotin-streptavidin binding, using techniques well known to those of skill in the art. Streptavidin-biotin linkers may, alternatively, be employed to link binding agent directly to “naked” siNA.

In a further embodiment, complexes are provided that comprise a binding agent, such as an antibody, and a polynucleotide-binding component, such as a polycation, that is covalently bonded to the antibody through, for example, disulfide bonds. Polycations that may be employed as polynucleotide-binding components include, for example, polylysine, polyarginine, polyornithine, polyethylenimine chitosan and basic proteins, such as histones, avidin and protamines. The polynucleotide-binding component is then attached to a genetic construct by means of electrostatic attraction between the opposite charges present on the genetic construct and the polynucleotide-binding component. The antibody is thus bound to the genetic construct without functionally altering either the genetic construct or the antibody. Both the bond between the antibody and the polynucleotide-binding components and that between the polynucleotide-binding component and the genetic construct are cleaved following internalization of the complex into the target cell. Such complexes may be prepared as described, for example, in U.S. Pat. No. 5,166,320.

Cleavable polymeric linkers which may be effectively employed to attach a genetic construct of the present invention to a binding agent are also described in U.S. Pat. No. 6,627,616.

Alternatively, helicases and other RNA-binding proteins, may be linked to the binding agent, or antibody, and naked siNA is, in turn, linked to the helicase prior to administration. Examples of such helicases and RNA-binding proteins are provided in Sasaki et al., Genomics 82:323-330, 2003, Yan et al., Nature 426:469-474, 2003 and Anderson et al., Mol. Cell. Proteomics, 3:311-326, 2004. In an alternative embodiment, the genetic construct is encapsulated in a liposome or polymer, or attached to a lipid or polymer carrier, which is in turn attached to a binding agent, such as an antibody directed against the target antigen. Encapsulation of the genetic construct within a liposome protects the construct from degradation by endonucleases. Methods for the encapsulation of biologically active molecules, such as nucleic acid molecules and proteins, within liposomes or polymers, and for the preparation of nucleic acid-lipid (lipoplex) and nucleic acid-polymer (polyplex) carrier complexes are well known in the art. See, for example, U.S. Pat. Nos. 6,627,615, 4,241,046, 4,235,871 and 4,394,448; and Liposome Technology: Liposome Preparation and Related Techniques, ed. G. Gregoriadis, CRC Press, 1992. Liposome formulation, development and manufacturing services are available for example, from Gilead Liposome Technology Group (Foster City, Calif.). Lipids for the preparation of liposomes are available, for example from Avanti Polar Lipids, Inc. (Alabaster, Ala.).

The resulting liposome carrier containing the genetic construct of interest is then conjugated to the binding agent, using methods well known in the art, such as those taught in U.S. Pat. Nos. 5,210,040, 4,925,661, 4,806,466 and 4,762,915. Such methods include the use of linkers that fall into four major classes of functionality: conjugation through amide bond formation; disulfide or thioether formation; hydrazone formation; or biotin-streptavidin binding. In a preferred embodiment, the liposome is attached to the binding agent, such as an antibody, by means of a maleimide linker, as described, for example, in U.S. Pat. No. 6,372,250, the disclosure of which is hereby incorporated by reference.

In a preferred embodiment, the liposome employed in the disclosed compositions is a pegylated liposome, wherein the surface of the liposome is conjugated with multiple (up to several thousand) strands of poly(ethylene glycol) (PEG) of approx. 2000 Da. The binding agent is then conjugated to the tips of some of the PEG strands. The diameter of the liposome is preferably within the range of 100 nm to 10 μm. The preparation of such pegylated liposomes and attachment of monoclonal antibodies to the liposomes is performed as described, for example, in Shi and Pardridge, Proc. Natl. Acad. Sci. USA 97:7567-7572, 2000; and Shi et al., Proc. Natl. Acad. Sci. USA 98:12754-12759, 2000. Pegylation of the liposome should increase the stability of the liposome and prevent non-specific attachment of cells, such as macrophages, and proteins to the liposome. The preparation of pegylated liposomes which encapsulate shRNA expression plasmids and are conjugated to monoclonal antibodies, and the use of such compositions in vivo in silencing gene expression in brain cancer is described in Zhang et al., J. Gene Med. 5:1039-1045, 2003.

Alternatively, the siNA or genetic construct is packaged in an adenovirus or adeno-associated virus vector which, upon infection of the target cell, releases its genetic material enabling construct expression. In this embodiment, viral capsid proteins may act as the binding agent and target the siNA or genetic construct to specific cells.

Adenoviruses (AV) and adeno-associated viruses (AAV) do not integrate their genetic material into the host genome and do not require host replication for gene expression. AV and AAV vector delivery systems are thus well suited for rapid and efficient, transient expression of heterologous genes in a host cell. AAV vector delivery systems have previously been shown to be effective in the treatment of cystic fibrosis (Aitken et al., Hum. Gene Ther. 12:1907-1916, 2001). Examples of AAV vector delivery systems which may be effectively employed in the present invention include, but are not limited to, those described in U.S. Pat. No. 6,642,051 and references cited therein. Improvements have been made in the efficiency of targeting adenoviral vectors to specific cells by, for example, coupling adenovirus to DNA-polylysine complexes and by employing strategies that exploit receptor-mediated endocytosis for selective targeting. See, for example, Curiel et al., Hum. Gene Ther. 3:147-154 (1992); and Cristiano and Curiel, Cancer Gene Ther. 3:49-57 (1996).

Alternatively, for situations where stable transfection is desired, viral vectors that insert genetic material into a host cell's genome may be employed. Examples of such vectors include lentiviral, retroviral, plasmid and MLV vectors. The design and use of lentiviral vectors suitable for gene therapy is described, for example, in U.S. Pat. Nos. 6,531,123, 6,207,455 and 6,165,782, the disclosures of which are hereby incorporated by reference. The use of lentivector-delivered RNA interference in silencing gene expression in transgenic mice is described by Rubinson et al. (Nat. Genet. 33:401-406, 2003).

The present invention further provides methods for the treatment of a cancer in a patient by administration of a therapeutically effective amount of a composition disclosed herein.

As used herein, a “patient” refers to any warm-blooded animal, including, but not limited to, a human. Such a patient may be afflicted with disease or may be free of detectable disease. In other words, the methods may be employed for the prevention or treatment of disease. As discussed above, the methods may be employed in conjunction with other known therapies currently employed for the treatment of cancer. For example, the disclosed compositions may be administered before, during or after, radiotherapy, chemotherapy, photodynamic therapy and/or surgery.

In general, the disclosed compositions may be administered by injection (e.g., intradermal, intramuscular, intravenous, intratumoral or subcutaneous), intranasally (e.g., by aspiration), orally, transdermally or epicutaneously (applied topically onto skin). In one embodiment, the compositions are in a form suitable for delivery to the mucosal surfaces of the airways leading to or within the lungs. For example, the composition may be suspended in a liquid formulation for delivery to a patient in an aerosol form or by means of a nebulizer device similar to those currently employed in the treatment of asthma.

For use in therapeutic methods, the disclosed compositions may additionally contain a physiologically acceptable carrier. While any suitable carrier known to those of ordinary skill in the art may be employed in the compositions, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose and magnesium carbonate, may be employed. Biodegradable microspheres (e.g., polylactic galactide) may also be employed as carriers for the compositions. Suitable biodegradable microspheres are disclosed, for example, in U.S. Pat. Nos. 4,897,268 and 5,075,109. Other components, such as buffers, stabilizers, biocides, etc., may be included in the disclosed compositions. The compositions may be provided in single dose or multi-dose containers, such as sealed ampoules and/or vials, and can be stored either frozen or freeze-dried.

The preferred frequency of administration and effective dosage, which will vary from one individual to another and will depend upon the particular disease being treated, may be determined by one skilled in the art, using known techniques. Preferably, siNA is administered at a dose of between 1 and 10 mg/kg. The compositions may be administered in a single dosage or in multiple, divided, dosages.

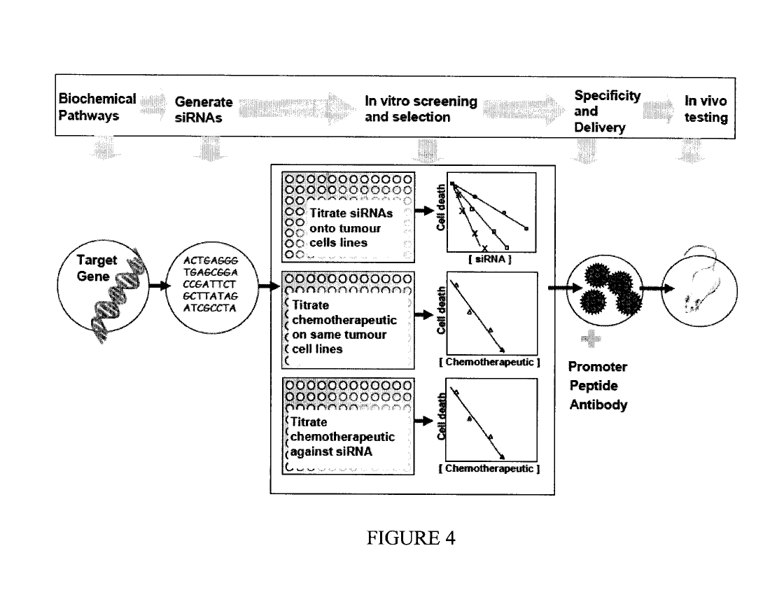

In further embodiments, systems and methods are provided wherein an RNAi therapeutic is compared directly to one or more existing drugs having proven in vivo efficacy for the treatment of a disease state, as illustrated in FIG. 4. In such systems and methods one or more existing therapies serves as a gold standard against which a new RNAi therapy(ies) is compared.

Such systems and methods include a step of comparing the in vitro and in vivo efficacy of one or more existing chemotherapy(ies) to a panel of new siRNA-based therapeutic candidates. Demonstration of the utility of candidate RNAi-based cancer therapeutics can be achieved by comparing the inhibition of gene expression and/or protein function by a known cancer drug in vitro with inhibition of gene expression by a target specific siRNA sequence. For comparative purposes, the siRNA targets are derived from the genes and/or promoters targeted by the known cancer drug. Based on results from such an in vitro comparison, siRNA sequences are selected for further in vivo studies and targeted delivery by cell-specific promoters and/or delivery vehicles. Thus, the systems and methods provided herein are useful to establish the criteria to identify candidate siRNAs that target the same cellular protein and thus have therapeutic potential.

In one example, the disclosed systems and methods are employed for the identification of siRNAs suitable for the treatment of disease(s) susceptible to treatment by modulating the expression of the gene for thymidylate synthetase. In these exemplary systems and methods, a transfectable tumor cell line is titrated with a panel of siRNA candidates designed against the gene for thymidylate synthetase. In parallel, each siRNA candidate is compared to a major current oncology drug, 5-fluorouracil, which specifically targets thymidylate synthetase and therefore serves as a clinical standard against which the efficacy of each siRNA molecule may be assessed.

In an alternative example, systems and methods are provided for the identification of siRNAs suitable for the treatment of disease(s) susceptible to treatment by modulating the expression of the enzyme dihydrofolate reductase. This enzyme is needed to regenerate dihydrofolate from tetrahydrofolate—the reaction that provides energy for thymidylate synthetase as it converts dUMP to dTMP. The drug methotrexate targets the enzyme dihydrofolate reductase and is the current standard of treatment for diseases susceptible to the downregulation of dihydrofolate reductase activity. siRNAs are designed based upon the dihydrofolate reductase gene, titrated in a readily transfectable cell line, and each siRNA's killing activity is compared directly to that of methotrexate.

Thus, systems and methods are provided for identifying novel therapeutics for the treatment of cancers and other diseases using target specific siRNA target sequences, which systems and methods comprise the steps of comparing the inhibition of gene expression with the effect of known cancer drugs on in vitro cells, selecting sequences that inhibit expression, and specifically delivering those siRNAs to the desired target cell. All such systems and methods employ at least one known chemotherapeutic agent that targets specific cellular proteins in a human cancer cell thereby establishing the criteria by which candidate siRNAs having the potential to target the gene encoding the same cellular protein are identified.

In related embodiments, therapeutics, including combination therapeutics, are provided which employ one or more known chemotherapeutics in sequential and/or simultaneous combination with one or more therapeutic siRNA molecules. Such combination therapeutics provide an advantage over stand-alone chemotherapeutics by reducing drug toxicity and/or emergence of drug-resistant cells that no longer respond to the chemotherapeutic alone.

The following Examples are offered by way of illustration and not by way of limitation. Sequences referred to in the Examples below are given in both DNA and RNA nomenclature and represent the sequence of the antisense or guide strands of the siNAs. The final configuration of the strands within the duplexes will consist of a combination of ribonucleotides and deoxyribonucleotides. Further modifications of the phosphodiester backbone and nucleobases, often incorporated into siNAs, have not been explicitly listed, but are well-known in the art.

### EXAMPLE 1

### Preparation of Antibody-Conjugated Liposomes

Preparation of Pegylated Liposomes, Encapsulation of Genetic Constructs and conjugation with monoclonal antibody may be carried out as follows.

1-Palmitoyl-2-oleoyl-sn-glycerol-3-phosphocholine (POPC; Avanti Polar Lipids, Alabaster Ala.; 19.2 μmol), didodecyldimethylammonium bromide (DDAB; Avanti Polar Lipids; 0.2 μmol), distearolyphosphatidylethanolamine ((DSPE)-PEG 2000; Shearwater Polymers, Huntsville, Ala.; 0.6 μmol) and DSPE-PEG 2000-maleimide (30 nmol) are dissolved in chloroform/methanol (2:1, vol:vol) followed by evaporation. The lipids are dispersed in 1 ml 0.05 M Tris-HCl buffer (pH=8.0) and sonicated for 10 min. Supercoiled plasmid DNA is added to the lipids and the liposome/DNA dispersion evaporated to a final concentration of 200 mM at a volume of 100 μl. The dispersion is frozen in ethanol/dry ice for 4-5 min and thawed at 40° C. for 1-2 min. This freeze-thaw cycle is repeated 10 times. The liposome dispersion is then diluted to a lipid concentration of 40 mM, followed by extrusion 10 times each through two stacks of polycarbonate filter membranes. The mean vesicle diameters may be determined using a Microtrac Ultrafine Particle Analyzer (Leeds-Northrup, St. Petersburg, Fla.).

Plasmid attached to the exterior of the liposomes is removed by nuclease digestion as described by Monnard et al. (Biochim. Biophys. Acta 1329:39-50, 1997). For digestion of the unencapsulated DNA, 5 units of pancreatic endonuclease 1 and 5 units of exonuclease II are added in 5 mM MgCl₂ and 0.1 mM DTT to the liposome/DNA mixture after extrusion. After incubation at 37° C. for 1 h, the reaction is stopped by adding 7 mM EDTA.

Monoclonal antibody specific for the target antigen is thiolated using a 40:1 molar excess of 2-iminothiolane (Traut's reagent) as described by Huwyler et al., Proc. Natl. Acad. Sci. USA 93:14164-14169, 1996. Thiolated antibody is then incubated with the liposomes overnight at room temperature, and the resulting immunoliposomes are separated from free monoclonal antibody by, for example, gel filtration chromatography.

### EXAMPLE 2

### Preparation of Peptide-Conjugated Polyplexes

Conjugation of targeting peptides to polycations such as polyethylenimine (PEI), and preparation of peptide-targeted polyplexes may be carried out by the method of Schiffelers et al. (Nucleic Acids Res. 32:1-10, 2004) as follows.

NHS-PEG-VS is obtained from Nektar (San Carlos, Calif.). The targeting RGD peptide with the sequence H-ACDARGDAFCG-OH (SEQ ID NO: 93) is synthesized, oxidized to form the intramolecular disulfide bridge, and purified by reverse-phase HPLC (Auspep Ltd., Parkeville, Australia). The resulting peptide (6 mg) is dissolved in DMSO (60 μl), neutralized with triethylamine (TEA, 2 mol/mol peptide), and coupled to NHS-PEG-VS (21 mg in 40 μl DMSO) for 4 hours at room temperature. The reaction is stopped by adding trifluoroacetic acid (TFA, equimolar to TEA), and the mixture is lyophilized. The intermediate RGD-PEG-VS is purified by dialysis against water, and the compound lyophilized to give a yield of 50-90%. Conjugation is confirmed by mass spectral analysis (matrix-assisted laser desorption ionization).

In the second step of synthesis, various amounts of the purified RGD-PEG-VS intermediate are dissolved in sodium carbonate buffer pH 9.0 (100 μl) and reacted with linear polyethylenimine at room temperature for 16 hours. The reaction is terminated by the addition of an excess of TFA and lyophilized. The product is purified by gel filtration on a Superdex Peptide column in 0.1% TFA, and lyophilized. The degree of conjugation of RGD-PEG to PEI is determined by proton NMR spectrometry on a 400 MHz spectrometer from the ratio of the areas under the peaks corresponding to the —CH₂-protons of PEI (2.8-3.1 ppm) and PEG (3.3-3.6 ppm).

Complexes are formed by mixing equal volumes of solutions of RGD-PEG-PEI and plasmid DNA in HEPES-buffered 5% glucose to give a molar ratio of PEI amine to DNA phosphate of 5:1 to 10:1. The amount of free DNA is quantitated using the Pico Green assay (Invitrogen Corporation, Carlsbad, Calif.).

### EXAMPLE 3

### Design of siRNA Oligonucleotides

Potential target sites in the mRNA are identified based on rational design principles, which include target accessibility and secondary structure prediction. Each of these may affect the reproducibility and degree of knockdown of expression of the mRNA target, and the concentration of siRNA required for therapeutic effect. In addition, the thermodynamic stability of the siRNA duplex (e.g., antisense siRNA binding energy, internal stability profiles, and differential stability of siRNA duplex ends) may be correlated with its ability to produce RNA interference (Schwarz et al., Cell 115:199-208, 2003; Khvorova et al., Cell 115:209-216, 2003). Empirical rules, such as those provided by the Tuschl laboratory (Elbashir et al., Nature 411:494-498, 2001; Elbashir et al., Genes Dev. 15:188-200, 2001) and the Morishita Laboratory (University of Tokyo; Ui-Tei et al., Nucleic Acids Research 32:936-948, 2004) are also used. Software and internet interactive services for siRNA design are available at the following websites: Ambion, Invitrogen, Deqor, Dharmacon, Emboss-2.9.0, Genscript, Cold Spring Harbor Laboratory (Jack Lin), Tuschl Laboratory (MPI), OptiRNA (Cui et al., Computer Methods and Programs in Biomedicine), Qiagen, siDirect and siRNA Design websites. Levenkova et al. describe a software system for design and prioritization of siRNA oligos (Bioinformatics 20:430-432, 2004). The Levenkova system is available on the internet and is downloadable freely for both academic and commercial purposes. The siRNA molecules disclosed herein were based on the Ambion, Invitrogen, Ui-Tei, Deqor, Elbashir and Levenkova recommendations.

The selection of siRNA oligos disclosed in this application was based primarily on uniqueness vs. human sequences (i.e., a single good hit vs. human Unigene, and a big difference in hybridization temperature (Tₘ) against the second best hit) and on GC content (i.e., sequences with % GC in the range of 40-60%).

Optionally, for a more detailed picture on the potential hybridization of the oligos, RNA target accessibility and secondary structure prediction can be carried out using, for example, Sfold software (Ding Y and Lawrence, C. E. (2004) Rational design of siRNAs with Sfold software. In: RNA Interference: from Basic Science to Drug Development. K. Appasani (Ed.), Cambridge University Press; Ding and Lawrence, Nucleic Acids Res. 29:1034-1046, 2001; Nucleic Acids Res. 31:7280-7301, 2003). Sfold is available on the internet. RNA secondary structure determination is also described in Current Protocols in Nucleic Acid Chemistry, Beaucage et al., ed., 2000, at 11.2.1-11.2.10.

The targeted region is selected from a cDNA sequence, such as the cDNA sequence for lactate dehydrogenase 5 (SEQ ID NO: 9). Potential target sequences and positions are typically identified by searching for specific 23 nucleotide (nt) motifs (“Tuschl patterns” such as AA(N19)TT, where N is any nucleotide, and AA is referred to herein as the “target motif leader”, NA(N21), or BA(N21), where B=C, G, U; Elbashir S M et al., Methods 26:199-213, 2002) in the cDNA sequence, starting at about 50-100 nt downstream of the start codon. The nt 22 and nt 23 need not be considered in searching for Tuschl patterns, since they are not involved in the base pairing between the mRNA target and the antisense siRNA strand. “Sense siRNA” is used herein to mean a target sequence without the NN leader. For example, the sequence of the sense siRNA corresponds to (N19)TT of the Tuschl pattern AA(N19)TT (positions 3-23 of the 23 nt motif).

The siRNAs may be designed with symmetric 3′ overhangs in order to form a symmetric duplex (Elbashir et al., EMBO J. 20:6877-6888, 2001). For both sense and antisense siRNAs, either dTdT or UU are used as the 3′ overhang. Thus for siRNAs with an AA target motif leader, the AA base pairs with the dTdT or UU overhang of the antisense siRNA. For BA leaders, the A pairs with the first dT or U of the overhang. It is known however, that the overhang of the sense sequence can be modified without affecting targeted mRNA recognition.

The antisense siRNA is synthesized as the complement to position 1-21 of the 23 nt motif. The 3′ most nucleotide can be varied, but the nucleotide at position 2 of the 23 nt motif is selected to be complementary to the targeted sequence. These methods are well known in the art. Where it is desired to efficiently express RNAs from pol III promoters, the first transcribed nt should be a purine. For example, the siRNA may be selected corresponding to the target motif NAR(N17) YNN, where R is (A,G) and Y is (C,U). The target sequence motifs are selected to have about 30-70% GC content, preferably 40-60% GC content. As used herein, the “% GC content” is calculated as: [the number of G or C nucleotides in the target sequence/21 for an AA target motif leader]×100, [the number of G or C nucleotides in the target sequence/20 for a BA target motif leader]×100, and [the number of G or C nucleotides in the target sequence/19 for an NB target motif leader]×100.

Following selection of siRNA duplexes from the target sequence, the thermodynamic properties of the sequences are determined, e.g., using the Sfold software referred to above. As used herein, “DSSE” refers to the differential stability of the siRNA duplex ends, i.e., the average difference between 5′ antisense and 5′ sense free energy values for the four nucleotide base pairs at the ends of the duplex. It has been shown that the 5′ antisense region is less stable than the 5′ sense terminus in functional siRNA duplexes and vice versa for nonfunctional siRNA duplexes (Khvorova et al., Cell 115:209-216, 2003). It is known that the siRNA duplex can be functionally asymmetric, in the sense that one of the two strands preferentially triggers RNAi (Schwartz et al., Cell 155:199-208, 2003).

As used herein, “AIS” refers to the average internal stability of the duplex at positions 9-14 from the 5′ end of the antisense strand. Comparisons between functional and nonfunctional siRNA duplexes indicate that the functional siRNA has lower internal stability in this region. It is proposed that flexibility in this region may be important for target cleavage (the mRNA is cleaved between position 9 and 10) and/or release of cleaved products from RISC to regenerate RISC (Khvorova et al., Cell 115:209-216, 2003).

The siRNA sequences and their thermodynamic properties are further selected according to the following criteria: (a) 40%≦GC content≦60%; (b) antisense siRNA binding energy ≦−15 kcal/mol; and (c) exclusion of target sequence with at least one of AAAA, CCCC, GGGG or UUUU. For siRNAs with NN dinucleotide leaders, two additional criteria are used: (d) DSSE>0 kcal/mol (Zamore asymmetry rule); and (e) AIS>−8.6 kcal/mol (cleavage site instability rule). This is the midpoint between the minimum of −3.6 and maximum of −13.6 (Khvorova et al., Cell 115:209-216, 2003).

To increase the likelihood that only one gene will be targeted for degradation, the selected siRNA sequences are further checked for uniqueness against human and murine gene libraries (e.g., TIGR GI, ENSEMBL human genome), using Blast algorithms. Also, to increase the likelihood that the selected sequences will be active, sequences directed against targets having SNPs in the base pairing regions are excluded.

The exemplary siRNAs provided in SEQ ID NO: 94-163, 234-1883, 2004-4803, 4806-4868, 4872-4880, 4889-5003, 5112-5298, and 5303-5308 correspond to the target sequences provided in SEQ ID NO: 1-15, and 17-92, as shown in Table 9 below. SEQ ID NO: 94-163, 234-1883, 2004-4803, 4806-4868, 4872-4880, 4889-5003, 5112-5298, and 5303-5308 represent the antisense of the target sequences and are listed as both DNA and RNA versions to include all nucleic acid possibilities targeting those sequences (for example RNA, DNA, mixmer, modified bases and modified backbones).

TABLE 9

| | Alternative | Target | Antisense | Antisense |
|---|---|---|---|---|
| | nomenclature or | SEQ ID | DNA | RNA |
| Gene nomenclature | abbreviation | NO: | SEQ ID NO: | SEQ ID NO: |
| HIF-1α | HIF-1α | 1 | 94-103 and | |
| | | | 234-473 | |
| HIF-1β | ARNT | 3 | 104-113, | 5112-5114 |
| | | | 474-823 and | |
| | | | 4806-4808 | |
| Hexokinase II | HK-II | 5-7 | 114-133, | 5118-5120 |
| | | | 824-1093 | |
| | | | and 4812-4814 | |
| LDH-A | Lactate | 9 | 134-143, | 5121-5123 |
| | dehydrogenase 5; | | 1094-1343 | |
| | LDH-5 | | and 4815-4817 | |
| Pfkfb3 | PFKFB3 | 12, 17 | 144-153, | |
| | | | 1344-1533, | |
| | | | 1764-1773 | |
| | | | and 2004-2193 | |
| G6PDH | H6PDH | 14 | 154-163, | 5115-5117 |
| | | | 1534-1763 | |
| | | | and 4809-4811 | |
| Phosphate ribosyl | PRPS1 | 18 | 4821-4823 | 5127-5129 |
| pyrophosphate | | | | |
| synthase | | | | |
| Carbamoyl | CPS1 | 20 | 1774-1783, | 5130-5132 |
| phosphate synthetase 1 | | | 2194-2333, | |
| | | | 4824-4826 | |
| Carbamoyl | CAD | 21 | 1784-1793, | 5133-5135 |
| phosphate synthetase 2 | | | 2334-2513, | |
| | | | 4827-4829 | |
| Ribonucleotide | RRM1 | 22 | 1794-1803, | 5136-5138 |
| reductase subunit 1 | | | 2514-2833, | |
| | | | 4830-4832 | |
| Thymidylate | TYMS | 23 | 1804-1813, | 5139-5141 |
| synthetase | | | 2834-3023, | |
| | | | 4833-4835 | |
| Dihydrofolate | DHFR | 25 | 1814-1823, | 5142-5144 |
| reductase | | | 3024-3333, | |
| | | | 4836-4838 | |
| Adenylate kinase | AK1 | 26 | 1824-1833, | 5145-5147 |
| | | | 3334-3503, | |
| | | | 4839-4841 | |
| NAD synthase | NADSYN | 27 | 1834-1843, | 5148-5150 |
| | | | 3504-3733, | |
| | | | 4842-4844 | |
| FAD synthase | FLAD1 | 28 | 1844-1853, | 5151-5153 |
| | | | 3734-4053, | |
| | | | 4845-4847 | |
| Cytochrome oxidase | COX5A | 31 | 1864-1873, | 5154-5156 |
| | | | 4364-4533, | |
| | | | 4848-4850 | |
| ATP synthase | ATP5B | 32 | 1874-1883, | 5157-5159 |
| (F0F1-APTase) | | | 4534-4803, | |
| | | | 4851-4853 | |
| Fatty Acid Synthase | FASN | 36 | 4854-4856 | 5160-5162 |
| ATP Citrate Lyase | ACLY | 37 | 4857-4859 | 5163-5165 |
| ATP-ADP | SLC25A5 | 34 | 4860-4862 | 5166-5168 |
| translocase; ANT2 | | | | |
| ATP-ADP | SLC25A6 | 35 | 4863-4865 | 5169-5171 |
| translocase; ANT3 | | | | |
| Cytochrome | UQCRC2 | 30 | 1854-1863, | 5172-5174 |
| reductase | | | 4054-4363, | |
| | | | 4866-4868 | |
| Ribonucleotide | RRM2 | 38 | 5306-5308 | 5303-5305 |
| reductase subunit 2 | | | | |
| Ribonucleotide | RRM2B | 39 | 4872-4874 | 5175-5177 |
| reductase subunit 2B | | | | |
| CTP synthase | CTPS | 40 | 4875-4877 | 5178-5180 |
| Inosine | IMPDH2 | 41 | 4878-4880 | 5181-5183 |
| monophosphate | | | | |
| dehydrogenase 2 | | | | |
| Deoxycytidine | DCK | 42 | 4986-4988 | 5281-5283 |
| kinase | | | | |
| Thymidine kinase 1 | TK1 | 43 | 4989-4991 | 5284-5286 |
| Thymidine kinase 2 | TK2 | 44 | 4992-4994 | 5287-5289 |
| Deoxyguanosine | DGUOK | 45 | 4995-4997 | 5290-5292 |
| kinase | | | | |
| Uridine | UMPS | 46 | 4998-5000 | 5293-5295 |
| monophosphate | | | | |
| synthase | | | | |
| Dihydropyrimidine | DPYD | 47 | 5001-5003 | 5296-5298 |
| dehydrogenase | | | | |
| ASCT2 | SLC1A5 | 60 | 4889-4891 | 5184-5186 |
| GLUT1 | SLC2A1 | 48 | 4892-4894 | 5187-5189 |
| GLUT5 | SLC2A5 | 50 | 4895-4897 | 5190-5192 |
| GLUT12 | SLC2A12 | 51 | 4898-4900 | 5193-5195 |
| LAT1 | SLC7A5 | 63 | 4901-4903 | 5196-5198 |
| xCT | SLC7A11 | 64 | 4904-4906 | 5199-5201 |
| 4F2hc | SLC3A2 | 61 | 4907-4909 | 5202-5204 |
| SMVT1 | SLC5A6 | 68 | 4910-4912 | 5205-5207 |
| ThTr1 | SLC19A2 | 70 | 4913-4915 | 5208-5210 |
| SVCT2 | SLC23A2 | 73 | 4916-4918 | 5211-5213 |
| NHE1 | SLC9A1 | 76 | 4919-4921 | 5214-5216 |
| MCT1 | SLC16A1 | 77 | 4922-4924 | 5217-5219 |
| CIC | SLC25A1 | 74 | 4925-4927 | 5220-5222 |
| DIC | SLC25A10 | 75 | 4928-4930 | 5223-5225 |
| ENT1 | SLC29A1 | 52 | 4931-4933 | 5226-5228 |
| ENT2 | SLC29A2 | 53 | 4934-4936 | 5229-5231 |
| ATP2B1 | PMCA1 | 78 | 4937-4939 | 5232-5234 |
| ATP2B2 | PMCA2 | 79 | 4940-4942 | 5235-5237 |
| ATP2B3 | PMCA3 | 80 | 4943-4945 | 5238-5240 |
| ATP2B4 | PMCA4 | 81 | 4946-4948 | 5241-5243 |
| ATP6V1A | ATPase, H+ | 82 | 4949-4951 | 5244-5246 |
| | transporting, | | | |
| | lysosomal 70 kDa, | | | |
| | V1 subunit A | | | |
| ATP6V1B2 | ATPase, H+ | 83 | 4952-4954 | 5247-5249 |
| | transporting, | | | |
| | lysosomal 56/58 kDa, | | | |
| | V1 subunit B2 | | | |
| ATP6V1C1 | ATPase, H+ | 84 | 4955-4957 | 5250-5252 |
| | transporting, | | | |
| | lysosomal 42 kDa, | | | |
| | V1 subunit C1 | | | |
| ATP6V1E1 | ATPase, H+ | 85 | 4958-4960 | 5253-5255 |
| | transporting, | | | |
| | lysosomal 31 kDa, | | | |
| | V1 subunit E1 | | | |
| ATP6V1F | ATPase, H+ | 86 | 4961-4963 | 5256-5258 |
| | transporting, | | | |
| | lysosomal 14 kDa, | | | |
| | V1 subunit F | | | |
| ATP7A | ATPase, Cu++ | 87 | 4964-4966 | 5259-5261 |
| | transporting, alpha | | | |
| | polypeptide | | | |
| ATP7B | ATPase, Cu++ | 88 | 4967-4969 | 5262-5264 |
| | transporting, beta | | | |
| | polypeptide | | | |
| ABCC4 | ABC transporter; | 89 | 4970-4972 | 5265-5267 |
| | MRP4 | | | |
| ABCC5 | ABC transporter; | 90 | 4973-4975 | 5268-5270 |
| | MRP5 | | | |
| ABCB1 | ABC transporter; | 91 | 4976-4980 | 5271-5275 |
| | MDR1 | | | |
| YBX1 | Y Box-binding | 92 | 4981-4985 | 5276-5280 |
| | protein 1; YB-1 | | | |

### EXAMPLE 4

### Synthesis and Testing of siRNA Duplexes

siRNA may be prepared by various methods, for example by chemical synthesis, or from suitable templates using in vitro transcription, siRNA expression vectors or PCR generated siRNA expression cassettes. Preferably, chemical synthesis is used.

Methods for chemical synthesis of RNA are well known in the art and are described, for example, in Usman et al., J. Am. Chem. Soc. 109:7845, 1987; Scaringe et al., Nucleic Acids Res. 18:5433, 1990; Wincott et al., Nucleic Acids Res. 23:2677-2684, 1995; and Wincott et al., Methods Mol. Biol. 74:59, 1997. Twenty-one nucleotide siRNAs may be synthesized, for example, using protected ribonucleoside phosphoramidites and a DNA/RNA synthesizer, and are commercially available from a number of suppliers, such as Proligo (Hamburg, Germany), Dharmacon Research (Lafayette, Colo.), Perbio Science (Rockford, Ill.), Glen Research (Sterling, Va.), ChemGenes (Ashland, Mass.), and Ambion Inc. (Austin, Tex.). The siRNA strands can then be deprotected, annealed and purified before use, if necessary. Annealing can be carried out, for example, by incubating single-stranded 21-nt RNAs in 100 mM potassium acetate, 30 mM HEPES-KOH at pH 7.4, 2 mM Mg acetate, 1 min at 90° C., then 1 hr at 37° C. The solution is then stored frozen at −20° C. Useful protocols can be found in Elbashir et al., Methods 26:199-213, 2002.

### EXAMPLE 5

### RNAi Expression Vectors

Expression vectors for generating siRNA fragments against selected gene targets are constructed by ligating annealed, chemically synthesized, oligonucleotide pairs into the appropriate vector (for example pSilencer (Ambion) or pSiren (BD Bioscience)), or by PCR amplification of cDNA corresponding to siRNA sequences. For expression from vectors, siRNA sequences should start with 5′G residues. Symmetric 3′ overhangs and appropriate restriction sequences may be added during amplification or within the synthesized oligonucleotides. The amplified sequences are subcloned into, for example, pcDNA3 vectors (Invitrogen, Carlsbad, Calif.).

Tumor cell promoters, such as the HK-II promoter (SEQ ID NO: 16), are cloned into expression vectors containing a fluorescent reporter gene, such as EGFP, and tested in human and murine cancer cell lines (American Type Culture Collection (ATCC), Manassas, Va.), for their ability to confer tumor cell-specific expression. Based on these experiments, appropriate promoters are selected and subcloned into specific RNAi vectors.

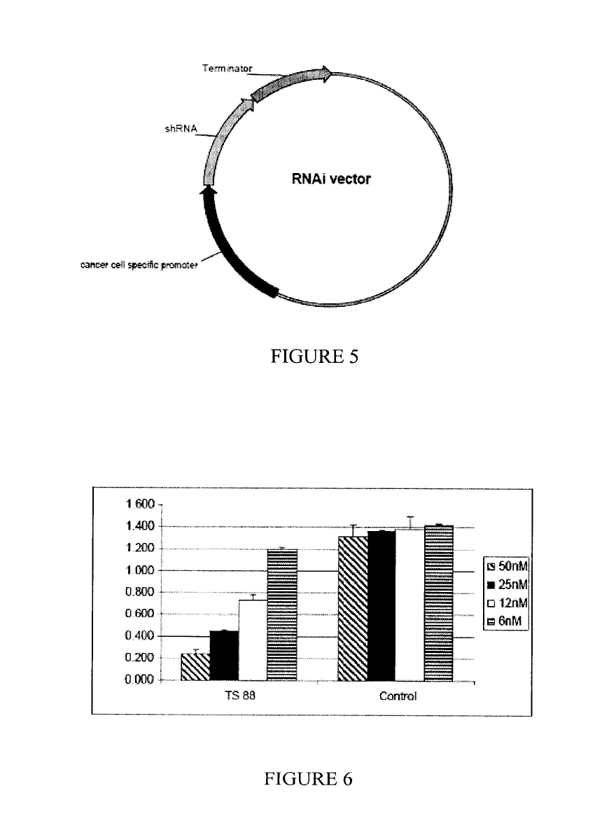

An exemplary RNAi vector is shown in FIG. 5. The vector can be constructed based on commercially available vectors such as pSilencer from Ambion and comparable vectors from other suppliers. Alternatively, covalently-closed linear constructs, containing only the shRNA expression cassette, can be used. These constructs can be generated by PCR or restriction digestion followed by ligation of short hairpin oligos to yield endonuclease-resistant covalently-closed molecules. In a further embodiment, these constructs may contain nuclear localization sequences to promote nuclear uptake and expression of the construct. Such sequences may be either encoded in the DNA (Dean, Exp. Cell. Res. 230:293-302, 1997) or covalently linked peptide moieties (Zanta et al., Proc. Nat. Acad. Sci. USA 96:91-96, 1999).

### EXAMPLE 6

### RNAi-Directed Transcriptional Silencing

For long-term suppression of target gene expression, it would be advantageous to silence transcription by producing double-stranded RNAi in the nucleus that is capable of triggering transcriptional gene silencing of target gene expression. This may be done by introducing RNAi constructs that are expressed in the nucleus, and that contain promoter sequences directed against the target gene promoter or the promoter of a transcription factor that activates the target gene promoter. RNAi-dependent chromatin silencing has been demonstrated in both fission yeast and plants (reviewed by Matzke and Matzke, Science 301:1060-1061, 2003). In plants, the synthesis of double-stranded RNA containing promoter sequences triggers transcriptional gene silencing and methylation of the target promoter (Mette et al., EMBO J. 19:5194-5201, 2000).

Examples of short hairpin RNA sequences are given in SEQ ID NO: 164 to 173 for inhibition of HIF-1α; SEQ ID NO: 174 to 183 for inhibition of HIF-1β; SEQ ID NO: 184 to 203 for inhibition of hexokinase II; SEQ ID NO: 204 to 213 for inhibition of lactate dehydrogenase 5; SEQ ID NO: 214 to 223 and 1884 to 1893 for inhibition of pfkfb3; SEQ ID NO: 224 to 233 for inhibition of G6PDH; SEQ ID NO: 1894 to 1903 for inhibition of CPS1; SEQ ID NO: 1904 to 1913 for inhibition of CAD; SEQ ID NO: 1914 to 1923 for inhibition of ribonucleotide reductase subunit 1; SEQ ID NO: 1924 to 1933 for inhibition of thymidylate synthetase; SEQ ID NO: 1934 to 1943 for inhibition of dihydrofolate reductase; SEQ ID NO: 1944 to 1953 for inhibition of adenylate kinase; SEQ ID NO: 1954 to 1963 for inhibition of NAD synthase; SEQ ID NO: 1964 to 1973 for inhibition of FAD synthase; SEQ ID NO: 1974 to 1983 for inhibition of cytochrome reductase; SEQ ID NO: 1984 to 1993 for inhibition of cytochrome oxidase; and SEQ ID NO: 1994 to 2003 for inhibition of ATP synthase.

Expression cassettes are designed to express siRNAs in the nucleus under the control of a human U6 snRNA promoter or a tissue specific promoter (see, for example, Miyagishi and Taira, Nature Biotechnol. 20:497-500, 2002; Paul et al., ibid, 505-508). The cassette also contains U6 termination sequences. The desired target gene promoter sequences or transcription factor sequences are subcloned into the cassette, e.g., a pU6 plasmid, or a linear derivative of such a plasmid. To promote nuclear uptake, these constructs can be engineered to include nuclear localization sequences. Various strategies may be tested, including the production of short hairpin siRNAs containing one or more inverted DNA repeats and/or tandem DNA repeats of promoter-containing sequences, and synthesis of separate sense and antisense promoter RNAs in a single construct with two different promoters.

Guidelines for constructing hairpin siRNA expression cassettes may be found, for example, in the Ambion Technical Bulletin #506 (Ambion Inc., Austin, Tex.).

Chromatin silencing in cells transfected with nuclear-targeted siRNA vectors is assessed by methods to detect gene-specific mRNA or protein expression such as quantitative PCR, Northern blotting, ELISA, flow cytometry and western blotting. Alternatively, this may be assessed by methylation analysis of the promoter region using methylation-specific restriction enzyme analysis, methylation-specific PCR, or bisulfite treatment of genomic DNA fragments followed by cloning and sequencing.

### EXAMPLE 7

### siRNA Mediated Silencing of Thymidylate Synthase

siRNA mediated silencing in Human A549 cells A549 cells (ATCC No. CCL-185), a human lung cancer cell line, were cultured in RPMI 1640 medium with 5% v/v dialyzed fetal calf serum. Cells were seeded at a density of 2×10⁴ cells/100 μl in 96-well plates. Stealth™ siRNA duplexes targeting the thymidylate synthase (SEQ ID NO: 23) gene were transfected at concentrations of 6, 12, and 50 nM into the A549 cells using Lipofectamine 2000 transfection reagent (Invitrogen). After 72 h, cell viability was assessed by measuring cell proliferation using a standard MTT assay (Mosman, J. Immunol. Methods 65:55-63, 1983).

Transfecting cells with the thymidylate synthetase-targeting duplex (SEQ ID NO: 4883 (DNA), and SEQ ID NO: 5139 (RNA)) lead to a dose-dependent inhibition of cell growth (FIG. 6). Using a control duplex (SEQ ID NO: 4804 (DNA), and SEQ ID NO: 5301 (RNA)) against an irrelevant target, no effect was observed.

### EXAMPLE 8

### siRNA-Mediated Inhibition of Ribonuclease Reductase

### In Vitro Growth Inhibition

A549 cells (human lung cancer cell line; ATCC No. CCL-185), SK-MeI-5 cells (human melanoma cell line; ATCC No. HTB-70), MCF-7 cells (human adenocarcinoma cell line; ATCC No. HTB-22), HCT-116 cells (colorectal carcinoma; ATCC CCL-247) and HCT-116 p53 null cells (derived from ATCC No. CCL-247; Bunz et al., Science 282:1497-1501, 1998) were cultured in RPMI 1640 medium with 10% v/v heat-inactivated fetal calf serum (FCS). Cells were seeded at a density of 5×10⁴ cells/cm² in multi-well tissue culture plates. Sequences of Stealth™ siRNA duplexes (Invitrogen) targeting human ribonucleotide reductase subunit 1 (SEQ ID NO: 22) and subunit 2 (SEQ ID NO; 38) are given in SEQ ID NO: 4830-4832 (DNA), and SEQ ID NO: 5136-5138 (RNA) for RRM1, and SEQ ID NO: 5306-5308 (DNA), and SEQ ID NO: 5303-5305 (RNA) for RRM2. siRNAs were transfected at different concentrations into cells using Lipofectamine™ RNAiMAX transfection reagent (Invitrogen). Non-specific siRNA (SEQ ID NO: 4804 and 4805 (DNA), and SEQ ID NO: 5301 and 5302 (RNA)) were used as controls. The cellular DNA content was measured using a SYBR Green I-based fluorometric assay at 24, 48, 72 and 96 hours after transfection (120 hours for the MCF-7 cells), as follows. At the indicated time points following reverse transfection, cells were harvested and frozen at −80° C. overnight. Cells were thawed and cell lysis buffer (phosphate-buffered saline (PBS) containing 1% Triton X-100, pH 7) and SYBR Green I (1:10⁵ V/V; Invitrogen) was added to the wells and incubated overnight in the dark at 4° C.

The following day, cell lysates were mixed thoroughly, and DNA fluorescence was measured with a Wallac Victor² plate reader (Turku, Finland) set at an excitation frequency of 485 nm and measuring emission at 535 nm.

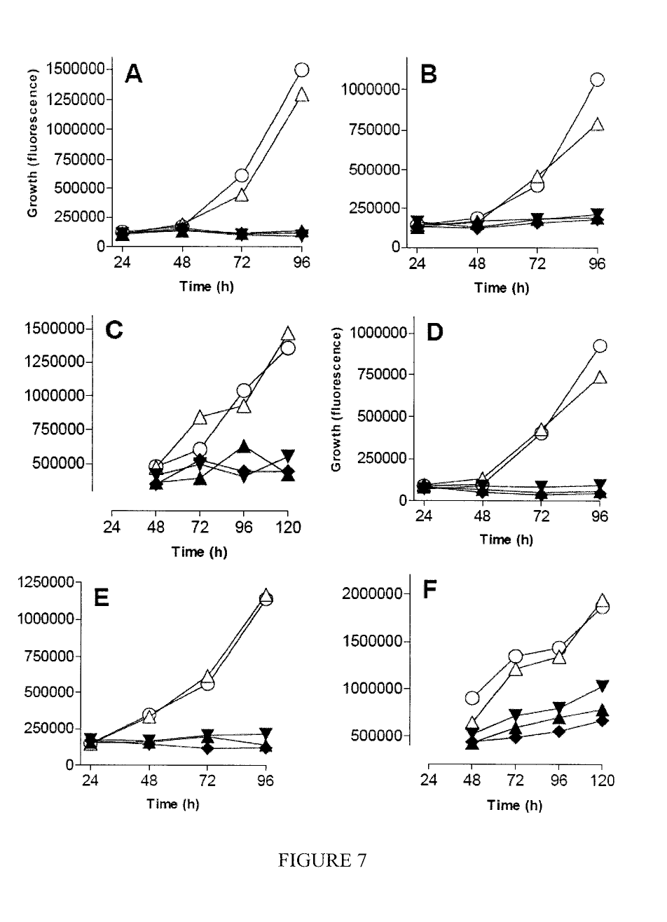

The growth inhibition in cells transfected with RRM1 and RRM2 siRNAs are shown in FIG. 7A-F. Cell growth was inhibited by the RRM1 siRNAs in the A549 (FIG. 7A), SK-MeI-5 (FIG. 7B) and MCF-7 cells (FIG. 7C), with the greatest effect on the A549 cells. Similarly, cell growth was inhibited by the RRM2 siRNAs in the A549 (FIG. 7D), SK-MeI-5 (FIG. 7E) and MCF-7 cells (FIG. 7F), with the greatest effect on the A549 and SK-MeI-5 cells. Minimal or no growth inhibition was observed with the non-specific siRNA control in all cell lines compared with growth of untransfected cells.

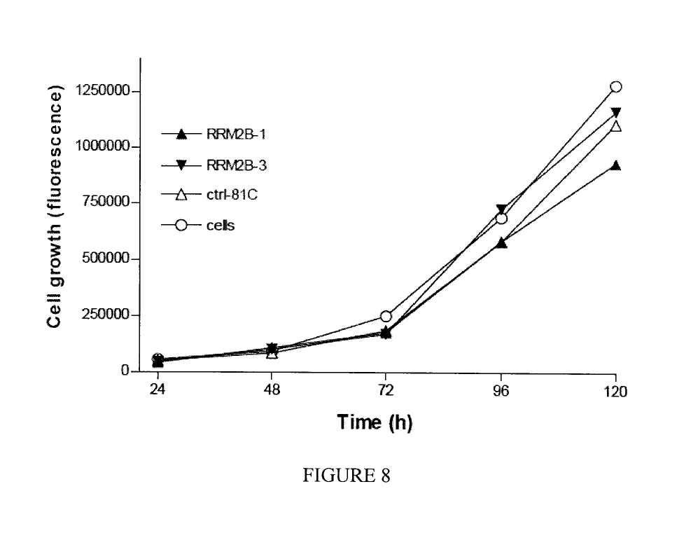

Growth inhibition in A549 cells by two siRNAs (SEQ ID NO: 4872 and 4873 (DNA), and SEQ ID NO: 5175 and 5176 (RNA)) targeting the ribonucleotide reductase 2B subunit (RRM2B; SEQ ID NO: 39) is shown in FIG. 8. The cells were transfected with 10 nM siRNA and growth inhibition followed up to 120 hours post-transfection. No inhibition of growth by the RRM2B siRNAs was observed compared with the control siRNA (SEQ ID NO: 4804 (DNA), and SEQ ID NO: 5301 (RNA)) or untransfected cells.

### Dose-Dependent Effect of RRM Knockdown on A549 Cell Growth

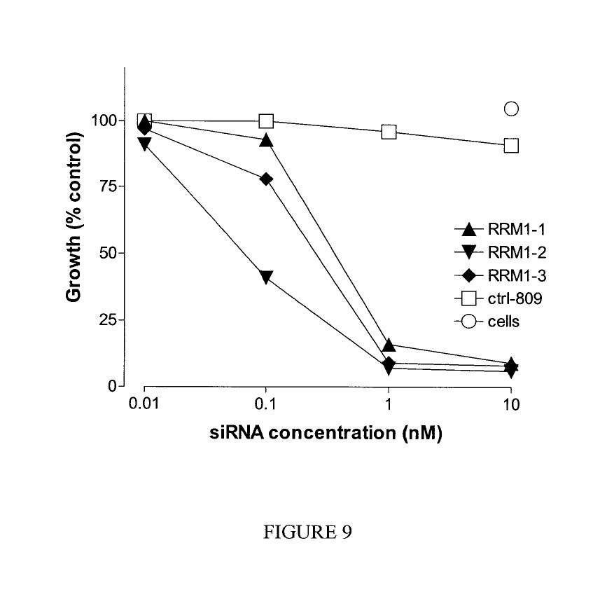

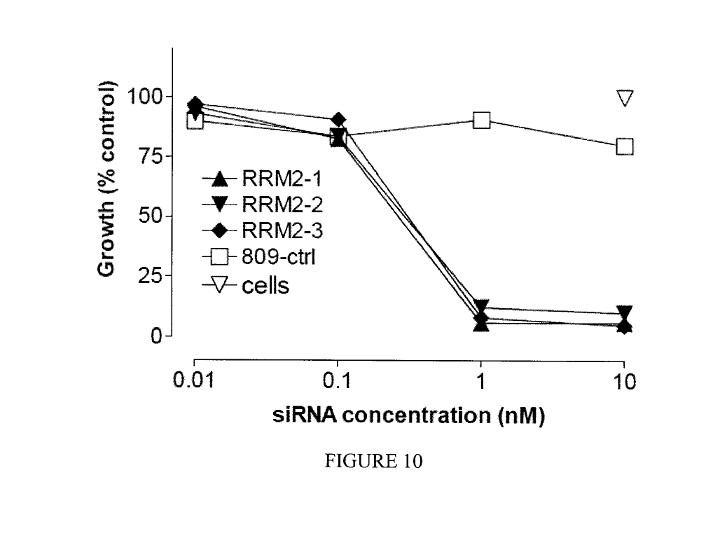

To determine the effect of different concentrations of RRM1 and RRM2 siRNAs on growth inhibition, A549 cells were reverse transfected with 0.01, 0.1, 1.0 and 10 nM siRNA (RRM1: SEQ ID NO: 4830-4832 (DNA, and SEQ ID NO: 5136-5138 (RNA); RRM2: SEQ ID NO: 5306-5308 (DNA), and SEQ ID NO: 5303-5305 (RNA)) and cell growth measured at 96 h post-transfection. Values were normalized to the control siRNA (SEQ ID NO: 4805 (DNA), and SEQ ID NO: 5302 (RNA)) at 0.01 nM. All three RRM1 siRNAs strongly inhibited growth at 1 nM, with RRM1-2 inhibiting growth by 50% at 0.1 nM (FIG. 9). Growth inhibition of RRM2 was achieved at 1 and 0.1 nM siRNA with all three RRM2 siRNAs tested (FIG. 10).

### Duration of RRM1 Knockdown in A549 Cells

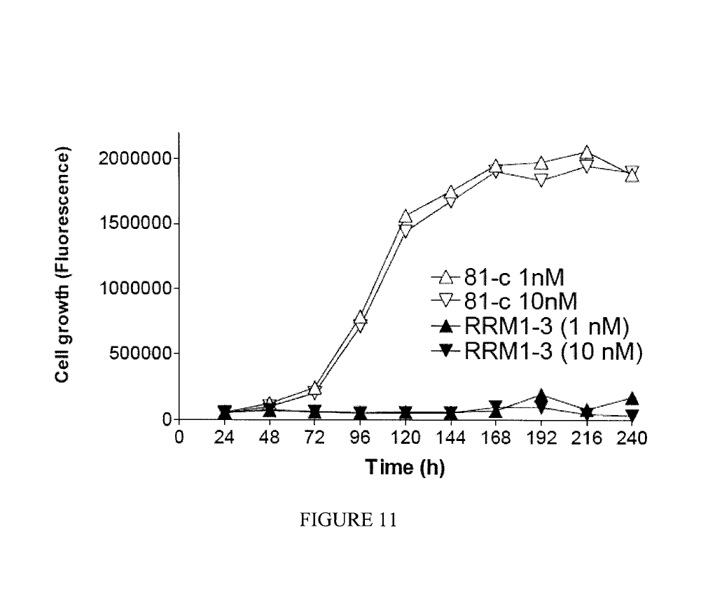

To determine the duration of the knock-down effect of RRM1-targeting siRNA, A549 cells were reverse transfected with 1 or 10 nM RRM1-3 (SEQ ID NO: 4832 (DNA, and SEQ ID NO: 5138 (RNA)) or control siRNA (SEQ ID NO: 4804 (DNA), and SEQ ID NO: 5301 (RNA)), and growth followed over time. Growth inhibition was measured daily as described above for up to ten days. RRM1-3-mediated RRM1 silencing inhibited growth for at least 10 days post-transfection at both 1 and 10 nM (FIG. 11).

Effect of siRNA Knockdown on Ribonucleotide Reductase RRM1 mRNA Level

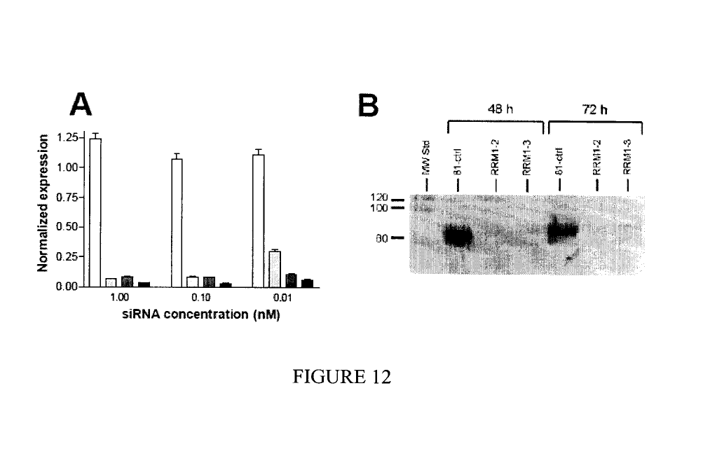

A549 cells were transfected with 10 nM RRM1-1 (SEQ ID NO: 4830 (DNA), and SEQ ID NO: 5136 (RNA); light grey bar in FIG. 12A), RRM1-2 (SEQ ID NO: 4831 (DNA), and SEQ ID NO: 5137 (RNA); dark grey bar in FIG. 12A), RRM1-3 (SEQ ID NO: 4832 (DNA), and SEQ ID NO: 5138 (RNA); black bar in FIG. 12A) or control siRNA (SEQ ID NO: 4804 (DNA), and SEQ ID NO: 5301 (RNA)) (open bar in FIG. 12A) and relative mRNA levels quantified 24 h post-transfection. RNA was purified from cells using Trizol® extraction as per the manufacturer's protocol (Invitrogen). cDNA was prepared by digesting approximately 200 ng purified RNA with DNAse I (Invitrogen) for 15 minutes at room temperature, followed by 15 minutes at 65° C. in the presence of 25 mM EDTA. 30 ng random primers were added and incubated for 10 minutes at room temperature, 1 minute on ice; Superscript III polymerase (Invitrogen) was added in presence of 5 nM DTT (Sigma, St Louis, Mo.) and 1 mM deoxyribonucleotides (Invitrogen), and the reaction incubated at 25° C. for 5 minutes, followed by an hour at 55° C. cDNA was diluted 1:3 in 10 mM Tris pH 7.0, and quantitative RT-PCR carried out on an ABI Prism 7900HT (Applied Biosciences, Inc., Foster City, Calif.) using FastStart TaqMan SYBRGreen (Roche, Basel, Switzerland) and specific primers at 360 nM for RRM1 and RRM2 and the housekeeping genes HPRT and Lamin. Primer sequence pairs used for RRM1, RRM2, HPRT and lamin are given in SEQ ID NO: 4881 and 4882; 4883 and 4884; 4885 and 4886; 4887 and 4888 respectively. As little as 0.01 nM RRM1-1 siRNA effectively silenced RRM1 mRNA expression as shown in FIG. 12A.

Effect of siRNA Knockdown on Ribonucleotide Reductase Protein Levels

Cell lysates were prepared from A549 cells 48 and 72 h post-transfection with 10 nM RRM1-specific siRNA (SEQ ID NO: 4830-4832 (DNA), and SEQ ID NO: 5136-5138 (RNA)) or control siRNA (SEQ ID NO: 4804 (DNA), and SEQ ID NO: 5301 (RNA)). Following Western blotting onto Immobilon-P PVDF filer (Millipore, Bedford Mass.), membranes were probed with a goat anti-human polyclonal antibody specific for RRM1 (Santa Cruz Biotechnology, Inc., Santa Cruz Calif.), at a concentration of 0.4 mg/ml. HRP-conjugated donkey anti-goat IgG (80 mg/ml; Santa Cruz) was used as a secondary antibody, and signal was detected with an ECL Plus Western blotting Detection System (GE Healthcare, UK) using a Typhoon Scanner (Molecular Dynamics, GE Healthcare, UK). FIG. 12B shows siRNA-mediated reduction in protein levels in the cells up to 72 hours post transfection compared with the control siRNA.

### Effect of p53 Status on Ribonucleotide Reductase Knockdown

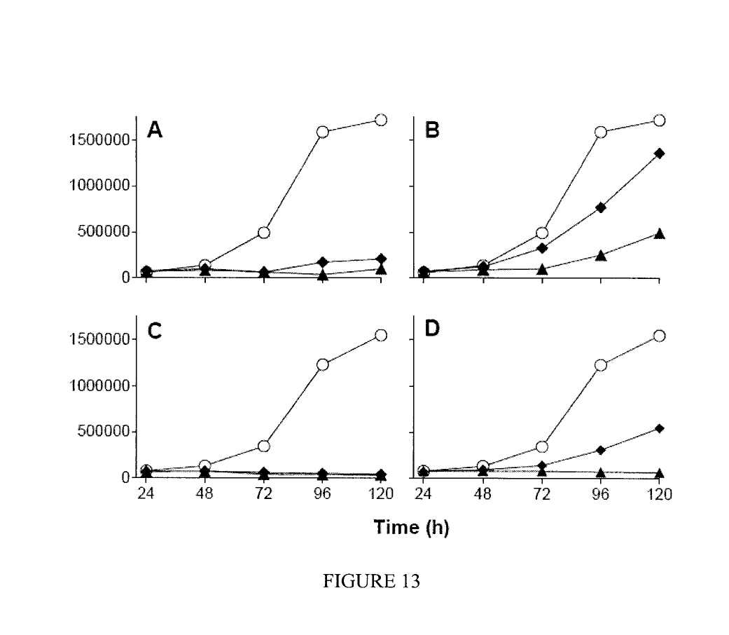

To determine the effect of the p53-status of cells on RRM1 and RRM2 knockdown, growth inhibition of wild type (wt) HCT-116 and p53 null HCT-116 cells by RRM1 and RRM2-targeted siRNA were determined. Cells were reverse transfected with 1.0 and 10 nM RRM1-3 (SEQ ID NO: 4832 (DNA), and SEQ ID NO: 5138 (RNA)), 1.0 and 10 nM RRM2-2 (SEQ ID NO: 5307 (DNA), and SEQ ID NO: 5304 (RNA)) or 10 nM control siRNA (SEQ ID NO: 4804 (DNA), and SEQ ID NO: 5301 (RNA)), and growth measured daily for up to 4 days post transfection. RRM1 knockdown was equally growth inhibitory in p53 wt HCT-116 (FIG. 13A) and p53 null HCT-116 (FIG. 13C). RRM2 knockdown was more growth inhibitory in p53 null HCT-116 (FIG. 13D) than p53 wt HCT-116 (FIG. 13B).

### Silencing of RRM1 Leads to Induction of Apoptosis

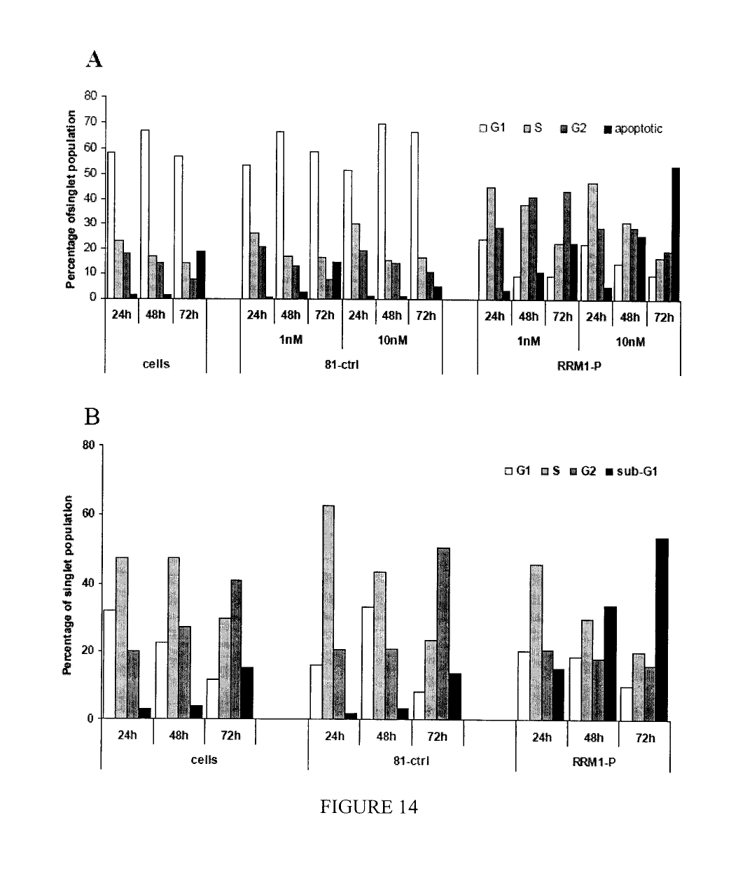

The mechanism by which RRM1 silencing inhibited growth was investigated using flow cytometric cell cycle analysis techniques. A549 cells were reverse transfected with a pool of RRM1 siRNAs (SEQ ID NO: 4830-4832 (DNA), and SEQ ID NO: 5136-5138 (RNA)) or control siRNA (SEQ ID NO: 4804 (DNA), and SEQ ID NO: 5301 (RNA)) at 1 or 10 nM final siRNA concentration (FIG. 14A). The transfection was also repeated with 10 nM pooled RRM1 siRNA in the presence of 10 nM chemotherapeutic nucleoside analog cytarabine (FIG. 14B) and the formation of a singlet population followed over 72 hours. As seen in FIG. 14A, there was a rapid arrest of the cell cycle with an early accumulation of cells in S phase in the first 24 h post transfection. This initial S-phase arrest was followed by the induction of apoptosis, as defined by DNA content less than G1 cells, and by 72 h the majority of the cells had a sub-G1 DNA content. There was a minor increase in apoptotic RRM1 siRNA-treated A549 cells subsequently exposed to 10 nM cytarabine (FIG. 14B). This suggests that the inhibition of DNA synthesis and induction of apoptosis are the major consequences of RRM1 knockdown rather than a sensitization to chemotherapeutic nucleoside analogs. Cell cycle arrest is indicated by a gradual accumulation over time of cells in S-phase, and this is followed by the appearance of cells in the apoptotic sub-G1 population (FIG. 14B).

### Effect on Tumor Growth of Transfection of Cells Pre-Implantation

To determine the effect of RRM1 knockdown on tumor growth, A549 cells (human lung cancer cell line; ATCC No. CCL-185) were reverse-transfected prior to implantation in CD-1 nude mice. In 175 cm² culture flasks, 10×10⁶ A549 cells (in RPMI supplemented with 10% fetal calf serum) were added to pre-made lipoplexes. To make lipoplexes, Lipofectamine RNAiMAX in serum-free RPMI was added to an equal volume of serum-free RPMI containing siRNA (RRM1-2; SEQ ID NO: 4831 (DNA), and SEQ ID NO: 5137 (RNA)) and a control siRNA (SEQ ID NO: 4804 (DNA), and SEQ ID NO: 5301 (RNA)). After addition of cells, the final concentration of siRNA in the flask was 10 nM. Untransfected cells served as a further control. Flasks were incubated at 37° C. for 24 h to allow transfection to proceed.

The cells were harvested by trypsinization 24 h after the transfection, washed twice in PBS and viable cells were counted. Equal numbers of cells (1×10⁷) were inoculated into CD-1 nude mice (n=7-9). Briefly, 5 to 7 weeks old nude mice were subcutaneously (s.c.) injected in the right flank with A549 cells in 100 μl PBS. Tumor growth was monitored 3 times per week by measuring tumor sizes using a digital caliper. Tumor volume was expressed in mm using the formula: V=a²×b×0.52, where a and b represent the minimum and maximum tumor diameter, respectively. Mean tumor volumes calculated from each measurement were then plotted in a standard graph to compare the tumor growth of transfected cells to that of control.

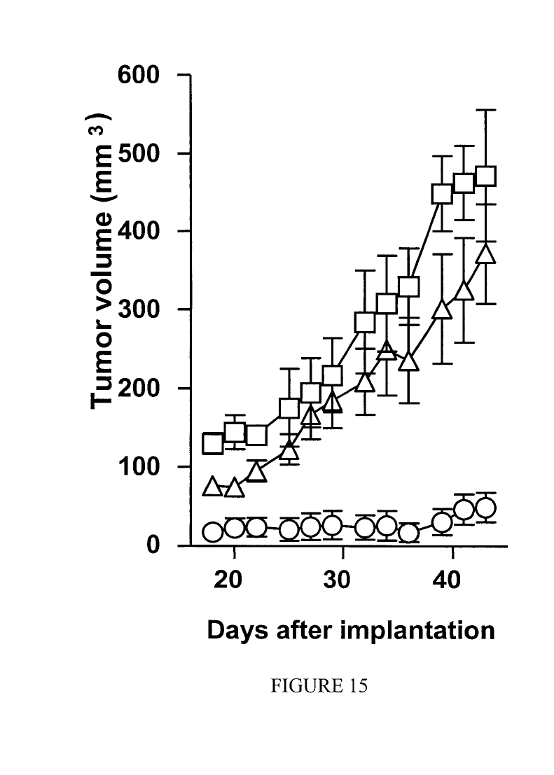

Tumors developed in all mice injected with untransfected cells or cells treated with the control siRNA. There was no significant difference in tumor growth rate between these two control groups (FIG. 15: open squares and open triangles). Only two of eight mice in the RRM-1 siRNA-treated group had measurable tumors and the tumor growth rate was reduced dramatically compared to the control groups (FIG. 15: open circles). Six weeks after the cell inoculation, the average tumor size from RRM-1 siRNA transfectants (49.1±18.4 mm³) was significantly smaller than those of the tumors from untransfected cells (470.9±84.5 mm³; P<0.001; Student's t-test) and the control siRNA transfectants (370.7±63.8 mm³; P<0.001).

Treatment of Established Tumors with RRM1-Targeting siRNA

A549 cells (6−8×10⁶ cells) were s.c. inoculated into CD-1 nude mice as described above. When tumors reached an average volume of approximately 100 mm³, the tumor-bearing mice were randomly assigned into three different treatment groups (RRM1 siRNA, control siRNA and PBS) with 5 to 6 mice in each group. There were no significant differences in tumor volumes among groups prior to the initiation of treatments. Mice were anesthetized by gaseous isoflurane and 50 μl siRNA or PBS was injected directly into tumors using a 30-gauge needle. Two experiments were conducted. In the first experiment (IT#1), the animals received a dose of 50 μg of siRNA in each intratumoral (i.t.) injection and the injections were performed 3 times per week for 2 weeks. In the second experiment (IT#2), 25 μg siRNA was used and mice were injected 3 times per week for 3 weeks. The tumor volume was determined immediately before each injection by perpendicular measurements of the shortest and longest diameters as described above.

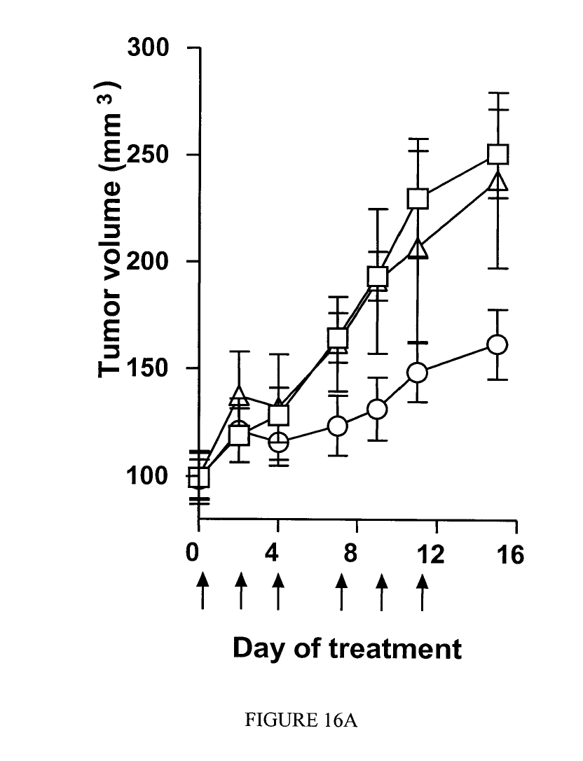

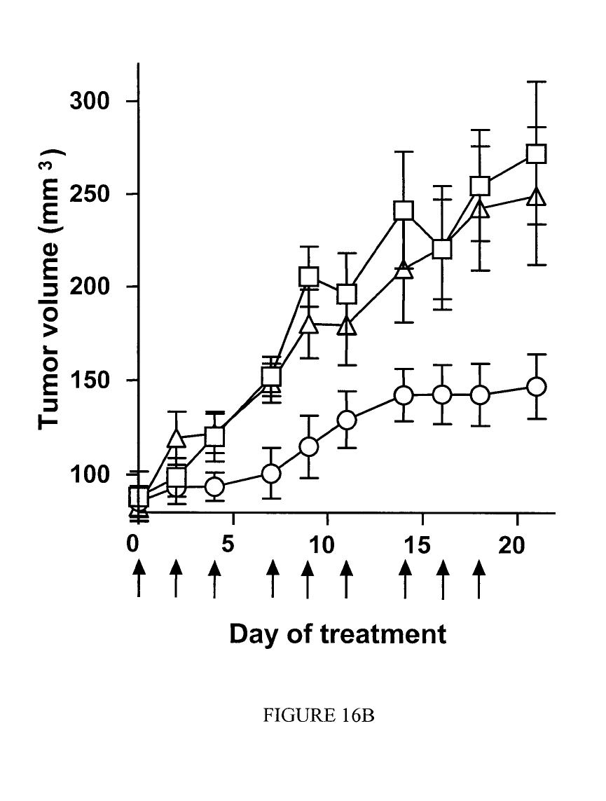

As shown in FIGS. 16A and 16B, in both experiments i.t. injection of RRM1-targeting siRNA led to a significant inhibition in the tumor growth. In the first experiment, on day 15 after treatment initiation (4 days after the last injection), the average tumor volume in animals treated with RRM1 siRNA was 161.7±16.1 mm³, which was significantly smaller than those in PBS and control siRNA treated mice (251.1±20.9 mm³ and 238.7±41.4 mm³ respectively; FIG. 16A). In the second experiment, animals treated with RRM1 siRNA showed inhibition of tumor growth by 46% and 41% compared to PBS and control siRNA group respectively (FIG. 16B). On day 21 post-treatment initiation (3 days after the last injection), the tumor volume in RRM1 siRNA treated mice was 147±17.2 mm³ whereas the tumor volume in PBS- and control siRNA treated animals was 271.9±38.3 mm³ and 248.9±37.3 mm³ respectively.

### EXAMPLE 9

### siRNA Mediated Silencing of Transporter Genes

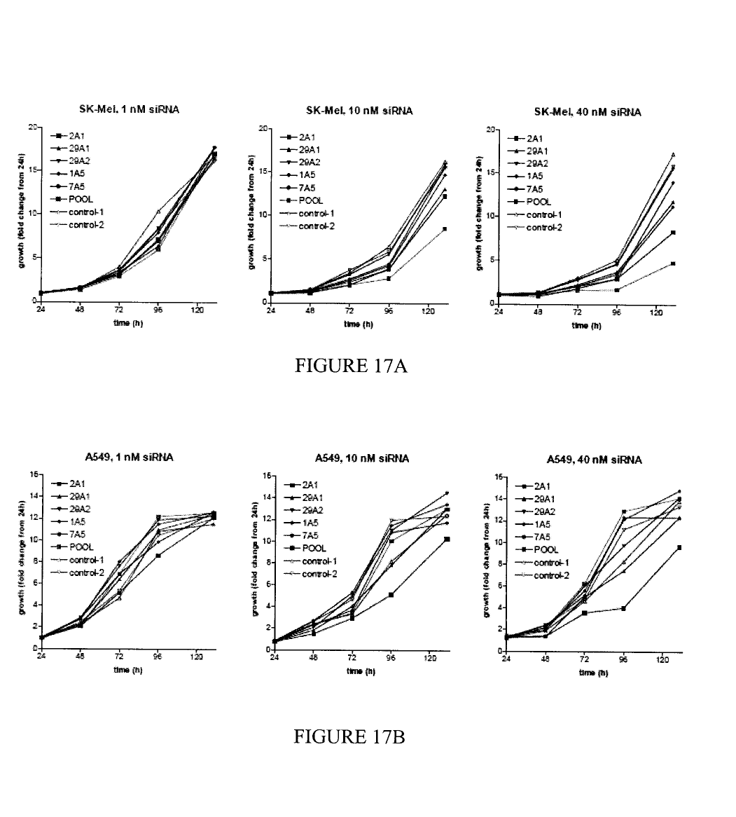

The effect of siRNA-mediated transporter gene knockdown on cancer cell growth and mRNA levels was tested as detailed below. A pool of three siRNAs targeting SLC1A5 (ASCT2; SEQ ID NO: 4889-4891 (DNA), and SEQ ID NO: 5184-5186 (RNA)), SLC2A1 (GLUT1; SEQ ID NO: 4892-4894 (DNA), and SEQ ID NO: 5187-5189 (RNA)), SLC7A5 (LAT1; SEQ ID NO: 4901-4903 (DNA), and SEQ ID NO: 5196-5198 (RNA)), SLC29A1 (ENT1; SEQ ID NO: 4931-4933 (DNA), and SEQ ID NO: 5226-5228 (RNA)) or SLC29A2 (ENT2; SEQ ID NO: 4934-4936 (DNA), and SEQ ID NO: 5229-5231 (RNA)), or a pool of one siRNA against each of the five targets (SEQ ID NO: 4891, 4893, 4901, 4931 and 4934 (DNA), and SEQ ID NO: 5186, 5186, 5196, 5226, 5229 (RNA)), or control siRNAs (control-1: SEQ ID NO: 4804 (DNA), and SEQ ID NO: 5301 (RNA); control-2: SEQ ID NO: 4805 (DNA), and SEQ ID NO: 5302 (RNA)) were tested to determine down-regulation of target genes in A549 (human lung carcinoma cell line; ATCC Cat. No. CCL-185), and SK-MeI-5 (human melanoma cell line; ATCC Cat. No. HTB-70) cells. In 24-well culture plates, (2×10⁴) cells were transfected with a final concentration of 1, 10 or 40 nM siRNA, using Lipofectamine 2000 transfection reagent (Invitrogen, Carlsbad Calif.). Transfections contained three siRNAs against each individual target (SLC2A1, SLC29A1, SLC29A2, SLC1A5, or SLC7A5) or one siRNA against each of the 5 targets (pool). Controls used were either control-1, or both control-1 and control-2 together. At 24 hour-intervals post-transfection, plates were harvested by removing medium and freezing at −80° C. The next day, plates were removed and DNA content in wells was quantified with the CyQuant™ cell proliferation kit (Invitrogen, Carlsbad, Calif.). As shown in FIG. 17, the growth of SK-MeI-5 (FIG. 17A) and A549 (FIG. 17B) cells transfected with 10 nM siRNAs targeting the transporters listed above was markedly reduced compared with controls.

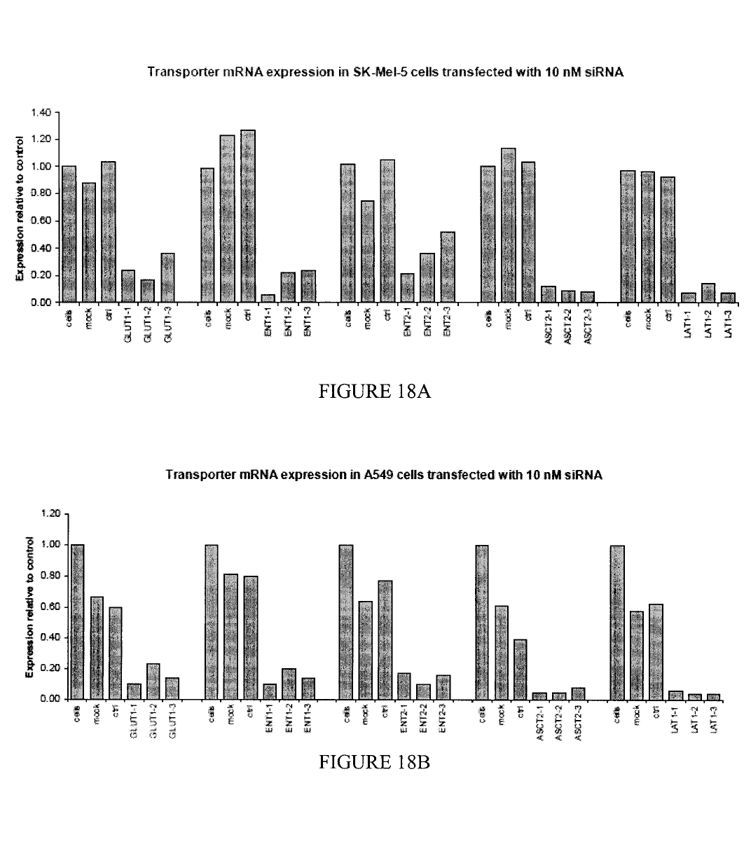

At 48 h post-transfection, RNA was extracted and real-time PCR was carried out to determine relative levels of transporter mRNA. In each case, transfection of the specific siRNA resulted in a decrease in the cognate mRNA as shown in FIGS. 18A and B, whereas control siRNA had no effect.

It is known that siRNA can produce non-specific concentration-dependent effects on mammalian gene expression (Scherer and Rossi, Nature Biotechnol. 21:1457-1465, 2003; Persengiev et al., RNA 10:12-18, 2004). These off-target effects can be minimized by selecting siRNAs with unique sequences, and using them at subnanomolar to nanomolar concentrations. In the above experiments, siRNA concentration is optimized for downregulation and nonspecific effects. Non-specific effects are assessed by microarray-based expression profiling.

### EXAMPLE 10

### siRNA-Mediated Inhibition of Cancer Cell Growth

siRNA-mediated inhibition of cancer cell growth was determined in A549 cells as follows. A549 cells (human lung cancer cell line; ATCC No. CCL-185) were cultured in RPMI 1640 medium with 10% v/v heat-inactivated fetal calf serum. Cells were seeded at a density of 5×10³ cells/cm² in multi-well tissue culture plates. Stealth™ siRNA duplexes (Invitrogen) targeting genes involved in cancer energy and metabolism pathways and listed in Table 9, were transfected at 10 nM into cells using Lipofectamine™ RNAiMAX transfection reagent (Invitrogen). Non-specific siRNA (SEQ ID NO: 4804 and 4805 (DNA), and SEQ ID NO: 5301 and 5302 (RNA)) were used as controls. The cellular DNA content was measured using a SYBR Green I-based fluorometric assay at 96 hours after transfection. The cells were harvested and frozen at −80° C. overnight. Cells were thawed and cell lysis buffer (phosphate-buffered saline (PBS) containing 1% Triton X-100, pH 7) and SYBR Green I (1:10⁵ v/v; Invitrogen) was added to the wells and incubated overnight in the dark at 4° C. The following day, cell lysates were mixed thoroughly, and DNA fluorescence was measured with a Wallac Victor² plate reader (Turku, Finland) set at an excitation frequency of 485 nm and measuring emission at 535 nm.

Three different siRNAs were tested for each target gene. The average fluorescence units from three replicate wells, representing the amount of DNA per well, were used to determine the effect on growth inhibition. The results are given in Table 10 (energy and metabolism gene targets) and Table 11 (transporter gene targets).

TABLE 10
Effect of siRNA-mediated RNAi on growth of A549 cells (energy and
metabolism gene targets)

| | | | Antisense | Antisense | Growth |
|---|---|---|---|---|---|
| | | SEQ ID NO: of | DNA | RNA | Inhibition |
| | Target gene | target poly- | SEQ ID | SEQ ID | in A549 |
| Target gene | nomenclature | nucleotide | NO: | NO: | cells |
| Hypoxia | ARNT | 3 | 4806 | 5112 | − |
| induced factor 1 | | | 4807 | 5113 | + |
| beta | | | 4808 | 5114 | − |
| Hexose-6- | H6PD | 14 | 4809 | 5115 | ++ |
| phosphate | | | 4810 | 5116 | − |
| dehydrogenase | | | 4811 | 5117 | − |
| (G6PDH) | | | | | |
| Hexokinase II | HK-II | 5 | 4812 | 5118 | − |
| | | | 4813 | 5119 | ++ |
| | | | 4814 | 5120 | − |
| Lactate | LDHA/5 | 9 | 4815 | 5121 | − |
| dehydrogenase 5 | | | 4816 | 5122 | + |
| | | | 4817 | 5123 | − |
| Phosphofructo- | PFKL | 11 | 4818 | 5124 | − |
| kinase 1 - liver | | | 4819 | 5125 | − |
| | | | 4820 | 5126 | − |
| Phosphate | PRPS1 | 18 | 4821 | 5127 | − |
| ribosyl | | | 4822 | 5128 | − |
| pyrophosphate | | | 4823 | 5129 | + |
| synthase | | | | | |
| Carbamoyl | CPS1 | 20 | 4824 | 5130 | ++ |
| phosphate | | | 4825 | 5131 | − |
| synthetase 1 | | | 4826 | 5132 | − |
| Carbamoyl | CAD | 21 | 4827 | 5133 | + |
| phosphate | | | 4828 | 5134 | − |
| synthetase 2 | | | 4829 | 5135 | + |
| Ribonucleotide | RRM1 | 22 | 4830 | 5136 | ++ |
| reductase | | | 4831 | 5137 | ++ |
| subunit 1 | | | 4832 | 5138 | ++ |
| Thymidylate | TYMS | 23 | 4833 | 5139 | + |
| synthase | | | 4834 | 5140 | ++ |
| | | | 4835 | 5141 | ++ |
| Dihydrofolate | DHFR | 25 | 4836 | 5142 | + |
| reductase | | | 4837 | 5143 | − |
| | | | 4838 | 5144 | + |
| Adenylate | AK1 | 26 | 4839 | 5145 | − |
| kinase | | | 4840 | 5146 | + |
| | | | 4841 | 5147 | − |
| NAD synthase | NADSYN | 27 | 4842 | 5148 | + |
| | | | 4843 | 5149 | − |
| | | | 4844 | 5150 | − |
| FAD synthase | FLAD1 | 28 | 4845 | 5151 | + |
| | | | 4846 | 5152 | − |
| | | | 4847 | 5153 | − |
| Cytochrome | COX5A | 31 | 4848 | 5154 | ++ |
| oxidase | | | 4849 | 5155 | − |
| | | | 4850 | 5156 | − |
| ATP synthase | ATP5B | 32 | 4851 | 5157 | − |
| | | | 4852 | 5158 | − |
| | | | 4853 | 5159 | + |
| Fatty acid | FASN | 36 | 4854 | 5160 | − |
| synthase | | | 4855 | 5161 | + |
| | | | 4856 | 5162 | − |
| ATP citrate | ACLY | 37 | 4857 | 5163 | + |
| lyase | | | 4858 | 5164 | − |
| | | | 4859 | 5165 | − |
| ANT2 | SLC25A5 | 34 | 4860 | 5166 | + |
| | | | 4861 | 5167 | − |
| | | | 4862 | 5168 | − |
| ANT3 | SLC25A6 | 35 | 4863 | 5169 | ++ |
| | | | 4864 | 5170 | − |
| | | | 4865 | 5171 | − |
| Cytochrome | UQCRC2 | 30 | 4866 | 5172 | − |
| reductase | | | 4867 | 5173 | − |
| | | | 4868 | 5174 | + |
| Ribonucleotide | RRM2 | 38 | 5306 | 5303 | ++ |
| reductase | | | 5307 | 5304 | ++ |
| subunit 2 | | | 5308 | 5305 | ++ |
| Ribonucleotide | RRM2B | 39 | 4872 | 5175 | − |
| reductase p53 | | | 4873 | 5176 | − |
| inducible | | | 4874 | 5177 | − |
| CTP synthase | CTPS | 40 | 4875 | 5178 | ++ |
| | | | 4876 | 5179 | − |
| | | | 4877 | 5180 | + |
| Inosine | IMPDH2 | 41 | 4878 | 5181 | − |
| monophosphate | | | 4879 | 5182 | ++ |
| dehydrogenase 2 | | | 4880 | 5183 | + |
| Deoxycytidine | DCK | 42 | 4986 | 5281 | − |
| kinase | | | 4987 | 5282 | − |
| | | | 4988 | 5283 | − |
| Thymidine | TK1 | 43 | 4989 | 5284 | − |
| kinase 1 | | | 4990 | 5285 | − |
| | | | 4991 | 5286 | − |
| Thymidine | TK2 | 44 | 4992 | 5287 | − |
| kinase 2 | | | 4993 | 5288 | − |
| | | | 4994 | 5289 | − |
| Deoxy- | DGUOK | 45 | 4995 | 5290 | − |
| guanosine | | | 4996 | 5291 | − |
| kinase | | | 4997 | 5292 | + |
| Uridine | UMPS | 46 | 4998 | 5293 | − |
| monophosphate | | | 4999 | 5294 | + |
| synthase | | | 5000 | 5295 | − |
| Dihydro- | DPYD | 47 | 5001 | 5296 | − |
| pyrimidine | | | 5002 | 5297 | − |
| dehydrogenase | | | 5003 | 5298 | − |

−: no growth inhibition with any siRNA
+: inhibits by at least 50%
++: inhibits by at least 75%

TABLE 11
Effect of siRNA-mediated RNAi on growth of A549 or SK-Mel-5 cells
(transporter gene targets)

| | | | Antisense | Antisense | Growth |
|---|---|---|---|---|---|
| | | SEQ ID NO: | DNA | RNA | Inhibition in |
| | Target gene | of target | SEQ ID | SEQ ID | A549 cells by |
| Target gene | nomenclature | polynucleotide | NO: | NO: | 10 nM siRNA |
| GLUT1 | SLC2A1 | 48 | 4892 | 5187 | + |
| | | | 4893 | 5188 | ++ |
| | | | 4894 | 5189 | − |
| GLUT5 | SLC2A5 | 50 | 4895 | 5190 | − |
| | | | 4896 | 5191 | − |
| | | | 4897 | 5192 | ++ |
| GLUT12 | SLC2A12 | 51 | 4898 | 5193 | − |
| | | | 4899 | 5194 | − |
| | | | 4900 | 5195 | − |
| ENT1 | SLC29A1 | 52 | 4931 | 5226 | ++ |
| | | | 4932 | 5227 | − |
| | | | 4933 | 5228 | + |
| ENT2 | SLC29A2 | 53 | 4934 | 5229 | − |
| | | | 4935 | 5230 | − |
| | | | 4936 | 5231 | − |
| ASCT2 | SLC1A5 | 60 | 4889 | 5184 | − |
| | | | 4890 | 5185 | − |
| | | | 4891 | 5186 | − |
| 4Fhc; CD98 | SLC3A2 | 61 | 4907 | 5202 | − |
| | | | 4908 | 5203 | ++ |
| | | | 4909 | 5204 | − |
| LAT1 | SLC7A5 | 63 | 4901 | 5196 | − |
| | | | 4902 | 5197 | + |
| | | | 4903 | 5198 | − |
| xCT | SLC7A11 | 64 | 4904 | 5199 | − |
| | | | 4905 | 5200 | ++ |
| | | | 4906 | 5201 | − |
| SMVT1 | SLC5A6 | 68 | 4910 | 5205 | − |
| | | | 4911 | 5206 | − |
| | | | 4912 | 5207 | − |
| ThTr1 | SLC19A2 | 70 | 4913 | 5208 | − |
| | | | 4914 | 5209 | ND |
| | | | 4915 | 5210 | ND |
| SVCT2 | SLC23A2 | 73 | 4916 | 5211 | − |
| | | | 4917 | 5212 | − |
| | | | 4918 | 5213 | − |
| CIC | SLC25A1 | 74 | 4925 | 5220 | − |
| | | | 4926 | 5221 | ++ |
| | | | 4927 | 5222 | − |
| DIC | SLC25A10 | 75 | 4928 | 5223 | ++ |
| | | | 4929 | 5224 | − |
| | | | 4930 | 5225 | − |
| NHE1 | SLC9A1 | 76 | 4919 | 5214 | + |
| | | | 4920 | 5215 | − |
| | | | 4921 | 5216 | − |
| MCT1 | SLC16A1 | 77 | 4922 | 5217 | + |
| | | | 4923 | 5218 | − |
| | | | 4924 | 5219 | ++ |
| PMCA1 | ATP2B1 | 78 | 4937 | 5232 | − |
| | | | 4938 | 5233 | − |
| | | | 4939 | 5234 | − |
| PMCA2 | ATP2B2 | 79 | 4940 | 5235 | ++ |
| | | | 4941 | 5236 | + |
| | | | 4942 | 5237 | − |
| PMCA3 | ATP2B3 | 80 | 4943 | 5238 | − |
| | | | 4944 | 5239 | − |
| | | | 4945 | 5240 | + |
| PMCA4 | ATP2B4 | 81 | 4946 | 5241 | − |
| | | | 4947 | 5242 | − |
| | | | 4948 | 5243 | − |
| ATP6V1A | ATP6V1A | 82 | 4949 | 5244 | − |
| (H⁺ ATPase) | | | 4950 | 5245 | − |
| | | | 4951 | 5246 | + |
| ATP6V1B2 | ATP6V1B2 | 83 | 4952 | 5247 | − |
| (H⁺ ATPase | | | 4953 | 5248 | ++ |
| lysosomal | | | 4954 | 5249 | − |
| 70 kDa, V1 | | | | | |
| subunit A) | | | | | |
| ATP6V1C1 | ATP6V1C1 | 84 | 4955 | 5250 | − |
| (H⁺ ATPase | | | 4956 | 5251 | − |
| lysosomal | | | 4957 | 5252 | − |
| 42 kDa, V1 | | | | | |
| subunit C1) | | | | | |
| ATP6V1E1 | ATP6V1E1 | 85 | 4958 | 5253 | − |
| (H⁺ ATPase | | | 4959 | 5254 | − |
| lysosomal | | | 4960 | 5255 | − |
| 31 kDa, V1 | | | | | |
| subunit E1) | | | | | |
| ATP6V1F | ATP6V1F | 86 | 4961 | 5256 | − |
| (H⁺ ATPase | | | 4962 | 5257 | − |
| lysosomal | | | 4963 | 5258 | − |
| 14 kDa, V1 | | | | | |
| subunit F) | | | | | |
| ATP7A | ATP7A | 87 | 4964 | 5259 | − |
| (Cu²⁺ ATPase, | | | 4965 | 5260 | − |
| alpha | | | 4966 | 5261 | − |
| polypeptide) | | | | | |
| ATP7B | ATP7B | 88 | 4967 | 5262 | + |
| (Cu²⁺ ATPase, | | | 4968 | 5263 | − |
| beta | | | 4969 | 5264 | − |
| polypeptide) | | | | | |
| Multidrug | ABCC4 | 90 | 4970 | 5265 | − |
| resistance | | | 4971 | 5266 | − |
| protein 4 | | | 4972 | 5267 | − |
| (MRP4) | | | | | |
| Multidrug | ABCC5 | 91 | 4973 | 5268 | − |
| resistance | | | 4974 | 5269 | + |
| protein 5 | | | 4975 | 5270 | ++ |
| (MRP5) | | | | | |
| ABC | ABCB1 | 89 | 4976 | 5271 | − |
| transporter 1; | | | 4977 | 5272 | − |
| MDR-1 | | | 4978 | 5273 | − |
| | | | 4979 | 5274 | − |
| | | | 4980 | 5275 | − |

−: no growth inhibition
+: inhibits by at least 50%
++: inhibits by at least 75%

Effect of siNA-mediated RNAi on Level of mRNA on Target Transcript in A549 Cells

A549 cells were transfected as described before with 0.1, 1.0 and 10 nM siRNA as listed in Tables 10 and 11 and the relative mRNA levels quantified 24 h post-transfection. RNA was purified from cells using Trizol® extraction as per the manufacturer's protocol (Invitrogen). cDNA was prepared by digesting approximately 200 ng purified RNA with DNAse I (Invitrogen) for 15 minutes at room temperature, followed by 15 minutes at 65° C. in the presence of 25 mM EDTA. 30 ng random primers were added and incubated for 10 minutes at 65° C., 1 minute on ice; Superscript® III polymerase (Invitrogen) was added in presence of 5 nM DTT (Invitrogen) and 1 mM deoxyribonucleotides (Invitrogen), and the reaction incubated at 25° C. for 5 minutes, followed by an hour at 50° C. cDNA was diluted 1:3 in 10 mM Tris pH 7.0, and quantitative RT-PCR carried out on an ABI Prism 7900HT (Applied Biosciences, Inc., Foster City, Calif.) using FastStart TaqMan SYBRGreen (Roche, Basel, Switzerland) or Platinum SYBR Green (Invitrogen) and 360 nM of the primers specific for gene targets and housekeeping genes HPRT and Lamin. Reduction in target mRNA levels following siRNA transfection are given in Tables 12 and 13, together with the gene specific primer sequences. Primer sequences used for the housekeeping genes HPRT and Lamin are given in SEQ ID NO: 4885-4888.

TABLE 12
Effect of siRNA-mediated RNAi on level of mRNA on energy and
metabolism gene target transcripts in A549 cells

| | RT-PCR | Antisense | Antisense | | | |
|---|---|---|---|---|---|---|
| | primers | DNA | RNA | | | |
| | SEQ ID | SEQ ID | SEQ ID | | | 10 |
| Target gene | NO: | NO: | NO: | 0.1 nM | 1 nM | nM |
| ARNT | 5004; | 1686 | 5112 | − | − | ND |
| | 5005 | 1687 | 5113 | − | − | ND |
| | | 1688 | 5114 | + | ++ | ND |
| H6PD | 5006; | 1689 | 5115 | − | ++ | ND |
| | 5007 | 1690 | 5116 | ++ | ++ | ND |
| | | 1691 | 5117 | − | ++ | ND |
| HK-II | 5008; | 1692 | 5118 | − | − | ND |
| | 5009 | 1693 | 5119 | − | − | ND |
| | | 1694 | 5120 | − | − | ND |
| LDHA-5 | 5010; | 1695 | 5121 | ++ | ++ | ND |
| | 5011 | 1696 | 5122 | − | − | ND |
| | | 1697 | 5123 | ++ | + | ND |
| PFKL | 5012; | 1698 | 5124 | + | + | ND |
| | 5013 | 1699 | 5125 | − | ++ | ND |
| | | 1700 | 5126 | + | ++ | ND |
| PRPS1 | 5014; | 3533 | 5127 | − | ++ | ND |
| | 5015 | 3534 | 5128 | ++ | ++ | ND |
| | | 3535 | 5129 | ++ | +++ | ND |
| CPS1 | 5016; | 3536 | 5130 | ++ | +++ | +++ |
| | 5017 | 3537 | 5131 | ++ | +++ | +++ |
| | | 3538 | 5132 | ++ | ++ | +++ |
| CAD | 5018; | 3539 | 5133 | − | − | − |
| | 5019 | 3540 | 5134 | − | − | − |
| | | 3541 | 5135 | − | − | − |
| RRM1 | 5881; | 3542 | 5136 | + | ++ | ND |
| | 5882 | 3543 | 5137 | ++ | ++ | ND |
| | | 3544 | 5138 | ++ | ++ | ND |
| TYMS | 5020; | 3545 | 5139 | ++ | ++ | +++ |
| | 5021 | 3546 | 5140 | + | ++ | +++ |
| | | 3547 | 5141 | ++ | ++ | +++ |
| DHFR | 5022; | 3548 | 5142 | ++ | ++ | +++ |
| | 5023 | 3549 | 5143 | + | +++ | ++ |
| | | 3550 | 5144 | ++ | ++ | +++ |
| AK1 | 5024; | 3551 | 5145 | ++ | − | ND |
| | 5025 | 3552 | 5146 | + | ++ | ND |
| | | 3553 | 5147 | ++ | ++ | ND |
| NADSYN | 5026; | 3554 | 5148 | ++ | ++ | ND |
| | 5027 | 3555 | 5149 | ++ | ++ | ND |
| | | 3556 | 5150 | ++ | ++ | ND |
| FLAD1 | 5028; | 3557 | 5151 | ++ | ++ | ND |
| | 5029 | 3558 | 5152 | − | ++ | ND |
| | | 3559 | 5153 | ++ | ++ | ND |
| COX5A | 5030; | 3560 | 5154 | ++ | ++ | ND |
| | 5031 | 3561 | 5155 | + | ++ | ND |
| | | 3562 | 5156 | ++ | +++ | ND |
| ATP5B | 5032; | 3563 | 5157 | ++ | +++ | ND |
| | 5033 | 3564 | 5158 | ++ | +++ | ND |
| | | 3565 | 5159 | ++ | +++ | ND |
| SLC25A5 | 5036; | 3572 | 5166 | + | ++ | ND |
| | 5037 | 3573 | 5167 | ++ | +++ | ND |
| | | 3574 | 5168 | ++ | +++ | ND |
| SLC25A6 | 5038; | 3575 | 5169 | +++ | +++ | ND |
| | 5039 | 3576 | 5170 | +++ | +++ | ND |
| | | 3577 | 5171 | ++ | +++ | ND |
| UQCRC2 | 5040; | 3578 | 5172 | + | ++ | ND |
| | 5041 | 3579 | 5173 | ++ | +++ | ND |
| | | 3580 | 5174 | ++ | ++ | ND |
| RRM2 | 4883; | 5306 | 5303 | ++ | + | ND |
| | 4884 | 5307 | 5304 | + | ++ | ND |
| | | 5308 | 5305 | ++ | ++ | ND |
| RRM2B | 5042; | 3584 | 5175 | ++ | ++ | ND |
| | 5043 | 3585 | 5176 | ++ | ++ | ND |
| | | 3586 | 5177 | ++ | +++ | ND |
| CTPS | 5044; | 3592 | 5178 | − | + | ND |
| | 5045 | 3593 | 5179 | − | + | ND |
| | | 3594 | 5180 | − | + | ND |
| IMPDH2 | 5046; | 3595 | 5181 | + | ++ | ND |
| | 5047 | 3596 | 5182 | + | ++ | ND |
| | | 3597 | 5183 | + | ++ | ND |

−: <50% mRNA knockdown
+: 50-70% mRNA knockdown
++: 70-90% mRNA knockdown
+++: >90% mRNA knockdown
ND: Not determined

TABLE 13
Effect of siRNA-mediated RNAi on level of mRNA on transporter gene
target transcript in A549 cells

| | RT-PCR | Antisense | | | | |
|---|---|---|---|---|---|---|
| | primers | DNA | Antisense | | | |
| | SEQ ID | SEQ ID | RNA | | | |
| Target gene | NO: | NO: | SEQ ID NO: | 0.1 nM | 1 nM | 10 nM |
| SLC2A1; GLUT1 | 5048; 5049 | 4892 | 5187 | − | − | ND |
| | | 4893 | 5188 | − | − | ND |
| | | 4894 | 5189 | − | ++ | ND |
| SLC2A5; GLUT5 | 5050; 5051 | 4895 | 5190 | ++ | ++ | ND |
| | | 4896 | 5191 | − | − | ND |
| | | 4897 | 5192 | +++ | +++ | ND |
| SLC2A12; GLUT12 | 5052; 5053 | 4898 | 5193 | +++ | +++ | ND |
| | | 4899 | 5194 | ++ | ++ | ND |
| | | 4900 | 5195 | ++ | ++ | ND |
| SLC29A1; ENT1 | 5054; 5055 | 4931 | 5226 | ++ | ++ | +++ |
| | | 4932 | 5227 | ++ | ++ | ++ |
| | | 4933 | 5228 | − | ++ | ++ |
| SLC29A2; ENT2 | 5056; 5057 | 4934 | 5229 | + | − | ND |
| | | 4935 | 5230 | + | + | ND |
| | | 4936 | 5231 | − | − | ND |
| SLC1A5; ASCT2 | 5058; 5059 | 4889 | 5184 | ++ | ++ | ND |
| | | 4890 | 5185 | ++ | ++ | ND |
| | | 4891 | 5186 | ++ | ++ | ND |
| SLC3A2; 4Fhc | 5060; 5061 | 4907 | 5202 | − | − | ND |
| | | 4908 | 5203 | − | + | ND |
| | | 4909 | 5204 | + | ++ | ND |
| SLC7A5; LAT1 | 5062; 5063 | 4901 | 5196 | ++ | ++ | ND |
| | | 4902 | 5197 | ++ | ++ | ND |
| | | 4903 | 5198 | ++ | +++ | ND |
| SLC7A11; xCT | 5064; 5065 | 4904 | 5199 | + | ++ | ND |
| | | 4905 | 5200 | ++ | ++ | ND |
| | | 4906 | 5201 | + | ++ | ND |
| SLC5A6; SMVT1 | 5066; 5067 | 4910 | 5205 | − | + | ND |
| | | 4911 | 5206 | ++ | ++ | ND |
| | | 4912 | 5207 | + | + | ND |
| SLC19A2; ThTr1 | 5068; 5069 | 4913 | 5208 | ++ | ++ | ND |
| | | 4914 | 5209 | ++ | +++ | ND |
| | | 4915 | 5210 | ++ | ++ | ND |
| SLC23A2; SVCT2 | 5070; 5071 | 4916 | 5211 | ++ | ++ | ND |
| | | 4917 | 5212 | ++ | ++ | ND |
| | | 4918 | 5213 | ++ | ++ | ND |
| SLC25A1; CIC | 5072; 5073 | 4925 | 5220 | ++ | ++ | ND |
| | | 4926 | 5221 | ++ | ++ | ND |
| | | 4927 | 5222 | ++ | ++ | ND |
| SLC25A10; DIC | 5074; 5075 | 4928 | 5223 | ++ | ++ | ND |
| | | 4929 | 5224 | − | + | ND |
| | | 4930 | 5225 | ++ | ++ | ND |
| SLC9A1; NHE1 | 5076; 5077 | 4919 | 5214 | ++ | ++ | ND |
| | | 4920 | 5215 | ++ | ++ | ND |
| | | 4921 | 5216 | ++ | ++ | ND |
| SLC16A1; MCT1 | 5078; 5079 | 4922 | 5217 | + | ++ | +++ |
| | | 4923 | 5218 | + | ++ | +++ |
| | | 4924 | 5219 | + | ++ | ++ |
| ATP2B1; PMCA1 | 5080; 5081 | 4937 | 5232 | ++ | ++ | ND |
| | | 4938 | 5233 | + | ++ | ND |
| | | 4939 | 5234 | + | ++ | ND |
| ATP2B4; PMCA4 | 5086; 5087 | 4946 | 5241 | ++ | ++ | ND |
| | | 4947 | 5242 | ++ | ++ | ND |
| | | 4948 | 5243 | − | ++ | ND |
| ATP6V1B2 | 5090; 5091 | 4952 | 5247 | − | + | ND |
| | | 4953 | 5248 | − | + | ND |
| | | 4954 | 5249 | + | + | ND |
| ATP6V1C1 | 5092; 5093 | 4955 | 5250 | + | ++ | ND |
| | | 4956 | 5251 | ++ | ++ | ND |
| | | 4957 | 5252 | ++ | ++ | ND |
| ATP6V1E1 | 5094; 5095 | 4958 | 5253 | + | ND | ND |
| | | 4959 | 5254 | − | ++ | ND |
| | | 4960 | 5255 | − | + | ND |
| ATP6V1F | 5096; 5097 | 4961 | 5256 | ++ | ++ | ND |
| | | 4962 | 5257 | − | + | ND |
| | | 4963 | 5258 | ++ | ++ | ND |
| ATP7A | 5098; 5099 | 4964 | 5259 | − | − | ND |
| | | 4965 | 5260 | − | − | ND |
| | | 4966 | 5261 | − | − | ND |
| ATP7B | 5100; 5101 | 4967 | 5262 | − | − | ND |
| | | 4968 | 5263 | − | ++ | ND |
| | | 4969 | 5264 | − | ++ | ND |
| ABCC4; MRP4 | 5102; 5103 | 4970 | 5265 | ++ | ++ | ND |
| | | 4971 | 5266 | − | ++ | ND |
| | | 4972 | 5267 | − | ++ | ND |
| ABCC5; MRP5 | 5104; 5105 | 4973 | 5268 | − | − | ND |
| | | 4974 | 5269 | − | + | ND |
| | | 4975 | 5270 | − | + | ND |
| ABCB1; MDR1 | 5106; 5107 | 4976 | 5271 | − | − | ND |
| | | 4977 | 5272 | − | − | ND |
| | | 4978 | 5273 | − | − | ND |
| | | 4979 | 5274 | ND | ND | ND |
| | | 4980 | 5275 | ND | ND | ND |

−: <50% mRNA knockdown
+: 50-70% mRNA knockdown
++: 70-90% mRNA knockdown
+++: >90% mRNA knockdown
ND: Not determined

### EXAMPLE 11

### Effect of siRNA-Mediated Inhibition of Gene Expression on Tumor Growth of Transfection of Cells Pre-Implantation

To determine the effect of siRNA-mediated knockdown of HK-II, CAD, IMPDH2, RRM2, SLC16A1 and SLC29A1 on tumor growth, A549 cells (human lung cancer cell line; ATCC No. CCL-185) were reverse-transfected prior to implantation in CD-1 nude mice as described above with siRNAs given in SEQ ID NO: 5307 (DNA), and SEQ ID NO: 5304 (RNA) for RRM2, SEQ ID NO: 4932 (DNA), and SEQ ID NO: 5227 (RNA) for ENT1, SEQ ID NO: 4813 (DNA), and SEQ ID NO: 5119 (RNA) for HK-II, SEQ ID NO: 4827 (DNA), and SEQ ID NO: 5133 (RNA) for CAD, SEQ ID NO: 4879 (DNA), and SEQ ID NO: 5182 (RNA) for IMPDH2 and SEQ ID NO: 4924 (DNA), and SEQ ID NO: 5219 (RNA) for SLC16A1, respectively. The siRNA given in SEQ ID NO: 4804 (DNA), and SEQ ID NO: 5301 (RNA) was used as control.

In two separate experiments, CD-1 mice were divided into five groups that received untransfected A549 cells, cells transfected with control siRNA or three different siRNAs (as listed above), respectively. Cells were transfected in vitro using the method as described in Example 8, and 24 h after transfection, s.c. inoculated into mice. Tumor growth was measured 3 times per week. In the first experiment, mice received cells transfected with siRNA targeting RRM2, HK-II or SLC29A1 and were monitored for 33 days. In the second experiment, mice received cells transfected with siRNA targeting CAD, IMPDH2 or SLC16A1, and were monitored for 26 days. Preliminary data showed no significant difference in tumor growth rate between the two control groups in each experiment. Thirty-three days after the cell inoculation, the average tumor size resulting from RRM2 siRNA inoculation (100.1±116.6 mm³) was significantly smaller than those of the tumors resulting from inoculation with untransfected cells (164.8±18.6 mm³; P<0.01) and the control siRNA transfectants (172.3±36.1 mm³; P<0.01). Similarly, preliminary data indicated tumor growth rate was reduced for the groups receiving cells transfected with siRNA targeting CAD and SLC16A1. Twenty-six days after cell inoculation, the tumor volume for mice with cells transfected with siRNA targeting CAD or SLC16A1 was 97.2±7.3 mm³ or 110.1±28.1 mm³, respectively. These tumor masses were significantly smaller than those resulting from untransfected cells (133.9±21.1 mm³, P<0.01 for CAD and P<0.05 for SLC16A1) and the control siRNA-treated cells (154.3±23.7 mm³, P<0.01 for CAD and P<0.05 for SLC16A1). No tumor growth inhibition was seen in mice that received cells transfected with siRNA targeting HK-II, SLC29A1 or IMPDH2 as compared to the control groups.

### EXAMPLE 12

### siRNA-Mediated Inhibition of YBX1 Gene Expression

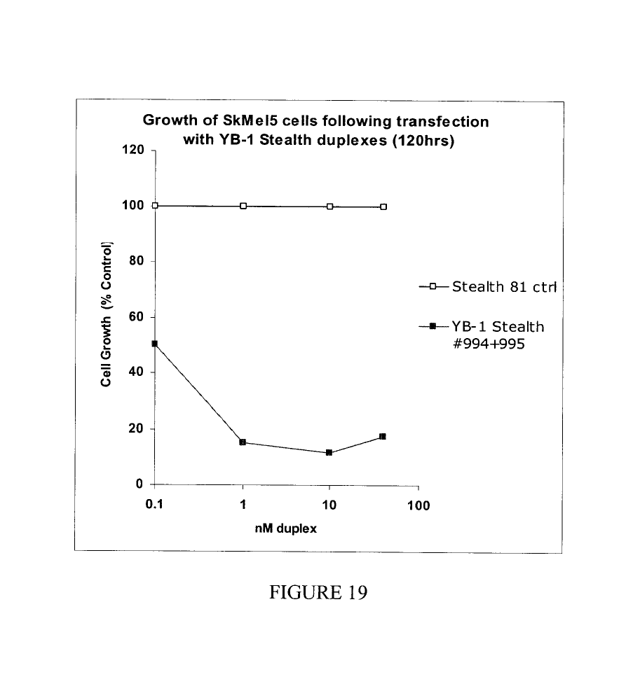

siRNA-mediated inhibition of cancer cell growth using siRNA targeting the YBX1 gene (SEQ ID NO: 92) was determined as follows. SK-MeI-5 cells were cultured in RPMI 1640 medium with 10% v/v heat-inactivated fetal calf serum. Cells were cultured as described above and reverse transfected with 0.1, 1.0, 10 and 40 nM (final concentration) of a pool of YBX1-targeting siRNAs (SEQ ID NO: 4982 and 4983 (DNA), and SEQ ID NO: 5277 and 5278 (RNA); referred to as 994 and 995 in FIG. 19) or a non-specific control (SEQ ID NO: 4804 (DNA), and SEQ ID NO: 5301 (RNA); referred to as Stealth 81 ctrl in FIG. 19), and cell growth determined after 120 h by measuring DNA fluorescence with a Wallac Victor² plate reader (Turku, Finland) set at an excitation frequency of 485 nm and measuring emission at 535 nm. Growth inhibition by the siRNAs is shown in FIG. 19. Growth inhibition by the siRNA pool was standardized against the inhibition obtained with the control siRNAs.

siRNA-mediated RNAi was also determined on a panel of different cell lines. A549, SK-MeI-5, wt HCT-116, HCT-116−/−p53 and MCF-7 cells were transfected with 10 nM siRNAs targeting YBX-1 and growth inhibition determined as described above. The YBX1 siRNAs are given in SEQ ID NO: 4981 to 4985 (DNA) and SEQ ID NO: 5276-5280 (RNA). A non-specific siRNA (SEQ ID NO: 4804 (DNA) and SEQ ID NO: 5301 (RNA)) was used as control. Growth inhibition by the siRNAs, given in Table 14, was standardized against the inhibition obtained with the control siRNA.

TABLE 14
Effect of YBX-1 siRNA-mediated RNAi on growth of a panel of cancer
cell lines

| Antisense | Antisense | | SK- | | HCT-116 | |
|---|---|---|---|---|---|---|
| DNA SEQ | RNA SEQ | A549 | Mel- | wt HCT- | −/−p53 | MCF7 |
| ID NO: | ID NO: | cells | 5 cells | 116 cells | cells | cells |
| 4981 | 5276 | + | ++ | ND | ND | ND |
| 4982 | 5277 | +++ | +++ | +++ | +++ | ++ |
| 4983 | 5278 | + | ++ | ND | ND | ND |
| 4984 | 5279 | ++** | +++ | +* | +* | ND |
| 4985 | 5280 | +** | ++ | −* | −* | ND |

−: No inhibition
+: At least 20% inhibition
++: At least 50% inhibition
+++: At least 70% inhibition
**by 5 nM siRNA
*by 1 nM siRNA
ND: Not done

Effect of siRNA Knockdown on YBX1 mRNA Level

A549 cells were transfected with 0.1, 1.0 and 10 nM siRNA (given in SEQ ID NO: 4981 to 4985 (DNA), and SEQ ID NO: 5276-5280 (RNA)) and the relative mRNA levels quantified 24 h post-transfection as described in Example 8. Reduction in mRNA levels achieved is given in Table 15.

TABLE 15
YBX1-specific reduction of mRNA levels in A549 cells

| | RT-PCR | Antisense | Antisense | | | |
|---|---|---|---|---|---|---|
| Target | primers SEQ | DNA SEQ | RNA SEQ | | | |
| gene | ID NO: | ID NO | ID NO | 0.1 nM | 1 nM | 10 nM |
| YBX1 | 5108; 5109 | 4981 | 5276 | ND | ND | ++ |
| | | 4982 | 5277 | ND | +++ | +++ |
| | | 4983 | 5278 | ND | ND | ++ |
| | | 4984 | 5279 | +++ | +++ | +++ |
| | | 4985 | 5280 | + | ++ | ++ |

+: At least 20% inhibition
++: At least 50% inhibition
+++: At least 70% inhibition
ND: Not done

Effect of siRNA Knockdown on YBX1 Protein Levels

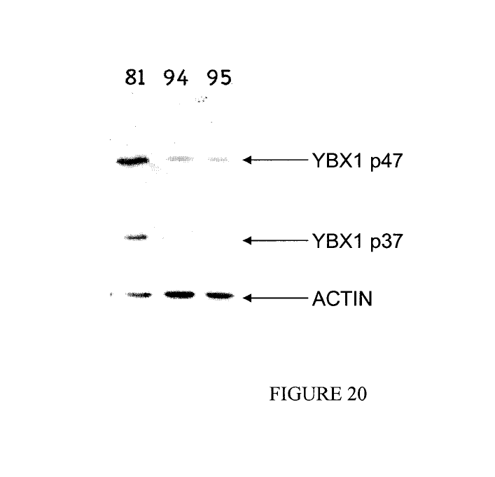

Cell lysates were prepared from A549 cells 24 h post-transfection with 10 nM YBX1-specific siRNA (SEQ ID NO: 4982 (DNA), and SEQ ID NO: 5277 (RNA) was labeled 94; SEQ ID NO: 4983 (DNA), and SEQ ID NO: 5278 (RNA) was labeled 95) or control siRNA (SEQ ID NO: 4804 (DNA), and SEQ ID NO: 5301 (RNA); labeled 81). Following Western blotting onto Immobilon-P PVDF filer (Millipore, Bedford Mass.), membranes were probed with a goat anti-human polyclonal antibody specific for YBX1 (AbCam plc, Cambridge, UK), at a concentration of 0.4 mg/ml. HRP-conjugated donkey anti-goat IgG (80 mg/ml; Santa Cruz) was used as a secondary antibody, and signal was detected with an ECL Plus Western blotting Detection System (GE Healthcare, UK) using a Typhoon Scanner (Molecular Dynamics, GE Healthcare, UK). FIG. 20 shows siRNA-mediated reduction in YBX1 (p47) and p37 protein levels in the cells compared with the control siRNA.

Effect siRNA-Mediated Inhibition of YBX1 Gene Expression in A549 SK-MeI-5 and HCT-116−/−p53 Cells Grown in Hypoxic Versus Normoxic Conditions

A549, SK-MeI-5 and HCT-116−/−p53 cells were cultured in RPMI 1640 medium with 10% v/v heat-inactivated fetal calf serum. In a 96-well plate, 1,500 cells were reverse transfected using Lipofectamine™ RNAiMAX transfection reagent with 1.0 nM (final concentration) of YBX1-targeting siRNAs labeled 994, 856 and 931 (SEQ ID NO: 4982, 4984 and 4985 (DNA), and SEQ ID NO: 5277, 5279 and 5280 (RNA), respectively) or a non-specific control (81c; SEQ ID NO: 4804 (DNA), and SEQ ID NO: 5301 (RNA)). After incubating the cells for 24 h, the transfection media were replaced with 150 μl fresh cRPMI supplemented with 10% FBS, 0.05 mM 2-Mercaptoethanol, and containing PSG (160 μM penicillin, 70 μM streptomycin, 20 μM L-glutamine). For the hypoxic treatment, plates were placed into an anoxic chamber, flushed with 5% CO₂/95% N at 20 litres/min for 4 minutes (80 litres total), sealed and replaced into an incubator at 37° C., 5% CO₂, until harvesting. The flush was repeated once more after 24 h to remove any residual O₂. For the normoxic treatment, plates were incubated in a humidified box in an incubator at 37° C., 5% CO₂ until harvesting.

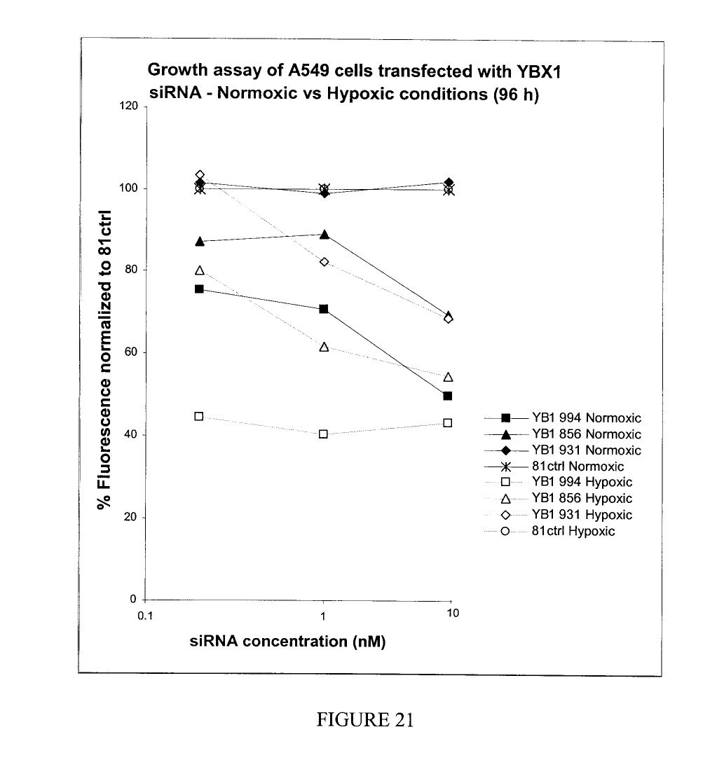

At 120 h following reverse transfection (96 h in hypoxic conditions), cells were harvested and frozen at −80° C. overnight. Growth inhibition was measured using Syber Green as described above. In FIG. 21, the growth inhibition in A549 cells grown under normoxic and hypoxic conditions by the YBX1-targeting siRNAs is shown.

Effect of Co-Transfection with p53-Targeting siRNA on YBX1 siRNA-Mediated Growth Inhibition

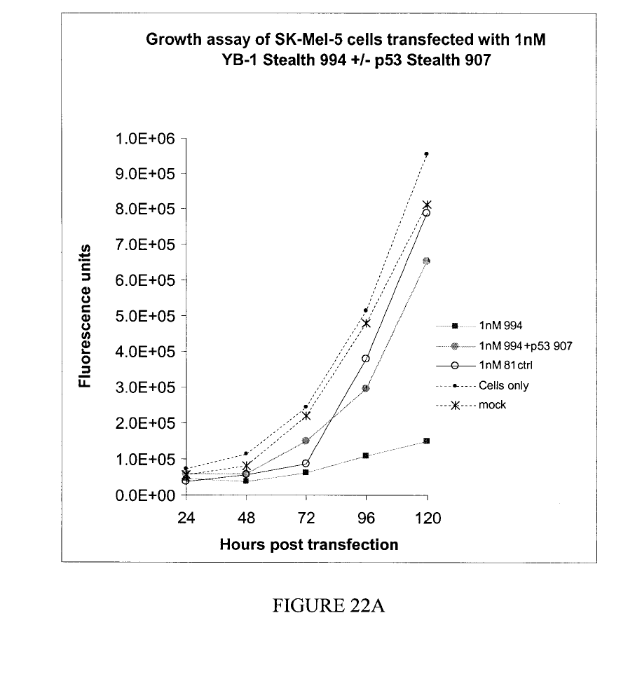

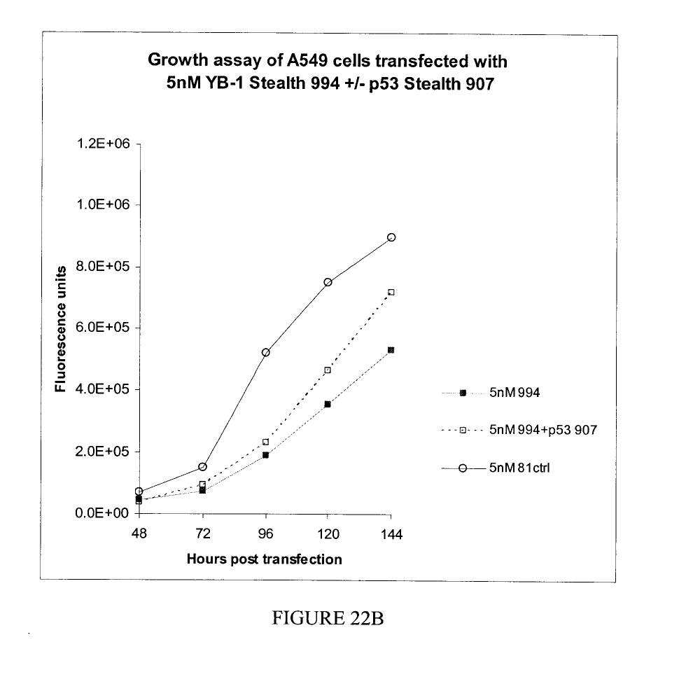

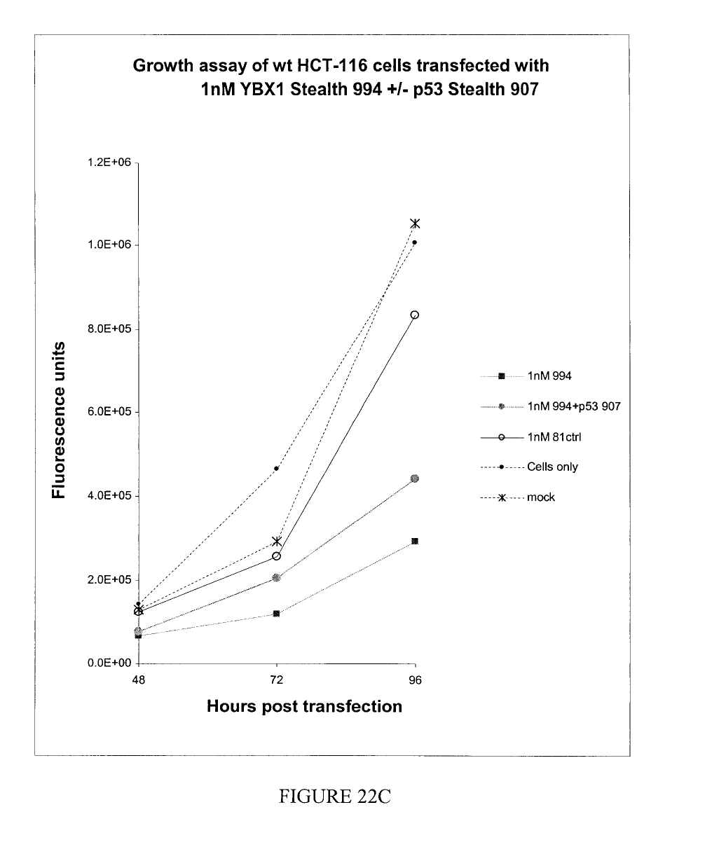

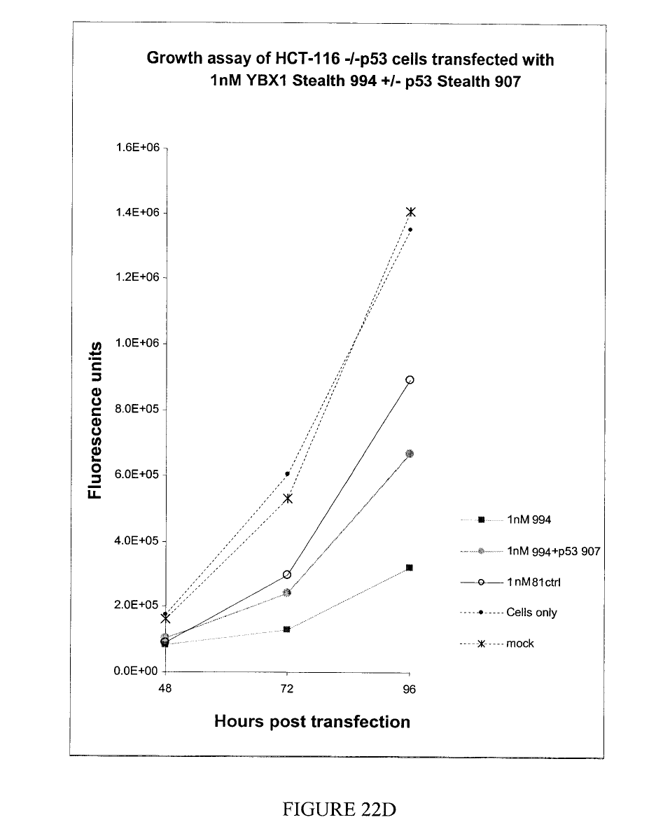

Transfection of cells (A549, SK-Mel-5, wt HCT-116, HCT-116−/−p53) and determination of growth inhibition were performed as described above using Lipofectamine RNAiMAX, and a YBX1-specific siRNA (given in SEQ ID NO: 4982 (DNA), and SEQ ID NO: 5277 (RNA); 1 nM siRNA for SK-MeI-5 cells and 5 nM siRNA (A549 cells), plus and minus equimolar levels of the p53-targeting Stealth™ siRNA given in SEQ ID NO: 5110 (DNA), and SEQ ID NO: 5299 (RNA). Controls included cells only, mock-transfected cells and a non-specific siRNA control (labeled 81 ctrl; SEQ ID NO: 4804 (DNA), and SEQ ID NO: 5301 (RNA)). Results showed that co-transfection with the p53 duplex completely ablated growth inhibition by the YBX1-specific siRNA in SK-MeI-5 cells (FIG. 22A), but in the A549 (FIG. 22B), wt HCT-116 (FIG. 22C) and HCT-116−/−p53 (FIG. 22D), only partial abrogation of growth inhibition was observed. This could reflect either that a greater concentration of duplex was required to significantly affect p53 levels in the cells used, or that growth inhibition occurred via a p53-independent mechanism.

### EXAMPLE 14

### Increased Drug Toxicity Following siRNA Transfection In Vitro

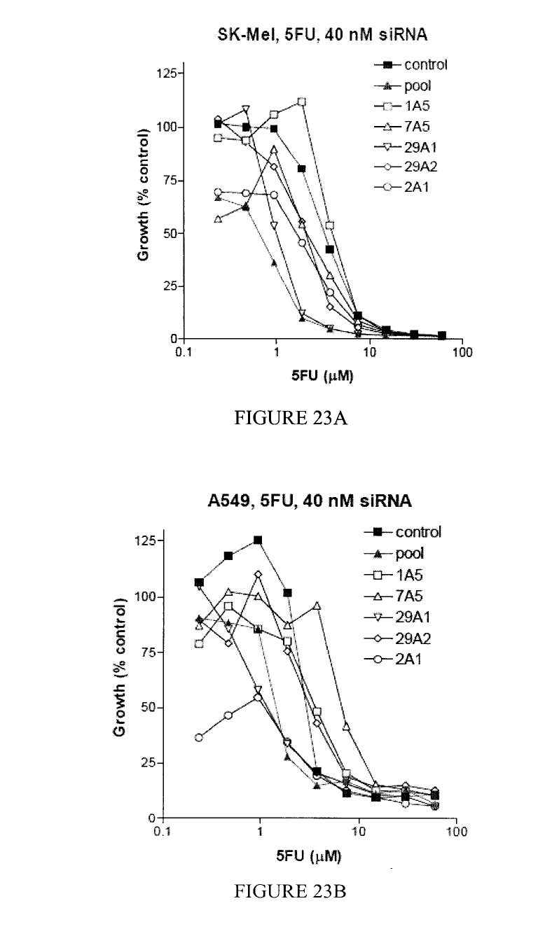

The effect of siRNA-mediated transporter knockdown on cancer cell sensitivity to chemotherapeutics was tested as follows. A549, SK-MeI-5 and HCT-15 cells (1.5×10³ cells) were transfected in triplicate in 96-well plates with a final concentration of 40 nM of a pool of three siRNAs targeting SLC1A5, SLC2A1, SLC7A5, SLC29A1, SLC29A2 (siRNA SEQ ID NO: as listed in Table 12), or control siRNA (SEQ ID NO: 4804 (DNA), and SEQ ID NO: 5301 (RNA)). Transfections contained three siRNAs against each individual target (1A5, 7A5, 29A1, 29A2, 2A1) or one siRNA against each of the five targets (pool). Lipofectamine 2000 (Invitrogen) was used as the transfection agent. At 24 h post-transfection, cells were exposed continuously to chemotherapeutics at the concentrations indicated for 72-96 h, at which point plates were harvested by removing medium and freezing at −80° C. The next day, plates were removed and DNA content in wells was quantified with the CyQuant™ cell proliferation kit (Invitrogen). As shown in FIGS. 23A and B, SK-MeI-5 and A549 cells transfected with siRNAs targeting glucose or nucleoside transporters exhibited increased sensitivity to 5FU compared with controls.

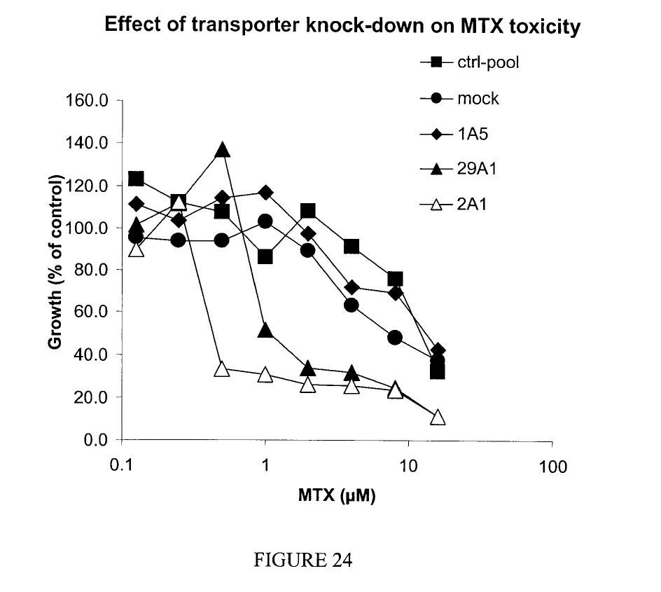

The results obtained for methotrexate are shown in FIG. 24. HCT-15 cells were transfected with 40 nM siRNA against the listed gene targets. Transfections contained three siRNAs against each individual target (SLC2A1 (2A1), SLC29A1 (29A1) and SLC1A5 (1A5)). A pool of control siRNA (SEQ ID NO: 4804 and 4805 (DNA), and SEQ ID NO: 5301 and 5302 (RNA)) was used. Following transfection, cells were exposed to methotrexate (MTX) at the concentration indicated for 4 h, after which medium was replaced and cells were grown for 96 h. DNA content in wells was measured using CyQuant. FIG. 24 shows that, when compared with mock transfected cells or transfection with control siRNA (filled circles and squares, respectively), cells transfected with siRNAs targeting SLC2A1 (open triangles) or SLC29A1 (filled triangles) were more sensitive to methotrexate treatment. The sensitivity of cells transfected with SLC1A5-specific siRNAs (filled diamonds) did not change.

### Effect of MDR1 Knockdown on Drug Resistance

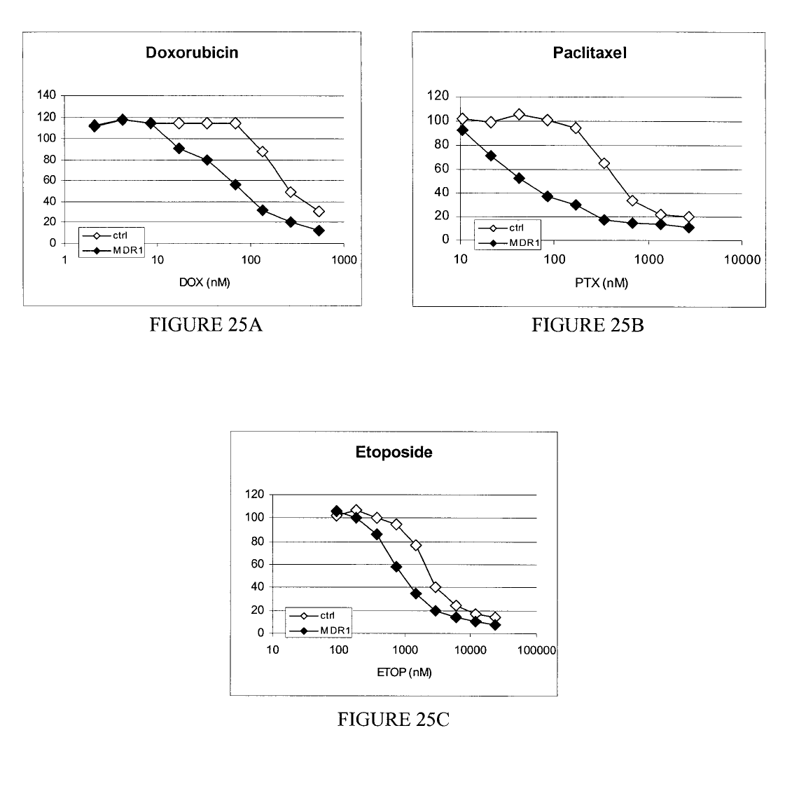

HCT-15 cells, which express high endogenous levels of MDR1, were reverse transfected with MDR1-specific (SEQ ID NO: 4976 (DNA), and SEQ ID NO: 5271 (RNA)) or control siRNAs (SEQ ID NO: 4804 (DNA), and SEQ ID NO: 5301 (RNA)) at a final concentration of 10 nM. Lipofectamine-siRNA complexes were made by adding 0.3 μl Lipofectamine RNAiMAX in 10 μl RPMI to an equivalent volume of medium containing siRNA, such that the final siRNA concentration would be 10 nM. This mixture was incubated at room temperature for 20 minutes to allow complexes to form, after which time 1,500 HCT-15 cells in 100 μl RPMI supplemented with 10% FCS were added to the wells. After incubation at 37° C. for 24 h, transfection medium was replaced with fresh medium containing a serial dilution of chemotherapeutic drug as indicated in FIG. 22, and cells were exposed continuously to drug for 72 h. Drugs used were Doxorubicin (Sigma, St. Louis Mo.), Paclitaxel (Invitrogen) and Etoposide (Sigma, St. Louis Mo.). At the conclusion of the assay, medium was removed and growth inhibition determined as described above. Cells transfected with the MDR1-targeting siRNA (FIG. 25; filled symbols) became more sensitive to the MDR1 substrates doxorubicin (FIG. 25A), paclitaxel (FIG. 25B), and etoposide (FIG. 25C), compared with control siRNA-transfected cells (FIG. 25A-C; open symbols).

### Effects of TYMS and DHFR Knockdown on Tumor Cell Growth and Sensitivity to 5FU

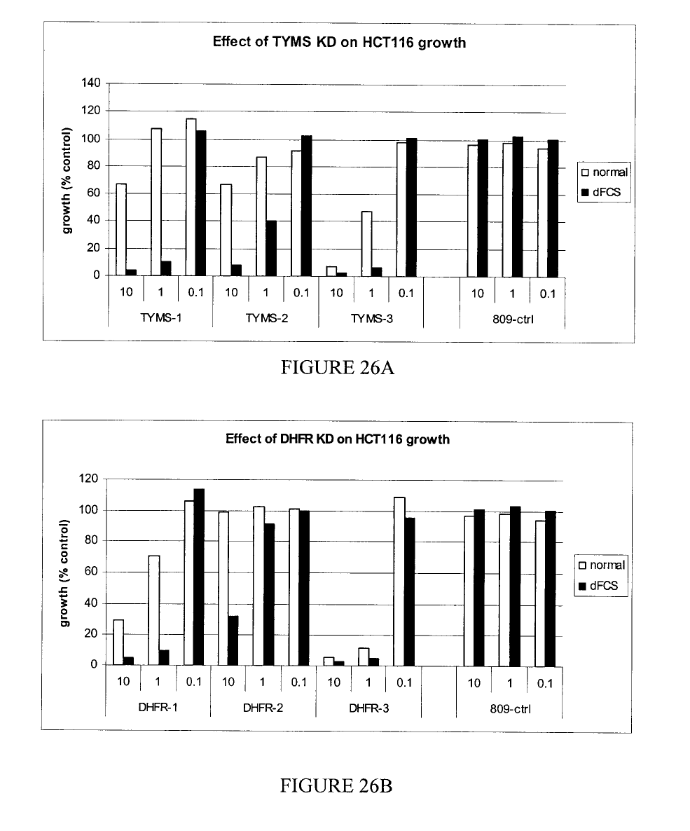

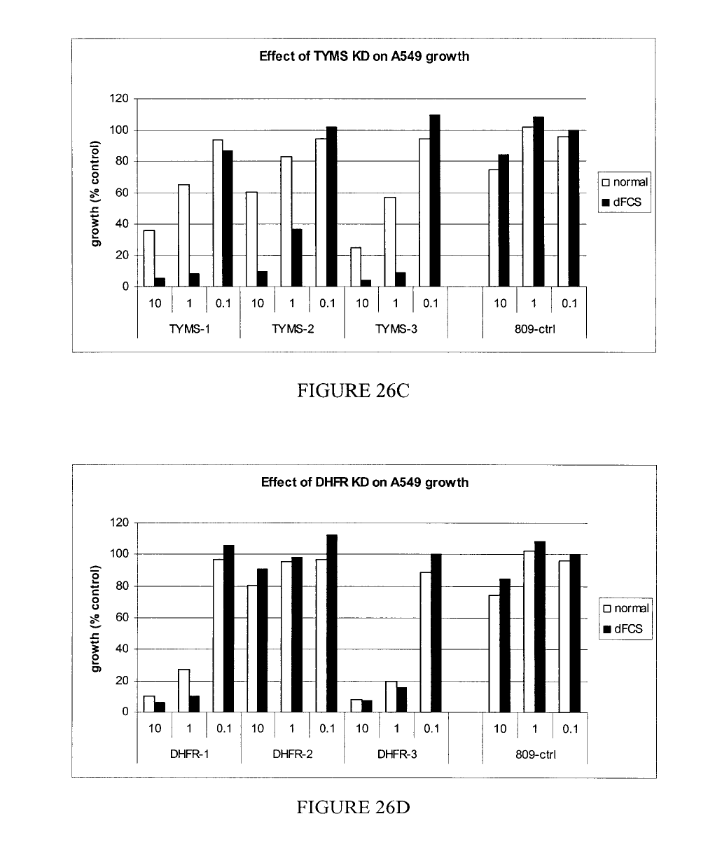

HCT-116 and A549 cells were transfected with 0.1, 1.0 and 10 nM siRNA targeting TYMS and DHFR (SEQ ID NO: 4833 to 4838 (DNA), and SEQ ID NO: 5139-5144 (RNA)) using Lipofectamine™ RNAiMAX and incubated for 96 h, in either normal fetal calf serum or serum dialyzed to remove low molecular weight molecules (cut-off 10 kDa). Growth of cells was measured by measuring the total DNA content per well using the SYBR Green assay as described above, and values are given as the percentage of the averaged control values. FIG. 26 shows that knockdown of TYMS and DHFR by three independent siRNAs lead to an inhibition of both HCT-116 and A549 cell growth. Inhibition increased with increasing siRNA concentration, which was not observed with a non-specific control siRNA. This dose-dependent inhibition was potentiated when experiments were carried out in medium supplemented with dialyzed fetal calf serum. FIGS. 26A and B show the effects of knockdown on tumor cell growth and sensitivity to 5FU by TYMS and DHFR siRNA in the presence of normal or dialyzed fetal calf serum in HCT-116 cells, and FIGS. 26C and D show the results obtained in A549 cells.

### Effect of TYMS and DHFR Knockdown on 5FU Toxicity.

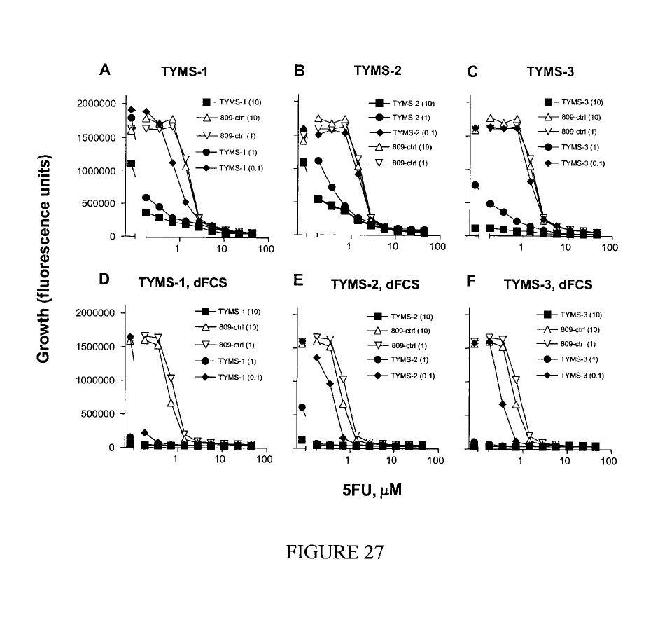

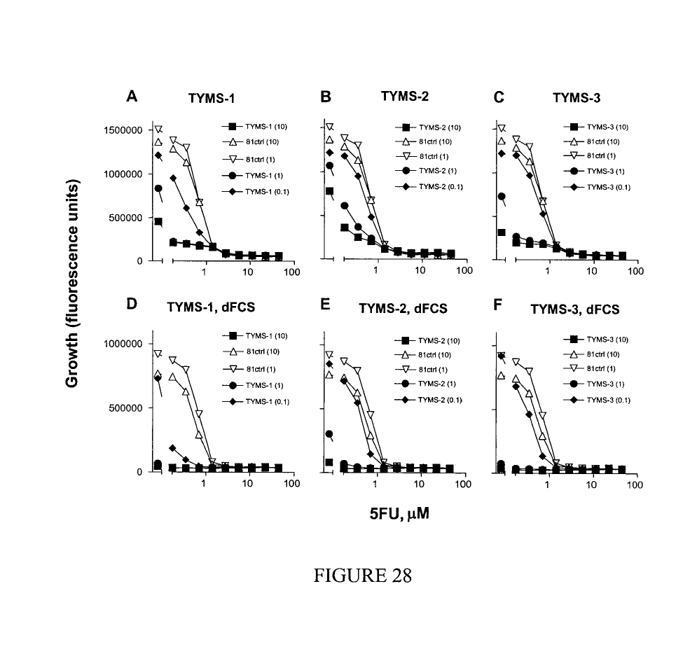

HCT-116 and A549 cells were transfected as described above with 0.1, 1.0 and 10 nM of three TYMS-specific siRNAs, labeled TYMS-1, TYMS-2 and TYM-3 (SEQ ID NO: 4833-4835 (DNA), and SEQ ID NO: 5139-5141 (RNA), or 1.0 and 10 nM control siRNAs (809-ctrl; SEQ ID NO: 4805 (DNA), and SEQ ID NO: 5302 (RNA) and 81ctrl; SEQ ID NO: 4804 (DNA), and SEQ ID NO: 5301 (RNA)). HCT-116 and A549 cells were also similarly transfected with 0.1, 1.0 and 10 nM of three DHFR-specific siRNAs, labeled DHFR-1, DHFR-2 and DHFR-3 (SEQ ID NO: 4836-4838 (DNA), and SEQ ID NO: 5142-5144 (RNA)), or control siRNA (SEQ ID NO: 4805 (DNA), and SEQ ID NO: 5302 (RNA)). Twenty four hours after transfection, cells were exposed to 5FU using a serial dilution starting at 44 μM, in medium containing either 10% normal FCS or 10% dialyzed FCS as described above. 5FU treatment was continuous for 3 days, at which time cell growth was analyzed by measuring DNA content per well using the SYBR Green assay outlined above. As seen in FIGS. 27 and 28, transfection with TYMS siRNA led to a dose-dependent increase in growth inhibition above that caused by the drug alone (control siRNA-transfected cells) in HCT-116 cells (FIGS. 27A-C), and A549 cells (FIGS. 28A-C). This combinatorial inhibitory effect was further increased when experiments were done in medium containing dialyzed FCS (FIGS. 27D-F; FIGS. 28D-F).

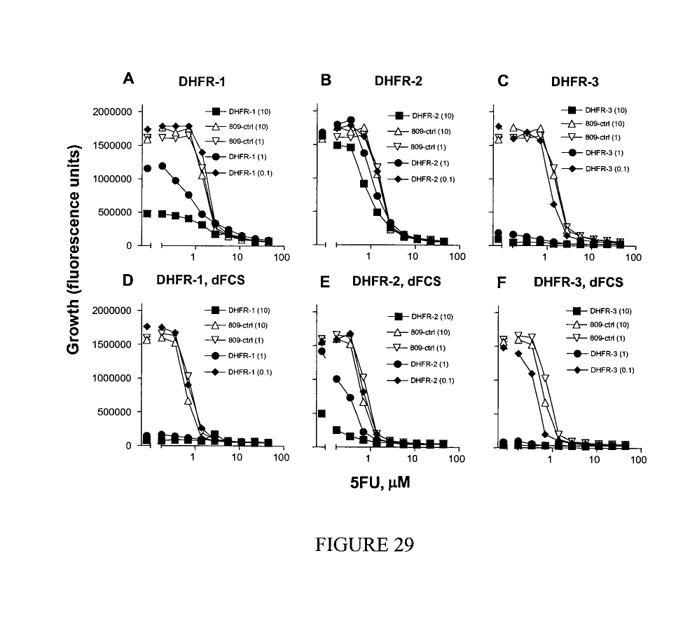

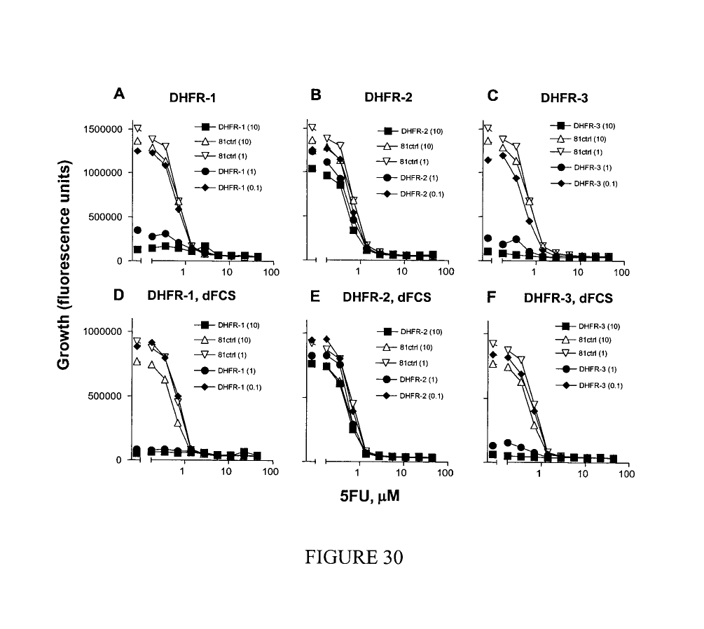

FIGS. 29 and 30 show transfection with DHFR siRNA similarly led to a dose-dependent increase in growth inhibition above that caused by the drug alone (control siRNA-transfected cells) in HCT-116 cells (FIGS. 29A-C), and A549 cells (FIGS. 30A-C). This combinatorial inhibitory effect was further increased when experiments were done in medium containing dialyzed FCS (FIGS. 29D-F; FIGS. 30D-F). The changes in the IC₅₀ value for 5FU in HCT-116 and A549 cells following TYMS knockdown by individual siRNAs are listed in Table 16 below.

TABLE 16
Toxicity of 5FU following TYMS knockdown

| | Antisense | | | |
|---|---|---|---|---|
| siRNA identifier | DNA | Antisense | IC₅₀ value (μM) | |
| and concentration | SEQ ID NO | RNA SEQ ID NO | HCT-116 | A549 |
| TYMS-1 (10 nM) | 4833 | 5139 | 0.09 | 0.14 |
| TYMS-1 (1 nM) | | | 0.09 | 0.11 |
| TYMS-1 (0.1 nM) | | | 0.10 | 0.34 |
| TYMS-2 (10 nM) | 4834 | 5140 | 0.10 | 0.15 |
| TYMS-2 (1 nM) | | | 0.09 | 0.21 |
| TYMS-2 (0.1 nM) | | | 0.41 | 0.59 |
| TYMS-3 (10 nM) | 4835 | 5141 | 0.11 | 0.20 |
| TYMS-3 (1 nM) | | | 0.19 | 0.13 |
| TYMS-3 (0.1 nM) | | | 0.32 | 0.61 |
| 809-ctrl (10 nM) | 4805 | 5302 | 0.60 | 0.64 |
| 809-ctrl (1 nM) | | | 0.60 | 0.67 |
| 809-ctrl (0.1 nM) | | | 0.65 | 0.82 |

### EXAMPLE 15

### siRNA-Mediated Silencing of Expression in Mice

Human tumor cells can be transplanted into nude mice for use as an animal model of human tumor growth. Tumor growth can be measured in vivo at both the microscopic and macroscopic levels using histopathology and regular measurements of tumor size respectively. Tumor cells can be transfected with siRNA duplexes in vitro, prior to transplantation into host mice; or tumor targeting siRNA duplexes can be injected into the mice (directly into the tumor or systemically) once the tumor cells are established at the injection site (i.e. forming a tumor). Tumor growth over time is measured using calipers; and via histopathology at the end of the study.

The ability of the inventive compositions to inhibit tumor growth is examined in a mouse model of human tumor development. The extent of tumor growth is reflected in the tumor size over time.

Nude mice are given human tumor cells by the subcutaneous route at time 0. The injection site is monitored to establish the time of palpable tumor development and ongoing measurements of tumor size are made using calipers once the tumor is palpable. The inventive compositions are administered either directly to the tumor cells (using standard in vitro transfection procedures) prior to transplantation of these cells into host mice; injected directly into the tumor itself, once it is established in the host mice; or intravenously at various times during tumor development. Tumor development is determined and compared to control mice.

There are a number of model systems using human tumor cells as a means of identifying novel anti-cancer drugs. A reduction in tumor size upon treatment with the inventive compositions indicates that the compositions may be effectively employed in treating cancer.

### EXAMPLE 16

### Uptake of siRNA into Tumor Cells In Vivo

An Alexa 555-labeled control siRNA was used to determine the cellular uptake of siRNA in tumor tissues when given via intratumoral (i.t.) injection. A549 cells (6−8×10⁶ cells) were s.c. inoculated into CD-1 nude mice. Once the A549 tumors reached a size of approximately 100 mm³, mice were divided into two groups of four mice each. The Alexa 555-labeled Stealth™ siRNA (SEQ ID NO: 5111 (DNA), and SEQ ID NO: 5300 (RNA); Invitrogen Corp.) at a dose of 25 μg (in 50 μl) was injected into tumors of one group. 50 μl PBS was injected into tumors of a negative control group. Tumors were harvested at 6 and 24 h after injection and placed in a labeled cryomold (Tissue-TeK® Crymold®; ProSciTech, Thuringowa, Queensland, Australia) and covered with OCT tissue freezing medium (O.C.T. Compound, ProSciTech). The mold was gently submerged in liquid nitrogen for approximately one minute until OCT turned white and opaque. Tissues were stored at minus 80° C. until sections were cut. Tissues were allowed to reach −20° C. before processing in a Leica Cryostat (Leica Microsystems, Inc., Bannockburn Ill.) at 10 microns. Sections were picked up on Super Frost Plus slides (Esco, Biolab Scientific, New Zealand). After air-drying, sections were fixed in freshly prepared 2% paraformaldehyde solution for 2 minutes at room temperature and then rinsed in PBS to remove the fixative. The slides were mounted in Prolong Gold antifade reagent with DAPI (Molecular Probes®, Invitrogen Corp.), covered with cover slips and sealed to the slides with clear nail polish. The slides were stored at 4° C. in the dark. Fluorescence in the tumor samples was visualized using a Zeiss Axioskop Microscope (Carl Zeiss microimaging, Inc., Thornwood N.Y.) and Leica SP-2 Confocal Microscope (Leica Microsystems, Inc.). Autofluorescence in tumor tissues was very low in all filters examined (green, red and UV). At 6 h after the injection of Alexa 555-labeled siRNA, red punctuations were seen in some tumor cells, indicative of uptake of siRNA. Overlayed images captured from red and UV filters that revealed nuclei from DAPI staining, showed that the red punctuated emissions were located in the cytoplasm around the periphery of the nuclei. It is widely accepted that this pattern of peri-nuclear localization of siRNA is strongly correlated to siRNA knockdown, i.e. RNAi efficiency (Chiu et al., Chem. Biol. 11:1165-1175, 2004; Berezhna et al., Proc. Natl. Acad. Sci. USA 103:7682-7687, 2006). The fluorescent emission from the labeled siRNA was also observed in the samples taken 24 h after injection, although in slightly lower number of cells. The observed emission pattern was absent in tumors injected with PBS.

### EXAMPLE 17

### Suppression of RNAi

The therapeutic use of siRNA to knockdown target gene production in cancer cells in humans and non-human animals may require rapid reversal. Suppressor proteins from plant viruses are capable of reversing silencing in plant tissues where it is established, and preventing initiation of silencing in new tissues. Plant virus genes encoding suppressor proteins include HC-Pro (Tobacco etch virus), P25 (Potato virus X), 2b (Cucumber mosaic virus), Turnip crinkle virus coat protein, and p19 (Cymbidium ringspot virus).

Some plant virus silencing suppressor proteins are functional when expressed in cultured Drosophila cells (Reavy and MacFarlane. Scottish Crop Research Institute (SCRI) Annual Report 1000/2001, pp. 120-123). The B2 gene of the flock house virus (FHV), a nodavirus that infects vertebrate and invertebrate hosts, initiates and is a target of RNA silencing in plants and Drosophila cells (Li et al., Science 196:1319-21, 2002). Vaccinia virus and human influenza A, B and C viruses each encode viral suppressors (E3L and NSI) which bind dsRNA and inhibit the mammalian IFN-regulated innate antiviral response (Li et al., Proc. Natl. Acad. Sci. USA 101:1350-1355, 2004).

The effectiveness of these viral suppressors of RNAi may be evaluated as described above in Examples 6 and 7.

### EXAMPLE 18

### Screening of Candidate Therapeutic siRNA Molecules

a) In vitro Screening

siRNA target sequences may be screened in a high-throughput fashion by comparing the gene silencing effect of the target-specific siRNAs with the induction of cell death by known cancer therapeutic drugs in plate-based in vitro assays or using flow cytometry. Potential drugs that may be suitably employed in such methods include, but are not limited to, those provided in Table 17 below. The comparison includes the gene-silencing effect in normal and tumor cells, but also includes a comparison of normal and drug-resistant tumor cells. A comparative assay further includes the gene-silencing effect under aerobic conditions with hypoxia-induced genes turned on or off.

The ability of siRNAs to downregulate their target sequences may be tested in a model system by co-transfection of a cDNA encoding the target message and the siRNA to be tested as detailed above. Such systems comprise an easily transfectable cell line, e.g., HEK293. The activity of selected siRNA sequences against endogenously expressed target genes may be tested by transfecting primary B cells, mast cells, or cell lines derived from these cell types in vitro using commercially available transfection reagents (for example, Lipofectamine™2000, Invitrogen), electroporation (BTX ECM600), lipid-based complexes without targeting, or more specifically with transferrin receptor- and CD19-specific antibody-liposome complexes containing siRNA.

A screening sequence could be: (a) siRNA to silence a gene expressed in normal but not tumor cells; (b) siRNA directed to genes expressed in both normal and tumor targets but under the control of a promoter expressed only in tumor cells; (c) siRNA directed to a gene target which has resulted in drug resistance; and (d) siRNA to a hypoxic gene target expressed in tumor cells growing aerobically.

TABLE 17
Cancer Drugs Involved in Inhibition of in vivo Metabolic Pathways

| | Main | RNAi | | Related |
|---|---|---|---|---|
| Drug | action | Target | Information | Cancer(s) |
| Xeloda ® | Inhibition of | Thymidylate | Enzymatically converted to | Metastatic |
| (capecitabine) | DNA | synthetase | 5-fluorouracil (5-FU) in vivo | breast cancer; |
| | synthesis | | | metastatic |
| | | | | colorectal |
| | | | | carcinoma; |
| | | | | metastatic |
| | | | | carcinoma of |
| | | | | the ovary; non- |
| | | | | small cell lung |
| | | | | cancer |
| Fludara ® | Inhibition of | DNA | Fludarabine phosphate is | B-cell |
| (Fludarabine | DNA | polymerase | dephosphorylated to 2- | lymphocytic |
| phosphate) | synthesis | alpha, ribonucleotide | fluoro-ara-A and then | leukemia (CLL) |
| | | reductase or | phosphorylated | |
| | | DNA | intracellularly by | |
| | | primase | deoxycytidine kinase to the | |
| | | | active triphosphate, 2-fluoro- | |
| | | | ara-ATP. This metabolite | |
| | | | appears to act by inhibiting | |
| | | | DNA polymerase alpha, | |
| | | | ribonucleotide reductase and | |
| | | | DNA primase, thus | |
| | | | inhibiting DNA synthesis. | |
| Methotrexate | Inhibits | Dihydrofolic | Inhibits dihydrofolic acid | Breast cancer, |
| | DNA | acid | reductase. Dihydrofolates | epidermoid |
| | synthesis | reductase, | must be reduced to | cancers of the |
| | | thymidylate | tetrahydrofolates by this | head and neck, |
| | | synthetase. | enzyme before they can be | advanced |
| | | | utilized as carriers of one- | mycosis |
| | | | carbon groups in the | fungoides, and |
| | | | synthesis of nucleotides and | lung cancer, |
| | | | thymidylate. Methotrexate | particularly |
| | | | interferes with DNA | squamous cell |
| | | | synthesis, repair, and cellular | and small cell |
| | | | replication. | types stage; |
| | | | | non-Hodgkin's |
| | | | | lymphomas; |
| | | | | non-metastatic |
| | | | | osteosarcoma |
| | | | | meningeal |
| | | | | leukemia |
| Exemestane | Inhibits | Aromatase | An irreversible, steroidal | Breast cancer |
| (Aromasin ®) | aromatase | | aromatase inactivator, | |
| | | | structurally related to the | |
| | | | natural substrate | |
| | | | androstenedione. It acts as a | |
| | | | false substrate for the | |
| | | | aromatase enzyme, and is | |
| | | | processed to an intermediate | |
| | | | that binds irreversibly to the | |
| | | | active site of the enzyme | |
| | | | causing its inactivation, an | |
| | | | effect also known as “suicide | |
| | | | inhibition.” | |
| Aloprim ™ | DNA | Xanthine | Allopurinol is a structural | Leukemia, |
| (allopurinol) | synthesis | oxidase | analogue of the natural | lymphoma, and |
| | | | purine base, hypoxanthine. | solid tumor |
| | | | It is an inhibitor of xanthine | malignancies |
| | | | oxidase, the enzyme | |
| | | | responsible for the | |
| | | | conversion of hypoxanthine | |
| | | | to xanthine and of xanthine | |
| | | | to uric acid, the end product | |
| | | | of purine metabolism in man. | |
| Aromasin ®; | Inhibits | Aromatase | The principal source of | Breast cancer |
| Arimidex ® | aromatase | | circulating estrogens in | |
| (anastrozole); | | | postmenopausal women is | |
| Teslac; | | | from conversion of adrenal | |
| Femara | | | and ovarian androgens | |
| (letrozole); | | | (androstene-dione and | |
| | | | testosterone) to estrogens | |
| | | | (estrone and estradiol) by the | |
| | | | aromatase enzyme in | |
| | | | peripheral tissues. Estrogen | |
| | | | deprivation through | |
| | | | aromatase inhibition is an | |
| | | | effective and selective | |
| | | | treatment for some | |
| | | | postmenopausal patients with | |
| | | | hormone-dependent breast | |
| | | | cancer. | |
| FUDR | Inhibits | Thymidylate | FUdR is rapidly catabolized | Gastrointestinal |
| (fluorouridine) | DNA | synthetase | to 5-fluoro-uracil and | adenocarcinoma |
| | synthesis | (TYMS) | interferes with the synthesis | |
| | | | of DNA and to a lesser | |
| | | | extent inhibit the formation | |
| | | | of RNA. When FUDR is | |
| | | | given by continuous intra- | |
| | | | arterial infusion its direct | |
| | | | anabolism to FUDR- | |
| | | | monophosphate is enhanced, | |
| | | | thus increasing the inhibition | |
| | | | of DNA. | |
| Adrucil ® (5- | Inhibits | TYMS | Fluorouracil interferes with | Carcinoma of |
| Fluorouracil) | DNA | | the synthesis of DNA and to | the colon, |
| | synthesis | | a lesser extent inhibits the | rectum, breast, |
| | | | formation of RNA. The | stomach and |
| | | | effects of DNA and RNA | pancreas. |
| | | | deprivation are most marked | |
| | | | in rapidly growing cells | |
| | | | which take up fluorouracil at | |
| | | | a more rapid rate. | |
| Faslodex ® | Estrogen | Estrogen | Many breast cancers have | Breast cancer |
| (fulvestrant) | receptor | receptor | estrogen receptors (ER), and | |
| | antagonist | | the growth of these tumors | |
| | | | can be stimulated by | |
| | | | estrogen. Fulvestrant is an | |
| | | | estrogen receptor antagonist | |
| | | | that binds to the estrogen | |
| | | | receptor in a competitive | |
| | | | manner with affinity | |
| | | | comparable to that of | |
| | | | estradiol. Fulvestrant down- | |
| | | | regulates the ER protein in | |
| | | | human breast cancer cells. | |
| Gemzar ® | nucleoside | Ribonucleotide | Exhibits cell phase | Non-small cell |
| (gemcitabine | analogue | reductase | specificity, primarily killing | lung cancer; |
| HCl) | that exhibits | | cells undergoing DNA | adenocarcinoma |
| | antitumor | | synthesis (S-phase) and also | of the pancreas |
| | activity | | blocking the progression of | |
| | | | cells through the G1/S-phase | |
| | | | boundary. Gemcitabine is | |
| | | | metabolized intracellularly | |
| | | | by nucleoside kinases to the | |
| | | | active diphosphate | |
| | | | (dFdCDP) and triphosphate | |
| | | | (dFdCTP) nucleosides. The | |
| | | | cytotoxic effect of | |
| | | | gemcitabine is attributed to a | |
| | | | combination of two actions | |
| | | | of the diphosphate and the | |
| | | | triphosphate nucleosides, | |
| | | | which leads to inhibition of | |
| | | | DNA synthesis. First, | |
| | | | gemcitabine diphosphate | |
| | | | inhibits ribonucleotide | |
| | | | reductase, which is | |
| | | | responsible for catalyzing the | |
| | | | reactions that generate the | |
| | | | deoxynucleoside | |
| | | | triphosphates for DNA | |
| | | | synthesis. Inhibition of this | |
| | | | enzyme by the diphosphate | |
| | | | nucleoside causes a | |
| | | | reduction in the | |
| | | | concentrations of | |
| | | | deoxynucleotides, including | |
| | | | dCTP. Gemcitabine | |
| | | | triphosphate also competes | |
| | | | with dCTP for incorporation | |
| | | | into DNA. The reduction in | |
| | | | the intracellular | |
| | | | concentration of dCTP | |
| | | | enhances the incorporation | |
| | | | of gemcitabine triphosphate | |
| | | | into DNA. After the | |
| | | | gemcitabine nucleotide is | |
| | | | incorporated into DNA, only | |
| | | | one additional nucleotide is | |
| | | | added to the growing DNA | |
| | | | strands, after which there is | |
| | | | inhibition of DNA synthesis. | |
| Purinethol | Inhibits | Hypoxanthine- | Mercaptopurine competes | acute lymphatic |
| (mercaptopurine) | purine | guanine | with hypoxanthine and | leukemia |
| | synthesis | phosphoribosyltransferase | guanine for the enzyme | |
| | | (HGPRTase) | hypoxanthine-guanine | |
| | | | phosphoribosyltransferase | |
| | | | (HGPRTase). See next entry | |
| Thioguanine | Inhibits | Hypoxanthine- | Competes with hypoxanthine | acute |
| | purine | guanine | and guanine for the enzyme | nonlymphocytic |
| | synthesis | phosphoribosyltransferase | hypoxanthine-guanine | leukemias |
| | | (HGPRTase) | phosphoribosyltransferase | |
| | | | (HGPRTase) and is | |
| | | | converted to 6-thioguanylic | |
| | | | acid (TGMP). TGMP | |
| | | | interferes at several points | |
| | | | with the synthesis of guanine | |
| | | | nucleotides. It inhibits de | |
| | | | novo purine biosynthesis by | |
| | | | pseudo-feedback inhibition | |
| | | | of glutamine-5- | |
| | | | phosphoribosyl- | |
| | | | pyrophosphate | |
| | | | amidotransferase resulting in | |
| | | | a sequential blockade of the | |
| | | | synthesis and utilization of | |
| | | | the purine nucleotides. | |

### (b) In Vivo Screening

Following identification in plate-based in vitro assays, selected siRNAs may be further tested in art-accepted animal model systems to assess gene silencing effects in vivo. Exemplary animal model systems include xenomouse models wherein specific human tumors (non drug resistant and drug resistant) are propagated in select mouse strains. The testing involves the delivery of the siRNA to its specific target and measuring the silencing effect of the gene. Measuring of the silencing effect may, for example, be achieved by flow cytometry and/or plate based assays.

SEQ ID NO: 1-5308 are set out in the attached Sequence Listing. The codes for polynucleotide and polypeptide sequences used in the attached Sequence Listing confirm to WIPO Standard ST.25 (1988), Appendix 2.

All references cited herein, including patent references and non-patent references, are hereby incorporated by reference in their entireties.

1. A composition comprising a small interfering nucleic acid molecule (siNA) that is capable of reducing expression of a target gene wherein the target gene encodes a protein selected from the group consisting of: hexokinase 2 (HK-II); lactate dehydrogenase 5 (LDHA); glucose-6-phosphate dehydrogenase (H6PD); phosphofructokinase 3 (PFKL); 5-phosphoribosyl-1-pyrophosphate synthetase (PRPS); amido-phosphoribosyltransferase (PPAT); carbamoyl phosphate synthetase 1 (CPS1); carbamoyl phosphate synthetase 2 (CAD); ribonucleotide reductase subunit 1 (RRM1); ribonucleotide reductase subunit 2 (RRM2); ribonucleotide reductase subunit 2B (RRM2B); thymidylate synthetase (TYMS); dihydrofolate reductase (DHFR); adenylate kinase (AK1); NAD synthase (NADSYN); flavin adenine dinucleotide (FAD) synthetase (FLAD1); NADH-Q reductase; cytochrome reductase (UQCRC2); cytochrome oxidase (COX5A); ATP synthase (F0F1-ATPase); beta chain (ATP5B); fatty acid synthase (FASN); ATP citrate lyase (ACLY); ATP-ADP translocase 1 (ANT1); ATP-ADP translocase 2 (ANT2); ATP-ADP translocase 3 (ANT3); CTP synthase (CTPS); inosine monophosphate dehydrogenase 2 (IMPD2); deoxycytidine kinase (DCK); thymidine kinase 1 (TK1); thymidine kinase 2 (TK2); deoxyguanosine kinase (DGUOK); uridine monophosphate synthase (UMPS); dihydropyrimidine dehydrogenase (DPYD); HIF1α (HIF1A); ARNT (HIF1B); GLUT1 (SLC2A1); GLUT3 (SLC2A3); GLUT5 (SLC2A5); GLUT12 (SLC2A12); ENT1 (SLC29A1); ENT2 (SLC29A2); ENT3 (SLC29A3); ENT4 (SLC29A4); CNT1 (SLC28A1); CNT2 (SLC28A2); CNT3 (SLC28A3); ASCT1 (SLC1A4); ASCT2 (SLC1A5); 4F2hc (SLC3A2); ATB^{0,+} (SLC6A14); LAT1 (SLC7A5); xCT (SLC7A11); LAT3 (SLC43A1); SNAT3 (SLC43A4); SNAT5 (SLC43A5); SMVT1 (SLC5A6); RFC(SLC19A1); ThTr1 (SLC19A1); ThTr2 (SLC19A2); SVCT1 (SLC23A1); SVCT2 (SLC23A2); CIC (SLC25A1); DIC (SLC25A10); NHE1 (SLC9A1); MCT1 (SLC16A1); PMCA1 (ATP2B1); PMCA2 (ATP2B2); PMCA4 (ATP2B4); V-type proton pump (subunit proteins encoded by ATP6V1A, ATP6V1B2, ATP6V1C1, ATP6V1E1, ATP6V1F); ATP7A; ATP7B; MDR1 (ABCB1); MRP4 (ABCC4); MRP5 (ABCC5); and YBX1.

2. The composition of claim 1, wherein the target gene is selected from the group consisting of SEQ ID NO: 1 to 15 and 17 to 92.

3. The composition of claim 1, wherein the siNA is targeted against a region of the target gene selected from the group consisting of: 5′ untranslated regions; coding regions; 3′ untranslated regions; and promoter regions.

4. The composition of claim 1, wherein the siNA is directed against a target sequence selected from the group consisting of: SEQ ID NO: 1 to 15 and 17 to 92.

5. (canceled)

6. The composition of claim 1, wherein the siNA is from 19 to 30 nucleotides in length.

7. The composition of claim 6, wherein the siNA is from 19 to 25 nucleotides in length.

8. The composition of claim 6, wherein the siNA is from 26 to 30 nucleotides in length.

9. The composition of claim 6, wherein the siNA is 27 nucleotides in length.

10. The composition of claim 1, wherein the siNA is chemically modified.

11. The composition of claim 1, wherein the siNA is a double-stranded RNA molecule.

12. The composition of claim 1, further comprising a nucleic acid binding component that condenses and protects the siNA.

13. The composition of claim 1, wherein the siNA is formulated as a liposome.

14. The composition of claim 13, wherein the liposome is a pegylated liposome.

15. The composition of claim 1, wherein the siNA is attached to a lipid or polymer carrier.

16. The composition claim 1, further comprising a binding agent that specifically binds to a target internalizable antigen expressed on the surface of a target cell of interest.

17. The composition of claim 1, further comprising a physiologically acceptable carrier.

18. The composition of claim 1, further comprising a chemotherapeutic agent.

19. A composition comprising a genetic construct that is capable of expressing a short-hairpin RNA (shRNA) which is processed within the cell to a small interfering nucleic acid molecule (siNA), wherein the siNA is capable of reducing expression of a target gene that encodes a protein selected from the group consisting of: hexokinase 2 (HK-II); lactate dehydrogenase 5 (LDHA); glucose-6-phosphate dehydrogenase (H6PD); phosphofructokinase 3 (PFKL); 5-phosphoribosyl-1-pyrophosphate synthetase (PRPS); amido-phosphoribosyltransferase (PPAT); carbamoyl phosphate synthetase 1 (CPS1); carbamoyl phosphate synthetase 2 (CAD); ribonucleotide reductase subunit 1 (RRM1); ribonucleotide reductase subunit 2 (RRM2); ribonucleotide reductase subunit 2B (RRM2B); thymidylate synthetase (TYMS); dihydrofolate reductase (DHFR); adenylate kinase (AK1); NAD synthase (NADSYN); flavin adenine dinucleotide (FAD) synthetase (FLAD1); NADH-Q reductase; cytochrome reductase (UQCRC2); cytochrome oxidase (COX5A); ATP synthase (F0F1-ATPase); beta chain (ATP5B); fatty acid synthase (FASN); ATP citrate lyase (ACLY); ATP-ADP translocase 1 (ANT1); ATP-ADP translocase 2 (ANT2); ATP-ADP translocase 3 (ANT3); CTP synthase (CTPS); inosine monophosphate dehydrogenase 2 (IMPD2); deoxycytidine kinase (DCK); thymidine kinase 1 (TK1); thymidine kinase 2 (TK 2); deoxyguanosine kinase (DGUOK); uridine monophosphate synthase (UMPS); dihydropyrimidine dehydrogenase (DPYD); HIF1α: (HIF1A); ARNT (HIF1B); GLUT1 (SLC2A1); GLUT3 (SLC2A3); GLUT5 (SLC2A5); GLUT12 (SLC2A12); ENT1 (SLC29A1); ENT2 (SLC29A2); ENT3 (SLC29A3); ENT4 (SLC29A4); CNT1 (SLC28A1); CNT2 (SLC28A2); CNT3 (SLC28A3); ASCT1 (SLC1A4); ASCT2 (SLC1A5); 4F2hc (SLC3A2); ATB^{0,+} (SLC6A14); LAT1 (SLC7A5); xCT (SLC7A11); LAT3 (SLC43A1); SNAT3 (SLC43A4); SNAT5 (SLC43A5); SMVT1 (SLC5A6); RFC (SLC19A1); ThTr1 (SLC19A1); ThTr2 (SLC19A2); SVCT1 (SLC23A1); SVCT2 (SLC23A2); CIC (SLC25A1); DIC (SLC25A10); NHE1 (SLC9A1); MCT1 (SLC16A1); PMCA1 (ATP2B1); PMCA2 (ATP2B2); PMCA4 (ATP2B4); V-type proton pump (subunit proteins encoded by ATP6V1A, ATP6V1B2, ATP6V1C1, ATP6V1E1, ATP6V1F); ATP7A; ATP7B; MDR1 (ABCB1); MRP4 (ABCC4); MRP5 (ABCC5); and YBX1.

20. The composition of claim 19, wherein the genetic construct comprises a promoter that is specific to a target cell of interest.

21. The composition of claim 19, wherein the target gene is selected from the group consisting of: SEQ ID NO: 1 to 15 and 17 to 92.

22. The composition of claim 19, wherein the siNA is targeted against a region of the target gene selected from the group consisting of: 5′ untranslated regions; coding regions; 3′ untranslated regions; and promoter regions.

23. The composition of claim 19, wherein the siNA is directed against a target sequence selected from the group consisting of: SEQ ID NO: 1 to 15 and 17 to 92.

24. (canceled)

25. The composition of claim 19, wherein the siNA is from 19 to 30 nucleotides in length.

26. The composition of claim 25, wherein the siNA is from 19 to 25 nucleotides in length.

27. The composition of claim 25, wherein the siNA is from 26 to 30 nucleotides in length.

28. The composition of claim 25, wherein the siNA is 27 nucleotides in length.

29. The composition of claim 19, further comprising a binding agent that specifically binds to a target internalizable antigen expressed on the surface of target cell of interest.

30. The composition of claim 17, further comprising a physiologically acceptable carrier.

31. The composition of claim 17, further comprising a chemotherapeutic agent.

32. A method for the treatment of a disorder in a patient, comprising administering to the patient a composition of claim 1.

33. The method of claim 32, further comprising administering a chemotherapeutic agent.

34. The method of claim 32, wherein the disorder is a cancer.

35. The method of claim 34, wherein the cancer selected from the group consisting of: primary and metastatic tumors and carcinomas of the breast, colon, rectum, lung, oropharynx, hypopharynx, esophagus, stomach, pancreas, liver, gall bladder, bile ducts, small intestine, kidney, bladder, urothelium, cervix, uterus, ovaries, prostate, seminal vesicles, testes, endocrine glands, head, neck and skin; tumors of the brain, nerves, eyes or meninges; astrocytomas; gliomas; glioblastomas; retinoblastomas; neuromas; neuroblastomas; solid tumors arising from hematopoietic malignancies; leukemia; myeloma; melanoma and lymphomas.

36. A method for the treatment of a disorder in a patient, comprising administering to the patient a composition of claim 19.

37. The method of claim 36, further comprising administering a chemotherapeutic agent.

38. The method of claim 36, wherein the disorder is a cancer.

39. The method of claim 38, wherein the cancer selected from the group consisting of: primary and metastatic tumors and carcinomas of the breast, colon, rectum, lung, oropharynx, hypopharynx, esophagus, stomach, pancreas, liver, gall bladder, bile ducts, small intestine, kidney, bladder, urothelium, cervix, uterus, ovaries, prostate, seminal vesicles, testes, endocrine glands, head, neck and skin; tumors of the brain, nerves, eyes or meninges; astrocytomas; gliomas; glioblastomas; retinoblastomas; neuromas; neuroblastomas; solid tumors arising from hematopoietic malignancies; leukemia; myeloma; melanoma and lymphomas.

40. A method for identifying an siNA molecule for the treatment of a disease, the method comprising:
(a) generating a population of candidate siNA molecules directed against a target gene; and
(b) comparing the in vitro efficacy candidate siNA molecules, alone or in combination, with the in vitro efficacy of the chemotherapeutic drug; and
(c) comparing the in vitro efficacy of the siNA(s) alone or the chemotherapeutic drug alone with that of a treatment comprising a combination of siNA(s) with the chemotherapeutic drug.

41. The method of claim 40, further comprising comparing the in vivo efficacy of at least one of the candidate siNA molecules with the in vivo efficacy of the chemotherapeutic drug in an animal model system for the disease, either as single agents (siNA alone or the chemotherapeutic drug alone) or as combinations (multiple siNAs or siNA with the chemotherapeutic drug).

42. The method of claim 40, wherein the target gene is selected from the group consisting of:
(a) genes that are part of a biochemical or metabolic process;
(b) genes that are involved in resistance to chemotherapeutic drugs; and
(c) genes that encode a protein product that is a target of a chemotherapeutic drug that is known to be effective in treatment of the disease.

43. The method of claim 42, wherein the protein product is selected from the group consisting of: hexokinase 2 (HK-II); lactate dehydrogenase 5 (LDHA); glucose-6-phosphate dehydrogenase (H6PD); phosphofructokinase 3 (PFKL); 5-phosphoribosyl-1-pyrophosphate synthetase (PRPS); amido-phosphoribosyltransferase (PPAT); carbamoyl phosphate synthetase 1 (CPS1); carbamoyl phosphate synthetase 2 (CAD); ribonucleotide reductase subunit 1 (RRM1); ribonucleotide reductase subunit 2 (RRM2); ribonucleotide reductase subunit 2B (RRM2B); thymidylate synthetase (TYMS); dihydrofolate reductase (DHFR); adenylate kinase (AK1); NAD synthase (NADSYN); flavin adenine dinucleotide (FAD) synthetase (FLAD1); NADH-Q reductase; cytochrome reductase (UQCRC2); cytochrome oxidase (COX5A); ATP synthase (F0F1-ATPase); beta chain (ATP5B); fatty acid synthase (FASN); ATP citrate lyase (ACLY); ATP-ADP translocase 1 (ANT1); ATP-ADP translocase 2 (ANT2); ATP-ADP translocase 3 (ANT3); CTP synthase (CTPS); inosine monophosphate dehydrogenase 2 (IMPD2); deoxycytidine kinase (DCK); thymidine kinase 1 (TK1); thymidine kinase 2 (TK 2); deoxyguanosine kinase (DGUOK); uridine monophosphate synthase (UMPS); dihydropyrimidine dehydrogenase (DPYD); HIF1α (HIF1A); ARNT (HIF1B); GLUT1 (SLC2A1); GLUT3 (SLC2A3); GLUT5 (SLC2A5); GLUT12 (SLC2A12); ENT1 (SLC29A1); ENT2 (SLC29A2); ENT3 (SLC29A3); ENT4 (SLC29A4); CNT1 (SLC28A1); CNT2 (SLC28A2); CNT3 (SLC28A3); ASCT1 (SLC1A4); ASCT2 (SLC1A5); 4F2hc (SLC3A2); ATB^{0,+} (SLC6A14); LAT1 (SLC7A5); xCT (SLC7A11); LAT3 (SLC43A1); SNAT3 (SLC43A4); SNAT5 (SLC43A5); SMVT1 (SLC5A6); RFC (SLC19A1); ThTr1 (SLC19A1); ThTr2 (SLC19A2); SVCT1 (SLC23A1); SVCT2 (SLC23A2); CIC (SLC25A1); DIC (SLC25A10); NHE1 (SLC9A1); MCT1 (SLC16A1); PMCA1 (ATP2B1); PMCA2 (ATP2B2); PMCA4 (ATP2B4); V-type proton pump (subunit proteins encoded by ATP6V1A, ATP6V1B2, ATP6V1C1, ATP6V1E1, ATP6V1F); ATP7A; ATP7B; MDR1 (ABCB1); MRP4 (ABCC4); MRP5 (ABCC5); and YBX1.

44. The method of claim 40, wherein the chemotherapeutic drug is selected from the group consisting of: capecitabine, methotrexate, fluorouridine, 5-fluorouracil, gemcitabine, doxorubicin, etoposide and cisplatin.
